(19) United States
(12) Patent Application Publication
(10) Pub. No.: US 2026/0287577 A1
(43) Pub. Date: Sep. 24, 2026

(54) APPLICATIONS OF HEPATOCYTE CO-CULTURE SYSTEM

(71) Applicant: LifeNet Health, Virginia Beach, VA (US)

(72) Inventors: Jung Bok LEE, Virginia Beach, VA (US); Jingsong CHEN, Virginia Beach, VA (US); Edward LECLUYSE, Virginia Beach, VA (US); Angela MURCHISON, Virginia Beach, VA (US); Jessica WEAVER, Virginia Beach, VA (US); Justin ODANGA, Virginia Beach, VA (US); Kristina WOLF, Virginia Beach, VA (US); Sharon PRESNELL, Virginia Beach, VA (US); James MCKIM, Virginia Beach, VA (US)

(21) Appl. No.: 19/168,263

(22) PCT Filed: Mar. 22, 2024
(86) PCT No.: PCT/US2024/021192

§ 371 Date: Sep. 23, 2025

Related U.S. Application Data
(60) Provisional application No. 63/454,529, filed on Mar. 24, 2023.
(60) Provisional application No. 63/597,923, filed on Nov. 10, 2023.
(60) Provisional application No. 63/560,739, filed on Mar. 3, 2024.
(60) Provisional application No. 63/562,974, filed on Mar. 8, 2024.

(51) Int. Cl. G01N 33/50; C12N 5/071

(52) U.S. Cl. CPC G01N 33/5067; C12N 5/067; G01N 33/5014; G01N 33/5038; C12N 2502/1394; C12N 2502/28; C12N 2503/02; G01N 2500/10

(57) ABSTRACT

The present invention provides a product comprising plated human hepatocytes on a surface and at least some of the plated hepatocytes are in one or more hepatocyte clusters on feeder cells, which are attached to the surface. The plated human hepatocytes may be neonatal, juvenile, or adult hepatocytes. The product may include macrophage, e.g., liver-derived macrophage (e.g., Kupffer cells), or other hepatic non-parenchymal cells (NPCs) (e.g., stellate cells or liver endothelial cells). A method of preparing the plated human hepatocytes is provided. The method includes applying human hepatocytes to a surface in the presence of feeder cells, co-culturing the applied hepatocytes with the feeder cells, and forming one or more hepatocyte clusters by the co-cultured hepatocytes on the feeder cells, which are attached to the surface. The plated hepatocytes may be used for various purposes, such as preparation of a hepatitis B virus (HBV) infected hepatocyte culture model and drug testing.

### CROSS-REFERENCE TO RELATED APPLICATIONS

This application is a United States National Stage Application of International PCT Application No. PCT/US2024/021192 titled “APPLICATIONS OF HEPATOCYTE CO-CULTURE SYSTEM” filed Mar. 22, 2024, which claims priority to: U.S. Provisional Patent Application No. 63/562,974, filed Mar. 8, 2024, titled “APPLICATIONS OF HEPATOCYTE CO-CULTURE SYSTEM;” U.S. Provisional Patent Application No. 63/560,739, filed Mar. 3, 2024, titled “APPLICATIONS OF HEPATOCYTE CO-CULTURE SYSTEM;” U.S. Provisional Patent Application No. 63/597,923, filed Nov. 10, 2023, titled “APPLICATIONS OF HEPATOCYTE CO-CULTURE SYSTEM;” and U.S. Provisional Patent Application No. 63/454,529, filed Mar. 24, 2023, titled “APPLICATIONS OF HEPATOCYTE CO-CULTURE SYSTEM;” the entire contents of each of which are incorporated herein by reference in their entireties for all purposes.

### FIELD OF THE INVENTION

The invention relates generally to plated human hepatocytes and preparation and uses thereof.

### BACKGROUND OF THE INVENTION

Primary human hepatocytes isolated from donors have been used in 2D in vitro culture systems for studying drug metabolism or drug induced liver injury. For example, liver fibrosis may be a result of chronic hepatic injury due to various conditions (e.g., viral infection, metabolic disease), as well as drug and chemical exposure. Mechanisms underlying this disease are complex and involve multiple cell types. In vivo animal models have been the gold standard for studying liver fibrosis, but do not fully mimic the immunogenic and metabolic complexities in humans. Most advanced or multicellular in vitro culture systems, including the organ-on-a-chip system, other co-culture platforms, and liver organoids, lack the critical cellular composition to elicit these required response mechanisms or do not allow for high-throughput screening of potential compound-induced fibrogenic responses in a cost-effective or physiologically relevant manner. In 2D in vitro culture systems, primary human hepatocytes are maintained as mono-cultures (single cell type) in a culture medium and attach to a surface (e.g., a plate) to generate a monolayer of the hepatocytes suitable for testing drug metabolism, toxicity or viral infection. For 2D in vitro culture systems, isolated primary human hepatocytes having a plateability of at least ≥85% for a prolonged period of time, for example, at least 1, 2, 3, 4, 5, 6, 7, 14, 21, 28, 35, 42 or 49 days, are desirable while isolated primary hepatocytes having a plateability of less than 60% are generally deemed less desirable and not suited for long-term experiments. A high percentage of cryopreserved primary human hepatocyte batches fall into the latter category of non- or poorly plateable hepatocytes that limits the availability of many batches with desirable donor specifications such as Metabolic dysfunction-Associated Fatty Liver Disease (MAFLD) and metabolic dysfunction-associated steatohepatitis (MASH) for the applications such as testing drug metabolism, toxicity or viral infection. There is a need to improve the plateability, functional stability and culture longevity of primary human hepatocytes.

Liver diseases, including MAFLD, are a growing worldwide health concern. Currently, there is a lack of suitable in vitro models that sustain basic primary human hepatocyte (PHH) morphology and functionality while supporting presentation of disease-associated phenotypic characteristics such as lipid accumulation and inflammasome activation. In TruVivo®, an all-human triculture system (hTCS), basic metabolic functions were characterized in PHHs isolated from normal or diseased livers during two-weeks of culture. Decreases in albumin and urea levels and CYP3A4 activity were seen in diseased-origin PHHs compared to normal PHHs along with higher CYP2E1 expression. Positive expression of the macrophage markers CD68 and CD163 were seen in the diseased PHH preparations. Elevated levels of the pro-inflammatory cytokines IL-6 and MCP-1 and the fibrotic markers CK-18 and TGF-β were also measured. Gene expression of FASN, PCK1, and G6PC in the diseased PHHs was decreased compared to the normal PHHs. Further characterization revealed differences in lipogenesis and accumulation of intracellular lipids in normal and diseased PHHs when cultured with oleic acid and high glucose. TruVivo® represents a promising new platform to study lipogenic mechanisms in normal and diseased populations due to the preservation of phenotypic differences over a prolonged culture period.

Obesity and chronic liver injury are major global health concerns with increasing prevalence. Metabolic dysfunction-Associated Fatty Liver Disease (MAFLD) represents a substantial portion of the healthcare burden in Western countries afflicting 20 to 30% of the general population. The spectrum of MAFLD is wide ranging and includes benign simple steatosis to metabolic dysfunction-associated steatohepatitis (MASH), fibrosis, cirrhosis, and hepatocellular carcinoma (HCC). The increasing number of toxic chemicals and current upward trend of obesity and metabolic syndrome make liver disease difficult to study due to the lack of suitable in vitro models. Animal disease models have been used to fill this gap; however, the interspecies differences do not accurately model human disease due to the use of artificial induction regimes and/or the lack of consistent human-relevant pathogenesis. Inducing disease in mouse models with a high-fat diet can have varied outcomes depending on the composition of the diet and feeding length, and may not result in inflammation and fibrosis. Additionally, disease development for MASH and HCC has been inconsistent due to the dependence on the genetic background of the mouse strain and the unreliable development of progressive chronic disease. With the limitations of existing animal models, there is a need for improving human liver model systems that better mimic key elements of human MAFLD.

One complication of in vitro models is the lack of an accurate and consistent diseased phenotype. Some 3D model systems, including spheroids and liver-on-a-chip, are highly complex, difficult to reproduce, and costly. A model system is needed that maintains diseased morphology and characteristics including lipid accumulation and inflammation for extended culture periods. In the early stages of MAFLD, accumulation of lipids in hepatocytes by excess dietary fats, absorption of free fatty acids (FFAs), lipogenesis, or insufficient fatty acid oxidation can result in steatosis and insulin resistance. Once steatosis begins, reactive oxygen species (ROS) are generated, leading to activation of immune cells resulting in inflammation and liver injury, the second phase of MAFLD. These immune cells, including macrophages, secrete pro-inflammatory cytokines leading to increased levels of collagen I, α-smooth muscle actin and transforming growth factor-β (TGF-β). The resulting deposition of extracellular matrix and fibrosis is the third stage of MAFLD. A physiologically-relevant diseased model system should progress to display features from all 3 stages of MAFLD.

### SUMMARY OF THE INVENTION

The present invention relates to plated human hepatocytes and preparation and uses thereof. The hepatocytes may be co-cultured with feeder cells derived from human stromal and endothelial tissue sources.

Recent development of TruVivo®, an all-human cell-based triculture system (hTCS) that includes cryopreserved primary human feeder cells (FCs) and primary human hepatocytes (PHHs) may be used to characterize a normal liver system. According to one embodiment, in this hTCS, PHHs from healthy donor tissues maintained histotypic morphology for greater than 42 days with no Matrigel overlay. They showed stable basic hepatocyte function, including albumin and urea production, Cytochrome P450 (CYP) 3A4 activity, and formed anastomosing networks of bile canaliculi with tight and gap junctions. Overall, the hTCS maintained the original phenotype of the specific donor lots of PHHs over prolonged periods of culture time.

A first product comprising plated hepatocytes on a surface is provided. The plated hepatocytes are adult, neonatal or juvenile hepatocytes. At least 70% of the plated hepatocytes are in one or more hepatocyte clusters on feeder cells. The feeder cells are endothelial cells and stromal cells (e.g., fibroblasts) from human tissue sources, and are attached to the surface. The plated hepatocytes and the feeder cells are obtained from one or more donors.

A second product comprising (a) plated hepatocytes on a surface and (b) macrophages, for example, liver-derived macrophage (e.g., Kupffer cells), or other hepatic non-parenchymal cells (NPCs) (e.g., stellate cells or liver endothelial cells) is provided. At least 70% of the plated hepatocytes are in one or more hepatocyte clusters on feeder cells. The feeder cells are endothelial cells and stromal cells (e.g., fibroblasts), and are attached to the surface. The plated hepatocytes and the feeder cells are obtained from one or more donors. The ratio of the macrophages, for example, liver-derived macrophage (e.g., Kupffer cells), or other hepatic non-parenchymal cells (NPCs) (e.g., stellate cells or liver endothelial cells) to the plated hepatocytes may be between about 1:100 and about 1:2, for example, 1:100, 1:80, 1:50, 1:20, 1:10 or 1:2. The ratio of the Kupffer cells or other liver-derived macrophages to the plated hepatocytes may be between about 1:100 and about 4:1, or from about 1:100 to about 2:1 or from about 1:100 to about 1:1, or from about 1:100 to about 1:2.

The macrophages, for example, liver-derived macrophage (e.g., Kupffer cells), or other hepatic non-parenchymal cells (NPCs) (e.g., stellate cells or liver endothelial cells) may be from a donor having a healthy liver or a liver with a disease or a condition selected from the group consisting of microsteatosis, metabolic dysfunction-associated steatohepatitis (MASH), Metabolic dysfunction-Associated Fatty Liver Disease (MAFLD)) and hepatitis. The macrophages, for example, liver-derived macrophage (e.g., Kupffer cells), or other hepatic non-parenchymal cells (NPCs) (e.g., stellate cells or liver endothelial cells) may be from a healthy donor, or a donor who shows a pathology selected from the group consisting of metabolic dysfunction-associated steatohepatitis (MASH), Metabolic dysfunction-Associated Fatty Liver Disease (MAFLD)), alcoholic steatohepatitis (ASH), alpha-1-antitrypsin deficiency, Wilson's disease, Hepatitis B, Hepatitis C, Hemochromatosis, hyperoxaluria, autoimmune disease, cancer, obesity, type 2 diabetes, or an inborn error of metabolism. The Kupffer cells or other liver-derived macrophages may be from a healthy donor. The Kupffer cells or other liver-derived macrophages may be from a donor having a liver with a disease or a condition.

According to the first or second product, the plated hepatocytes may be neonatal hepatocytes. The plated hepatocytes may be juvenile hepatocytes. The plated hepatocytes may be adult hepatocytes. The plated hepatocytes may be obtained from a single donor. The plated hepatocytes may be obtained from two or more donors.

A method of testing a pharmaceutical substance is provided. The method comprises administering a pharmaceutical substance to the plated hepatocytes in the first or second product of the present invention in an amount effective to change a property of the plated hepatocytes or the adjoining feeder cells or both. The pharmaceutical substance may be selected from the group consisting of small molecules, lipid nanoparticles, antibodies, live viruses, viral vectors, bacteria or derivatives thereof, oligonucleotides and cells. A purpose of this study was to develop an in vitro strategy using a novel all-human 2D+ hepatic system, comprised of a human source of hepatocytes and feeder cells with Kupffer cells (KCs) to assess initial fibrogenic responses following compound treatment.

A method of testing drug metabolism is provided. The method comprises administering an effective amount of a drug to the plated hepatocytes in the first or second product of the present invention in a culture medium, and determining the amount of the drug or drug metabolites in the plated hepatocytes or culture medium.

A method of testing drug transport is provided. The method comprises administering an effective amount of a drug to the plated hepatocytes in the first or second product of the present invention, and determining the location of the drug in the plated hepatocytes.

A method of testing drug toxicity is provided. The method comprises administering an effective amount of a drug to the plated hepatocytes in the first or second product of the present invention, and detecting viable plated hepatocytes, viable feeder cells, or other cytotoxicity biomarkers.

A method of testing a pathogenic agent is provided. The method comprises administering an effective amount of an agent to the plated hepatocytes in the first or second product of the present invention, and detecting a disease biomarker.

A first kit for plating hepatocytes is provided. The first kit comprises (a) a first cryovial comprising primary hepatocytes, which are adult, neonatal or juvenile hepatocytes; (b) a second cryovial comprising endothelial cells; (c) a third cryovial comprising stromal cells (e.g., fibroblasts); and (d) an instruction for preparing plated hepatocytes with the primary hepatocytes, the endothelial cells and the stromal cells (e.g., fibroblasts) according to a method comprising: (i) applying the primary hepatocytes to a surface in the presence of feeder cells, wherein the feeder cells are the endothelial cells and the stromal cells (e.g., fibroblasts), (ii) co-culturing the applied hepatocytes with the feeder cells after step (i), and (iii) forming one or more hepatocyte clusters by the co-cultured hepatocytes on the feeder cells such that the feeder cells are attached to the surface, and at least 85% of the co-cultured hepatocytes are in the one or more hepatocyte clusters. According to another embodiment, the feeder cells, i.e., the endothelial cells and the stromal cells (e.g. fibroblasts) may be provided together in a single vial.

A second kit for plating hepatocytes is provided. The second kit comprises (a) a first cryovial comprising primary hepatocytes; (b) a second cryovial comprising endothelial cells; (c) a third cryovial comprising stromal cells (e.g., fibroblasts); (d) a fourth cryovial comprising macrophages, for example, liver-derived macrophage (e.g., Kupffer cells), or other hepatic non-parenchymal cells (NPCs) (e.g., stellate cells or liver endothelial cells); and (e) an instruction for preparing plated hepatocytes with the primary hepatocytes, the endothelial cells and the stromal cells (e.g., fibroblasts) according to a method comprising: (i) applying the primary hepatocytes to a surface in the presence of feeder cells, wherein the feeder cells are the endothelial cells and the stromal cells (e.g., fibroblasts), (ii) co-culturing the applied hepatocytes with the feeder cells after step (i), and (iii) forming one or more hepatocyte clusters by the co-cultured hepatocytes on the feeder cells such that the feeder cells are attached to the surface, and at least 85% of the co-cultured hepatocytes are in the one or more hepatocyte clusters. According to another embodiment, the feeder cells, i.e., the endothelial cells and the stromal cells (e.g. fibroblasts) may be provided together in a single vial. The ratio of the macrophages, for example, liver-derived macrophage (e.g., Kupffer cells), or other hepatic non-parenchymal cells (NPCs) (e.g., stellate cells or liver endothelial cells) to the plated hepatocytes may be between about 1:100 and about 1:2, for example, 1:100, 1:80, 1:50, 1:20, 1:10, or 1:2. The ratio of the Kupffer cells or other liver-derived macrophages to the plated hepatocytes may be between about 1:100 and about 4:1, or from about 1:100 to about 2:1 or from about 1:100 to about 1:1, or from about 1:100 to about 1:2. The macrophages, for example, liver-derived macrophage (e.g., Kupffer cells), or other hepatic non-parenchymal cells (NPCs) (e.g., stellate cells or liver endothelial cells) may be from a donor having a healthy liver or a liver with a disease or a condition selected from the group consisting of microsteatosis, metabolic dysfunction-associated steatohepatitis (MASH), Metabolic dysfunction-Associated Fatty Liver Disease (MAFLD) and hepatitis. The macrophages, for example, liver-derived macrophage (e.g., Kupffer cells), or other hepatic non-parenchymal cells (NPCs) (e.g., stellate cells or liver endothelial cells) may be from a healthy donor or a donor who shows a pathology selected from the group consisting of metabolic dysfunction-associated steatohepatitis (MASH), Metabolic dysfunction-Associated Fatty Liver Disease (MAFLD), alcoholic steatohepatitis (ASH), alpha-1-antitrypsin deficiency, Wilson's disease, Hepatitis B, Hepatitis C, Hemochromatosis, hyperoxaluria, autoimmune disease, cancer, obesity, type 2 diabetes, or other inborn errors of metabolism.

According to the first or second kit, the hepatocytes may be neonatal hepatocytes. The hepatocytes may be juvenile hepatocytes. The hepatocytes may be adult hepatocytes. The second cryovial and the third cryovial may be the same.

### BRIEF DESCRIPTION OF THE DRAWINGS

FIG. 1 shows schematic #1 for a human hepatocyte co-culture according to one embodiment of the invention. This schematic provides the preparation of the cell culture and analysis with a timeframe. According to schematic #1, the hepatocyte co-culture is prepared by seeding human hepatocytes onto pre-plated feeder cells, which are endothelial cells and fibroblasts, on day 0. ECs, endothelial cells; Fbs, fibroblasts; MG, MATRIGEL.

FIG. 2 shows schematic #2 for a human hepatocyte co-culture according to another embodiment of the invention. This schematic provides the preparation of the cell culture and analysis with a timeframe. According to schematic #2, the hepatocyte co-culture is prepared by seeding human hepatocytes together with feeder cells, which are endothelial cells and fibroblasts, on day 0. ECs, endothelial cells; Fbs, fibroblasts; MG, MATRIGEL.

FIG. 3 shows morphology of human cells used for a hepatocyte co-culture: dermal fibroblasts alone (top left), immortalized liver sinusoidal endothelial cells alone (top center), primary hepatocytes alone (top right), a mixture of fibroblasts and endothelial cells (bottom left), and a mixture of primary hepatocytes, endothelial cells and fibroblasts (bottom right).

FIG. 4 shows expression levels of albumin, CYP450 1A2, CYP450 2B6, and CYP450 3A4 genes in a hepatocyte mono-culture (mono-culture), a hepatocyte co-culture with feeder cells (co-culture), or a feeder cell culture (feeder) a week after plating as measured by qRT-PCR. The expression level of each of these four genes was significantly higher in the co-culture than the mono-culture. *, p<0.05; **, p<0.01.

FIG. 5 shows normalized CYP450 1A2, CYP450 2B6, or CYP450 3A4 activities based on seeded hepatocyte numbers in mono-culture or co-culture seven days after plating before and after induction. *, p<0.05; **, p<0.01.

FIG. 6 shows secretion of albumin (ALB) and urea measured over 2 weeks of hepatocyte culture. The levels of secreted albumin and urea were significantly higher from hepatocytes in co-culture compared to mono-culture. Mono, mono-culture; Co, Co-culture; *, p<0.05; **, p<0.01.

FIG. 7 shows microscopic observation of suspension grade human primary hepatocytes in mono-culture (left) or co-culture with endothelial cells and fibroblasts (right). Hepatocytes in mono-culture started to detach from the day after seeding while the majority of hepatocytes plated in co-culture remained attached in good quality.

FIG. 8 shows suspension grade hepatocytes in co-culture exhibiting similar or higher expression levels of CYP450 1A2 (top) and CYP450 3A4 (bottom) compared to plateable grade hepatocytes in mono-culture. Both plateable and suspension grade hepatocytes showed higher metabolic activity in co-culture compared to mono-culture. Mono-U, mono-culture uninduced; Mono-I, mono-culture induced; Co-U, co-culture uninduced; Co-I, co-culture induced.

FIG. 9 shows suspension grade hepatocytes in co-culture exhibiting similar or higher secretion levels of albumin (left) and urea (right) compared to plateable grade hepatocytes in mono-culture. Both plateable and suspension grade hepatocytes showed higher functional albumin and urea secretion in co-culture compared to mono-culture. Mono-U, mono-culture uninduced; Mono-I, mono-culture induced; Co-U, co-culture uninduced; Co-I, co-culture induced.

FIG. 10 shows human primary hepatocytes maintained for approximately 7 days of culture under conventional sandwich culture condition (mono-culture). Six batches of hepatocytes, including three batches of plateable grade hepatocytes and three batches of suspension grade hepatocytes, were observed over 4 weeks of culture. The majority of the hepatocytes detached around day 7 of culture.

FIG. 11 shows hepatocytes plated onto a mixture of endothelial cells and fibroblasts in co-culture maintained up to 6 weeks. 10× magnification.

FIG. 12 shows immunocytochemical staining of hepatocytes 43 days in co-culture after plating. Staining of albumin (bottom right) and CD31 (bottom left) indicated geographical distribution of hepatocytes, endothelial cells and fibroblasts. 10× magnification.

FIG. 13 shows hepatocytes exhibiting higher activity level of CYP450 3A4 in co-culture (upper solid line) compared to those in mono-culture (lower dashed line) over 6 weeks of culture. The activity level of CYP450 3A4 was peaked at week 3 and sustained metabolic activity for 6 weeks in co-culture whereas that in mono-culture discontinued metabolic activity. **, p<0.01.

FIG. 14 shows functional bile secretion from human hepatocytes in co-culture observed by imaging CDFDA efflux to bile canaliculi. 10× magnification.

FIG. 15 shows hepatocytes from three separate batches and pooled those 3 batches in co-culture condition maintained up to 6 weeks. Hepatocytes were overlayed with MATRIGEL on next day after seeding on a mixture of endothelial cells and fibroblasts. 10× magnification.

FIG. 16 shows that hepatocytes pooled from three different batches could be maintained up to 6 weeks in in co-culture. Hepatocytes were cultured without overlayed MATRIGEL on a mixture of endothelial cells and fibroblasts. 10× magnification.

FIG. 17 shows secretion levels of albumin (left) and urea (right) from hepatocytes in co-culture without tapping MATRIGEL was similar to hepatocytes in co-cultured with MATRIGEL tapping. MG, MATRIGEL tapped; No MG, Non MATRIGEL tapped; D4, day 4; D7, day 7.

FIG. 18 shows expression levels of CYP450 3A4 from hepatocytes in co-culture without tapping MATRIGEL similar to hepatocytes in co-cultured with MATRIGEL tapping up to 6 weeks. MG, MATRIGEL tapped; No MG, Non MATRIGEL tapped.

FIG. 19 shows metabolic activity measured by CYP450 3A4 from three different batches of human primary hepatocytes similar to that of pooled hepatocytes from the three different batches in co-culture up to 6 weeks.

FIG. 20 shows that endothelial cells of different sources support a hepatocyte culture. Both immortalized liver sinusoidal endothelial cells (SECs) and human umbilical vein endothelial cells (HUVECs) could support a hepatocyte culture.

FIG. 21 shows similar expression levels of CYP450 1A2 and CYP450 3A4 by hepatocytes co-cultured on immortalized liver sinusoidal endothelial cells (SECs) or human umbilical vein endothelial cells (HUVECs). D4, day 4; D7, day 7.

FIG. 22 shows similar levels of albumin and urea secretion by hepatocytes co-cultured on immortalized liver sinusoidal endothelial cells (SECs) or human umbilical vein endothelial cells (HUVECs) showed. D4, day 4; D7, day 7.

FIG. 23 shows morphology of hepatocytes in co-culture at different cell ratios. HH, human hepatocyte; Fb, fibroblast; EC, endothelial cell.

FIG. 24 shows expression levels of CYP450 1A2 and CYP450 3A4 by hepatocytes co-cultured with endothelial cells and fibroblasts at different ratios. The cellular mixture of hepatocyte, endothelial cells, and fibroblasts at 12:1:1 showed highest expression levels of CYP450 1A2 and CYP450 3A4 compared to those at other ratios. CTRL, uninduced control; Ind, Induced.

FIG. 25 shows expression level of sodium taurocholate cotransporting polypeptide (NTCP) as a cellular receptor for hepatitis B virus (HBV) on co-cultured hepatocytes in multiple time points.

FIG. 26 shows geographical distribution of hepatocytes and feeder cells in co-culture. Size of cellular nuclei was measured on Image J software from the entire field of image (left) and hepatocyte cluster (right).

FIG. 27 is schematic diagram of a co-culture system according to one embodiment of the invention.

FIG. 28 shows morphology of hepatocytes from donors A (10 days old), B (3 years old), and C (4 months old) in a co-culture with human feeder cells on days 1 and 14.

FIGS. 29A-B show a hepatic tri-culture with Kupffer cells (KC). (A) Images of primary human hepatocytes (PPHs) co-cultured with human feeder cells and Kupffer cells as stained for fibrotic marker Cytokeratin-18 (CK18) (red), macrophage marker CD68 (green) and nuclear stain DAPI (blue) in absence (left image) or presence (right image) of lipopolysaccharide (LPS). (B) IL-6 secretion by PPHs in a mono-culture or tri-culture with human feeder cells in the absence or presence of KCs and/or LPS.

FIGS. 30a-f show differences in attachment and lipogenesis between normal and diseased PHHs in the hTCS and DhTCS. (a) Representative images of normal and diseased PHHs on days 3 and 14 in the hTCS and DhTCS. (b) Representative images of diseased hepatocyte morphology in the DhTCS on days 3 and 14. (c) The number of PHHs attached and (d) percent confluency were determined in the hTCS and DhTCS on day 14 (Student t-test). (e) Nile Red staining and (f) quantification in the hTCS (gray bars) and DhTCS (black bars) PHHs on days 3 and 14 (One-way ANOVA plus Tukey). Magnification 10×. * p≤0.05, ** p≤0.01, *** p≤0.001 to hTCS PHHs. Error bars represent standard deviation (n≥3 donors per condition).

FIGS. 31a-h show determination of functional differences in hTCS and DhTCS PHHs. Levels of (a) albumin and (b) urea produced by hTCS and DhTCS PHHs on days 3 (gray bars) and 15 (black bars). Gene expression represented by C(t) values of (c) PCK1, (d) G6PC, and (e) FASN on day 15 from hTCS and DhTCS PHHs (Student t-test). (f) Baseline or uninduced CYP3A4 activity in hTCS and DhTCS PHHs on days 3 (gray bars) and 15 (black bars). (g) Representative images of CYP2E1 (red) plus DAPI (blue) from hTCS and DhTCS PHHs on day 15. (h) Quantitation of CYP2E1 expression normalized to DAPI expression in hTCS PHHs (gray bars) and DhTCS PHHs (black bars) on day 15 (Student t-test). For (a), (b), and (f), values were normalized to the number of attached PHHs for each lot. * p≤0.05, *** p≤0.001 to hTCS PHHs. Error bars represent standard deviation (n≥2 donors per condition).

FIGS. 32a-f show determination of macrophage and cytokine expression in normal and diseased PHHs in the hTCS and DhTCS. (a) Gene expression represented as fold change to Feeder Cells (FCs) of CD68 and CD163 in hTCS (gray bars) and DhTCS (black bars) on day 15. (b) Expression of macrophage markers CD68 (green, left column) and CD163 (green, right column) plus DAPI (blue) for nuclear stain in hTCS (top row) and DhTCS (bottom row) on day 15. (c) Quantitation of CD68 and CD163 expression in hTCS (gray bars) and DhTCS (black bars) on day 15. (d) Levels of IL-6 and MCP-1 were measured in hTCS (gray bars) compared to DhTCS (black bars) on day 15. Values were normalized to the number of attached PHHs for each lot tested in each system. (e) Representative images of nuclear stain DAPI (blue) and either IL-6 (red, left column) or MCP-1 (red, right column) in hTCS (top row) and DhTCS (bottom row) on day 15. (f) Quantitation of IL-6 and MCP-1 expression normalized to DAPI expression in hTCS and DhTCS on days 3 (gray bars) and 15 (black bars). Magnification 10×. Student t-test for (a), (c), (d). One-way ANOVA with Tukey for (f). * p≤0.05, ** p≤0.01, p≤0.001 to hTCS on day 15; +p≤0.05, ++p≤0.01 to DhTCS on day 3. Error bars represent standard deviation (n≥2 donors per condition).

FIGS. 33a-d show diseased PHHs show expression of fibrotic markers. Levels of (a) CK-18 and (b) TGF-β were measured in hTCS (gray bars) compared to DhTCS (black bars) on day 15. (c) Representative images of nuclear stain DAPI (blue) and either CK-18 (red, left column) or TGF-β (red, right column) in hTCS (top row) and DhTCS (bottom row) on day 15. (d) Quantitation of CK-18 and TGF-β normalized to DAPI expression in hTCS and DhTCS on days 3 (gray bars) and 15 (black bars) (One-way ANOVA plus Tukey). For (a) and (b), values were normalized to the number of attached PHHs for each lot tested in each system (Student t-test). Magnification 10×. * p≤0.05, *** p≤0.001 to hTCS PHHs on day 15; +++p≤0.001 to DhTCS on day 3. Error bars represent standard deviation (n≥2 donors per condition).

FIGS. 34a-f show treatment of normal and diseased PHHs with oleic acid (320 μM) and high glucose (25 mM) increases lipid expression. Representative images of (a) normal and treated PHHs in the hTCS and (b) diseased and treated PHHs in the DhTCS on days 1, 7, and 14. Representative images of Nile Red staining in (c) normal and treated and (d) diseased and treated PHHs in the hTCS and DhTCS on days 1 and 14. (e) Quantification of Nile Red Staining in Relative fluorescent units (RFUs) in untreated (Unt) and treated (Trt) normal and diseased PHHs in the hTCS and DhTCS on days 1 (gray bars) and 14 (black bars). (f) Percent increase in lipid expression measured by Nile Red staining in normal and diseased PHHs without (Untrt) and with treatment (Trt) on days 1 (gray bars) and 14 (black bars) in the hTCS and DhTCS. Magnification 10×. * p≤0.05, *** p≤0.001 to untreated PHHs on day 14. +p≤0.05 to untreated PHHs on day 1. #p≤0.05 to normal treated PHHs on day 14. One-way ANOVA with Tukey for stats. Error bars represent standard deviation (n=2 donors for hTCS; n=1 donor for DhTCS).

FIGS. 35a-d show treatment of PHHs with oleic acid (320 μM) and high glucose (25 mM) decreases functionality in normal and diseased PHHs. (a) Levels of albumin measured on days 1, 7, and 14 in normal and diseased PHHs in the hTCS and DhTCS without treatment (untreated, gray bars) and with treatment (black bars). Values were normalized to total protein concentration for each sample in each condition. (b) Induced CYP3A4 activity measured in normal and diseased PHHs in the hTCS and DhTCS on days 1, 7, and 14 in untreated (gray line) and treated (black line) PHHs. Gene expression of (c) FASN and (d) PCK1 in normal and diseased PHHs in the hTCS and DhTCS on days 1, 7, and 14 without treatment (Untrt, gray bars) and with treatment (black bars). * p≤0.05, ** p≤0.01, *** p≤0.001 to untreated PHHs on specified day. One-way ANOVA with Tukey for stats. Error bars represent standard deviation (n=2 donors for normal donors for hTCS; n=1 for diseased donors for DhTCS).

FIGS. 36a-c show adding Obeticholic acid (OCA, 0.5 μM) to oleic acid (320 μM) and high glucose (25 mM) treatment decreases lipid expression. (a) Representative phase contrast images and (b) Nile Red staining images of PHHs untreated, untreated with OCA, treated with oleic acid and high glucose, and treated with oleic acid and high glucose plus OCA on days 1, 7, and 14 in the hTCS. (c) Quantitation of lipid expression measured by Nile Red staining in untreated, untreated with OCA, treated with oleic acid and high glucose, and treated with oleic acid and high glucose plus OCA on days 1, 7, and 14 in the hTCS. Magnification 10×. * p≤0.05, *** p≤0.001 to untreated PHHs on specified day. ###p≤0.001 to treated PHHs on day 14. One-way ANOVA with Tukey. Average and standard deviation are shown (n=2 donors per condition).

FIGS. 37a-c show OCA (0.5 μM) increases functionality in PHHs treated with oleic acid (320 μM) and high glucose (25 mM). (a) Bile canaliculi staining in untreated, untreated with OCA, treated with oleic acid and high glucose, and treated with oleic acid and high glucose plus OCA (+OCA) on days 1 and 14 in the hTCS. (b) Levels of albumin produced by untreated and oleic acid/high glucose treated PHHs (Lipo) without (−) or with (+) the addition of OCA on days 1, 7, and 14. Values were normalized to total protein concentration for each sample in each condition. (c) Induced CYP3A4 activity measured in untreated (black bars), untreated with OCA (gray bars), treated with oleic acid and high glucose (Lipo, white bars), and treated with oleic acid and high glucose plus OCA (Lipo+OCA, striped bars) on treatment days 1 and 14. Magnification 10×. *p≤0.05, *** p≤0.001 to untreated PHHs on specified day. #p≤0.05, ###p≤0.001 to treated PHHs on specified day. One-way ANOVA with Tukey. Error bars represent standard deviation (n=2 donors per condition).

FIG. 38 show H&E staining of liver tissue from normal donors and diseased donors listed in Table 1.

FIG. 39 show Masson's Trichrome staining of liver tissue from normal donors and diseased donors listed in Table 1.

FIGS. 40a-d show (a) Gene expression represented as fold change to Feeder Cells (FCs) of IL-6 and MCP-1 in hTCS (gray bars) and DhTCS (black bars) PHHs on day 15 (Student t-test). (b) Representative images of DAPI (blue) nuclear stain and either IL-6 (red, left column) or MCP-1 (red, right column) in hTCS (top row) and DhTCS (bottom row) on day 3. (c) Gene expression represented as fold change to Feeder Cells (FCs) of CK-18 and TGF-β in hTCS (gray bars) and DhTCS (black bars) on day 15 (Student t-test). (d) Representative images of DAPI (blue) nuclear stain and either CK-18 (red, left column) or TGF-β (red, right column) in hTCS (top row) and DhTCS (bottom row) on day 3. Magnification 10×. * p≤0.05 to hTCS. Error bars represent standard deviation (n≥2 donors per condition).

FIG. 41 shows morphology of hepatocytes from disease donor A (NAS score 6), B (NAS score 5), and C (NAS score 6) in a co-culture with human feeder cells on days 3 and 14.

FIG. 42 shows in schematic form the steps leading to liver fibrosis after an hepatocellular injury.

FIG. 43 shows the plate layouts used in an example.

FIGS. 44A and 44B show cell morphology at 200× without Dex after 120 hours with and without KCs.

FIGS. 45A and 45B show cell morphology at 200× with Dex after 120 hours with and without KCs.

FIG. 46 shows results with Dex after 72 hours with and without KCs.

FIG. 47 shows results without Dex after 72 hours with and without KCs.

FIG. 48 shows results with Dex after 120 hours with and without KCs.

FIG. 49 shows results without Dex after 120 hours with and without KCs.

FIGS. 50A and 50B show gene expression results after 24 hours of exposure to TGF for ratios of Kupffer cells to hepatocytes of 1:4 (A) and 1:2 (B).

FIGS. 50-57 show gene expression results after 2, 6, 24, or 96 hours of exposure to TGF for ratios of Kupffer cells to hepatocytes of 1:4 and 1:2.

FIG. 58 shows in schematic form, that the TGF-β synthesis in the liver is done primarily in the non-parenchymal cells.

FIGS. 59 and 60 show together in schematic form how a negative feedback loop of the reduced TGF-β synthesis in the liver may be due to a negative feedback loop via the Smad 7 protein complex.

FIGS. 61A, 61B, 61C, and 61D show LDH assays at 2, 6, 24 and 96 hours.

FIGS. 62A and 62B show cell health as a function of urea production over time for the two ratios of Kupffer cells to hepatocytes.

FIGS. 63A and 63B show brightfield (A) and imaging flow cytometry (IFC) (B) photos of the same cell field for the 1:2 ratio of Kupffer cells to hepatocytes at 96 hours.

FIGS. 64A and 64B show a comparison of the IFC results for anti-CD68 and anti-HNF-4-alpha results after 96 hours of exposure for 1:2 and 1:4 Kupffer cells to hepatocytes ratios.

FIG. 65 shows, in schematic form, how urea is synthesized in the liver.

FIGS. 66A and 66B show at 10×, the co-cultured cells at a 1:2 Kupffer cell to hepatocyte ratio at 24 hours of exposure without (A) the TFG-β and with (B) the TFG-β not added via the LNPs.

FIG. 67 shows immunostaining of tri-cultured cells with KC's after 96 hours of culture at a ratio of KC's to hepatocytes of 1:4.

FIG. 68 shows secretion of MIP1α by tri-cultured hepatocytes and Kupffer cells with and without exposure to PAF and/or ABT-491.

FIG. 69 shows secretion of E-selectin by tri-cultured cultured hepatocytes and Kupffer cells with and without exposure to PAF and/or ABT-491 or LPS and/or ABT-491.

FIG. 70 shows secretion of IL-8 by tri-cultured cultured hepatocytes and Kupffer cells with and without exposure to PAF and/or ABT-491 or LPS and/or ABT-491.

### DETAILED DESCRIPTION OF THE INVENTION

The present invention provides plated hepatocytes and preparation and uses thereof. The invention is based on the discovery that co-culturing human hepatocytes with endothelial cells and fibroblasts from a human tissue source improves plateability, functional stability and culture longevity of the human hepatocytes. The human source may be adult. The human source may be neonatal or fetal.

The term “plated hepatocytes” used herein refers to human hepatocytes attached to a surface or a feeder cell layer, either directly or indirectly. The term terms “plateability” or “plateable” used herein refers to the ability of hepatocytes to attach to a surface such as a plastic or treated surface (e.g., a culture vessel or multiwell plate), for example, within a predetermined period of time (e.g., 0.5, 1, 2, 3, 4, 5, 6 or 12 hours) upon exposure of the hepatocytes to the surface. The plateability of hepatocytes may be characterized by a percentage of the cells capable of being attached to the surface, directly or indirectly via, for example, feeder cells or non-cell substances (i.e., substances that are not cells), within a predetermined period of time.

The term “feeder cells” used herein refers to cells other than hepatocytes that assist the hepatocytes to remain viable and functional. Examples of feeder cells include fibroblasts and endothelial cells. The endothelial cells may be derived from human tissue sources. The fibroblasts may be derived from human tissue sources. The endothelial cells may be derived from adult human tissue sources. The endothelial cells may be derived from neonatal or juvenile human tissue sources. The fibroblasts may be derived from adult human tissue sources. The fibroblasts may be derived from neonatal or juvenile human tissue sources. Non-cell substances may be chemical compounds and/or biological molecules. Examples of non-cell substances include extracellular matrix proteins, hemodynamic flow conditions, paracrine and autocrine factors, adhesion proteins, and polysaccharides.

TruVivo® is an all-human cell-based triculture system (hTCS) that includes cryopreserved primary human feeder cells (FCs) and primary human hepatocytes (PHHs). Available from LifeNet Health.

Supplement A includes DMEM (Dulbecco's Modified Eagle Medium), Vasculife® Basal Medium (Lifeline Cell Technology), fetal bovine serum, vitamins, amino acids, FGF, VEGF (vascular endothelial growth factor), IFG (insulin-like growth factor), hydrocortisone, heparin sulfate, pyruvate, ascorbic acid, glutamine, and high glucose in water. Available from LifeNet Health.

Modified Supplement A is Supplement A without hydrocortisone. It may also be Supplement A with higher or lower hydrocortisone concentrations. Available from LifeNet Health.

Supplement B includes fetal bovine serum, sodium pyruvate, insulin, and dexamethasone (Dex) in DMSO and water. Available from LifeNet Health.

Supplement C includes insulin, transferrin, selenous acid, fatty acid-free bovine serum albumin (BSA), lineolic acid, and dexamethasone in ethanol and water. Available from LifeNet Health.

Modified Supplement C includes Supplement C without dexamethasone. It may also be Supplement C with higher or lower dexamethasone concentrations. Available from LifeNet Health.

Feeder Cell Thawing Medium (FCTM) includes DMEM (Dulbecco's Modified Eagle Medium), fetal bovine serum (FBS), non-essential amino acids, sodium pyruvate, and Supplement A. Available from LifeNet Health.

Human Hepatocyte Thawing Medium (HHTM) includes DMEM, non-essential amino acids, vitamins, inorganic salts, glucose, polyvinylpyrrolidone coated colloidal silica particles, and a buffer system in water. Available from LifeNet Health.

TruVivo® plating medium (TCPM) includes DMEM, amino acids, vitamins, inorganic salts, glucose, and a buffer system in water. Available from LifeNet Health.

TruVivo® Culture Medium (TCCM) includes Williams' E Medium (WEM) amino acids, vitamins, inorganic salts, sodium pyruvate, glucose, polyvinylpyrrolidone coated colloidal silica particles, and a buffer system in water. Available from LifeNet Health.

Vasculife® Basal Medium (Lifeline Cell Technology) includes amino acids, vitamins, organic and inorganic supplements and salts in water.

Hepatocytes may be deemed plateable if at least 70%, 75%, 80%, 85%, 90%, 95%, 99% or 100% of the cells are capable of being attached to a surface, for example, within a pre-determined period of time.

Hepatocytes may be deemed non- or poorly plateable if no more than 70%, 65%, 60%, 55%, 50%, 45% or 40% of the cells are capable of being attached to a surface, for example, within a pre-determined period of time.

The term “hepatocyte cluster” used herein refers to a group of two or more hepatocytes in a 3D structure. At least about 70%, 80%, 90%, 95% or 99% of the hepatocytes in the hepatocytes may be in direct hepatocyte-hepatocyte contact, which may be evidenced by the presence of gap junction proteins (e.g., Connexin 32) or tight-junction associated proteins (e.g., occludin, ZO-1, claudin-1 and claudin-4).

In addition to cell-cell interactions and the presence of cellular junctions, the overall shape and 3D architecture of the hepatocytes in the co-culture system maintain their normal structure and function, including cell signaling pathways and normal gene expression program. The co-culture system sustains the overall 3D architecture and shape of the hepatocytes over time thus enhancing their cell-cell interactions and preventing cell spreading and the loss of the normal hepatic phenotype. In contrast, hepatocytes maintained as mono-cultures, especially at subconfluent conditions, lose their cell shape and architecture over time and begin to spread and become ‘thinner’ (lose their height) in the absence of the surrounding stromal cells and their supporting ECM factors. This deterioration in cell shape with extensive cell spreading the hepatocytes begin to show a corresponding reduction and loss of key functions, change in gene expression program, and altered response to drug compounds.

The term “functional stability” or “functionally stable” used herein refers to the stability of one or more basic hepatocyte functions of hepatocytes over a predetermined period of time. The basic hepatocyte functions include albumin and urea synthesis rates, cytochrome P450 enzyme activity rates, and inducible cytochrome P450 levels. The functional stability of hepatocytes may be characterized by a percentage of a basic function of the cells initially, for example, within an initial period of time after isolation from a donor, for example, during the initial 4 to 6 days, being maintained in culture after the predetermined period of time, for example, subsequent 1, 2, 3, 4, 5 or 6 days or 1, 2, 3, 4, 5, 6, 7, 8, 9 or 10 weeks (e.g., up to 28 or 42 days). Hepatocytes are deemed functionally stable if at least 50%, 60%, 70%, 80%, 90%, 95% or 99% of a basic hepatocyte function of the cells is retained after the predetermined period of time, for example, without more than 5, 10, 15, 20 or 25% deviation.

The term “culture longevity” used herein refers to the lifetime of cultivated hepatocytes that remain viable and functional. Cells attached to the surface are also referred to as plated cells. At least 70%, 75%, 80%, 85%, 90%, 95%, 99% or 100% of the plated cells may remain viable while attached to the surface for a predetermined period of time, for example, 1, 2, 3, 4, 5 or 6 days or 1, 2, 3, 4, 5, 6, 7, 8, 9 or 10 weeks. Viable hepatocytes are functional as evidenced by, for example, production of albumin and urea, and cytochrome P450 enzymatic activity.

The term “hepatocytes” as used herein refers to primary liver cells that have been isolated or obtained from one or more donors and that have not been cultured for a predetermined number of passages (e.g., 0, 1, 2, 3, 4 or 5 passages). The hepatocytes may be primary hepatocytes, which have not been cultured. The term “pooled hepatocytes” as used herein refers to primary hepatocytes isolated from two or more donors and then mixed. The hepatocytes can be selected from donors based on certain genotyping information.

The term “donor” used herein refers to a living mammal having a liver. The mammal may be a human, a cow, a pig, a dog, a cat, a non-human primate, a rodent such as a rat or mouse, a horse, a goat, a sheep, or a deer. The donor may be a human having a healthy liver or a liver with a disease or a condition, for example, microsteatosis, metabolic dysfunction-associated steatohepatitis (MASH), Metabolic dysfunction-Associated Fatty Liver Disease (MAFLD) or hepatitis (e.g., A, B, C, D and E). Hepatocytes from MASH disease liver may express markers such as cyto-keratin 18.

The donor may be a human of any age. The donor may be a juvenile, who is between 28 days old and 18 years old. The donor may be a neonate, who is under 28 days old. The donor may be under 1, 2, 3, 4, 5, 6, 7, 14, 21 or 28 days old, or about 0-7, 0-14, 0-21, 0-28, 7-14, 7-21, 7-28, 14-21, 14-28 or 21-28 days old. The donor may be older than 28 days and younger than 1, 2, 3, 4, 5, 6, 7, 8, 9, 10, 11, 12, 13, 14, 15, 16, 17 or 18 years old, or about 1-18, 2-18, 3-18, 4-18, 5-18, 6-18, 7-18, 8-18, 9-18, 10-18, 11-18, 12-18, 13-18, 14-18, 15-18, 16-18 or 17-18 years old. The hepatocytes from a juvenile are referred to as juvenile hepatocytes. The hepatocytes from a neonate are referred to as neonatal hepatocytes.

The term “endothelial cells” used herein refers to any endothelial cells. The endothelial cells may be primary human cells, for example, human umbilical vein endothelial cells (HUVEC). The endothelial cells may not be liver cells. The endothelial cells can be isolated from umbilical cord, liver, lung, kidney, brain, spleen, lymph node, heart, intestine or other arteries, veins or capillary vessels. The endothelial cells may not proliferate. The endothelial cells may be primary cells or cultured up to 1, 2, 3, 4, 5, 6, 7, 8, 9, 10, 11, or 12 passages. The endothelial cells may not be immortal. The endothelial cells may be iPSC-derived endothelial, endothelial-like cells, or placenta-derived endothelial cells.

The term “stromal cells” used herein refers to cells that can become connective tissue cells. Examples of the stromal cells include fibroblasts.

The term “fibroblasts” used herein refers to any fibroblasts. The fibroblasts may be primary human cells, for example, human dermal fibroblasts. The fibroblast may be isolated from skin, lung, bladder, cornea, or other tissue types. The fibroblasts may not be liver cells. The fibroblasts may not proliferate. The fibroblasts may be primary cells or cultured up to 1, 2, 3, 4, 5, 6, 7, 8, 9, 10, 11, or 12 passages. The fibroblasts may not be immortal.

Feeder cells may be prepared by the inactivation and freezing of cultured dermal fibroblasts and HUVECs at or before seven passages in culture. Inactivation of the feeder cells may be completed with mitomycin C or gamma irradiation and then verified through either PCNA or BrdU or cell counting. Inactive feeder cells may be frozen for the preparation of one plate of co-culture per vial. Each lot of feeder cells may be tested to ensure the quality criteria such as but not limited to cell number, viability, cell doubling time, sterility, and purity. The fibroblasts and endothelial cells used herein may have no or low interferences with the hepatocytes used herein. The term “interference” used herein refers to typical characteristics, for example, biological activities, of hepatocytes. For example, the fibroblasts and endothelial cells may have no more than about 0%, 0.1%, 0.5%, 1%, 2%, 3%, 4%, 5%, 7%, 10%, 20%, 30% or 40% of the albumin secretion, metabolic clearance, induction, transporter activities or other biological activities of the hepatocytes used in the co-culture. The fibroblasts and endothelial cells may have a cell viability of about 70%, 75%, 80%, 85%, 90%, 95%, 99%, or higher. The ratio of fibroblasts to endothelial cells such as HUVECs may be in the range from about 1:10 to about 10:1, for example, about 1:1, 1:2, 2:1 or any other desirable ratio, as evidenced by, for example, the expression of a fibroblast marker (e.g., TE-7) and an endothelial marker (e.g., CD31). The feeder cells may be free of Mycoplasma and endotoxin.

The term “co-culture” as used herein refers to a culture comprising two or more types of cells in a culture medium.

The terms “hepatocyte co-culture,” “hepatocyte co-culture system,” “hepatic tri-culture” and “hepatic tri-culture system” are used herein interchangeably and refer a culture comprising hepatocytes and feeder cells, which are endothelial cells and stromal cells (e.g., fibroblasts), in a culture medium. A cell ratio of the hepatocytes and the feeder cells may be in the range from about 1:40 to about 40:1 while a cell ratio of the fibroblast cells and the endothelial cells in the feeder cells may be in the range from about 1:10 to about 10:1. A cell ratio of the hepatocytes, the endothelial cells and the stromal cells (e.g., fibroblasts) may be from 3:1:1, 6:1:1, 12:1:1 to 24:1:1. The cell ratio of the hepatocytes and the feeder cells and the cell ratio of the stromal cells (e.g., fibroblasts) and the endothelial cells in the feeder cells may be adjusted to improve plateability or longevity of the hepatocytes in the co-culture.

The hepatocytes in a hepatocyte culture, for example, a hepatocyte co-culture or hepatic tri-culture, may form a monolayer, and the resulting hepatocyte culture is a 2D culture. The monolayer may in contact with a surface. The surface may be coated with, for example, an extracellular matrix or hydrogel components. The hepatocytes may be neonatal or juvenile hepatocytes. The hepatocytes may be adult hepatocytes.

The hepatocytes in a hepatocyte culture, for example, a hepatocyte co-culture or hepatic tri-culture, may form a 3D structure, and the resulting hepatocyte culture is a 3D culture. The 3D structure may be in suspension. The 3D structure may be contact with a surface. The surface may be coated with, for example, an extracellular matrix or hydrogel components. The hepatocytes may be neonatal or juvenile hepatocytes. The hepatocytes may be adult hepatocytes.

The hepatocytes in a hepatocyte culture, for example, a hepatocyte co-culture or hepatic tri-culture, may proliferate such that the hepatocytes are expanded and an expanded hepatocyte culture is obtained. The hepatocytes in an expanded hepatocyte culture may be neonatal or juvenile or adult hepatocytes. For example, the hepatocytes in an expanded hepatocyte culture may be juvenile hepatocytes. For example, the hepatocytes in an expanded hepatocyte culture may be neonatal hepatocytes. For example, the hepatocytes in an expanded hepatocyte culture may be adult hepatocytes.

The hepatocytes in an expanded hepatocyte culture, for example, an expanded hepatocyte co-culture or expanded hepatic tri-culture, may proliferate by, for example, at least about 1, 5, 10, 50, 100, 500 or 1,000 times, or about 1-10, 1-50, 1-100, 1-500 or 1-1,000 times, for an expansion culture period, for example, at least about 1, 2, 3, 4, 5, 6, 7, 14, 21, 28, 35, 42 or 49 days, for example, at least about 7 or 42 days, or about 1-7, 1-14, 1-21, 1-28, 1-35, 1-42, 1-49, 7-14, 7-21, 7-28, 7-35, 7-42, 7-49, 14-21, 14-28, 14-35, 14-42 or 14-49 days. The expanded hepatocytes may exhibit a mixed immature and mature phenotype during and after the expansion culture period. The expanded hepatocyte culture, for example, an expanded hepatocyte co-culture or expanded hepatic tri-culture, may be used immediately after expansion or subject to processing steps such as subculture and alteration of culture conditions to facilitate differentiation/acquisition of a mature phenotype, subculture and further expansion, or cryopreservation to preserve for future uses.

An expanded hepatocyte culture, for example, an expanded hepatocyte co-culture or expanded hepatic tri-culture, may be useful for drug or chemical safety testing, discovery of new drugs (e.g., biologics), or other in vitro hepatocyte applications.

In an expanded hepatocyte culture, for example, an expanded hepatocyte co-culture or expanded hepatic tri-culture, the hepatocytes may maintain a neonatal or juvenile phenotype, and represent the corresponding age population of the hepatocytes in testing. In an expanded hepatocyte culture, for example, an expanded hepatocyte co-culture or expanded hepatic tri-culture, the hepatocytes may maintain an adult phenotype, and represent the corresponding age population of the hepatocytes in testing.

The expanded hepatocyte culture, for example, an expanded hepatocyte co-culture or expanded hepatic tri-culture, may be useful in clinical applications for treatment of hepatic insufficiency in, for example, cell transplantation or genetic modification, and may be incorporated into composite products intended for clinical use. Suitable composite products include a scaffold, a hydrogel, a device and/or cells of other types.

In one embodiment, the hepatocytes may be treated with a chemical or drug substance to affect the hepatocyte transporter functions, for example, increasing or decreasing bile secretions, in the hepatocytes. The drug substances may be obeticholic acid (OCA).

The hepatocytes in a hepatocyte culture, for example, a hepatocyte co-culture or hepatic tri-culture, may not proliferate such that a non-expanded hepatocyte culture, for example, a non-expanded hepatocyte co-culture or hepatic tri-culture, is obtained. The hepatocytes in a non-expanded hepatocyte culture, for example, a non-expanded hepatocyte co-culture or hepatic tri-culture, may be neonatal or juvenile hepatocytes. The hepatocytes in a non-expanded hepatocyte culture, for example, a non-expanded hepatocyte co-culture or hepatic tri-culture, may be adult hepatocytes. For example, the hepatocytes in a non-expanded hepatocyte culture, for example, a non-expanded hepatocyte co-culture or hepatic tri-culture, may be neonatal hepatocytes. A non-expanded hepatocyte culture, for example, a non-expanded hepatocyte co-culture or hepatic tri-culture, may be useful for drug or chemical testing, drug discovery, or other in vitro hepatocyte applications. In a non-expanded hepatocyte culture, for example, a non-expanded hepatocyte co-culture or hepatic tri-culture, the hepatocytes may maintain a neonatal or juvenile phenotype, and enzymatic functional characteristics of the corresponding donor immediately after isolation to enable age-/donor-specific assessment of compound/chemical/treatment efficacy and safety. In a non-expanded hepatocyte culture, for example, a non-expanded hepatocyte co-culture or hepatic tri-culture, the hepatocytes may maintain an adult phenotype, and enzymatic functional characteristics of the corresponding donor immediately after isolation to enable age-/donor-specific assessment of compound/chemical/treatment efficacy and safety.

The hepatic tri-culture may further comprise macrophages or hepatic non-parenchymal cells (NPCs). The macrophages may be obtained from liver, adipose, bone marrow, lymph nodes, placenta, skin, kidney, spleen or any other tissue source. The macrophage may be used after primary isolation or culture expansion. The macrophage may be liver-derived macrophages, for example, Kupffer cells. The hepatic non-parenchymal cells (NPCs) may be stellate cells or liver endothelial cells. The macrophages may be derived from monocytes. The monocytes may be isolated from blood or other tissue sources, culture expanded and induced into macrophages by various methods. The monocytes may be added into a hepatocyte co-culture system, in which the hepatocytes are from a diseased liver having, for example, MAFLD, MASH, or ASH.

Resident macrophages or Kupffer cells (KCs) play an important role in the clearance of debris and foreign particles from the sinusoidal blood. Upon activation resulting from bacterial byproducts or drug/metabolite-induced injury, KCs produce reactive oxygen species (ROS) and a number of soluble mediators which act on neighbouring cell types, such as hepatic stellate cells (HSCs), hepatocytes, and liver sinusoidal endothelial cells (LSECs), to modulate their response to different types of hepatocellular injury. KCs can provoke HSC activation via the secretion of cytokines, such as transforming growth factor-β1 (TGF-β1), a potent pro-fibrogenic cytokine that stimulates collagen synthesis and deposition. In addition, HSCs can be sensitized to the effects of TGF-β1 via the activation of toll-like receptor 4 (TLR4), which leads to the down-regulation of the TGF-β1 pseudoreceptor, BMP and activin membrane bound inhibitor (BAMBI). KCs also secrete matrix metalloproteinases (MMPs) and tissue inhibitors of metalloproteinases (TIMPs), which are important factors in extracellular matrix (ECM) remodelling and cytokine signalling. KCs can mediate the acute phase response in hepatocytes and alter nuclear receptor (NR) activation as well as the expression of cytochrome P450 enzymes (CYPs) and transporter proteins, which influence hepatocellular susceptibility to insult. KCs also secrete factors that upregulate adhesion molecules on LSECs (e.g., intercellular adhesion molecule [ICAM] and vascular cell adhesion molecule [VCAM]) and chemotactic cytokines that recruit additional inflammatory cells to the site of injury. Due to the multitude, variety and duration of signalling events involved during different types of liver injuries, KCs can play both helpful or harmful roles in drug-induced liver injury (DILI) by modulating both pro-regenerative and proinflammatory processes.

The macrophages, for example, liver-derived macrophages (e.g., Kupffer cells), or other hepatic non-parenchymal cells (NPCs) (e.g., stellate cells or liver endothelial cells) in the hepatic tri-culture are useful for assessing potential of liver fibrosis upon exposure to lipid nanoparticles or other agents, or fibrogenic potential of pro-inflammatory or pro-fibrotic agents, including infectious agents, hyper nutrition (e.g., high fat and/or high sugar), pro-fibrotic factors, pro-steatotic factors, carcinogens, and pro-inflammatory factors. Suitable markers for the assessment include CK18, TGF-β, LOX (Lysyl Oxidase), MMP1 (Matrix Metalloproteinase 1), WT1 (Wilms' Tumor 1), Col1A1 (collagen type 1), IL-1, IL6, MMP9, MMP12, PPAR-gamma, alpha-SMA, adiponectin Rc; soluble and/or insoluble collagen using SirCOL™ or similar assay; and histologic evidence of collagen deposition including sirius red or trichrome.

The macrophages, for example, liver-derived macrophages (e.g., Kupffer cells), or other hepatic non-parenchymal cells (NPCs) (e.g., stellate cells or liver endothelial cells) may be from a donor. The donor may be a human. The human may be healthy. The human may have suffered from a liver disease or condition, for example, microsteatosis, metabolic dysfunction-associated steatohepatitis (MASH), Metabolic dysfunction-Associated Fatty Liver Disease (MAFLD) or hepatitis (e.g., A, B, C, D and E). Hepatocytes from a MASH disease liver may express markers such as cytokeratin 18 (CK18). The donor may show a pathology, for example, MAFLD, MASH, alcoholic steatohepatitis (ASH), alpha-1-antitrypsin deficiency, Wilson's disease, Hepatitis B, Hepatitis C, Hemochromatosis, hyperoxaluria, autoimmune disease, cancer, obesity, type 2 diabetes, or an inborn error of metabolism. The donor may have suffered from MAFLD with an NAS score of 3+. The pathological state of the donor may have been induced by exposure to a pathogenic stimulus. Exemplary stimuli include infectious agents, hyper nutrition (e.g., high fat and/or high sugar), pro-fibrotic factors, pro-steatotic factors, carcinogens, and pro-inflammatory factors.

The macrophage, for example, liver-derived macrophages (e.g., Kupffer cells), or other hepatic non-parenchymal cells (NPCs) (e.g., stellate cells or liver endothelial cells) may have been pre-treated before being incorporated into a hepatic tri-culture. The macrophage, for example, liver-derived macrophages (e.g., Kupffer cells), or other hepatic non-parenchymal cells (NPCs) (e.g., stellate cells or liver endothelial cells) may have been induced to show an M1 or M2 phenotype.

The macrophage, for example, liver-derived macrophages (e.g., Kupffer cells), or other hepatic non-parenchymal cells (NPCs) (e.g., stellate cells or liver endothelial cells) may be added into a hepatic tri-culture at any stage. The macrophage, for example, liver-derived macrophages (e.g., Kupffer cells), or other hepatic non-parenchymal cells (NPCs) (e.g., stellate cells or liver endothelial cells) may be added at the time when the hepatocytes are seeded, after the hepatic tri-culture is stabilized, or at any point of culture at T0-28 days.

The macrophage, for example, liver-derived macrophages (e.g., Kupffer cells), or other hepatic non-parenchymal cells (NPCs) (e.g., stellate cells or liver endothelial cells) may be added into a hepatic tri-culture at any ratio relative to another cell type in the culture. The ratio of the macrophage, for example, liver-derived macrophages (e.g., Kupffer cells), or other hepatic non-parenchymal cells (NPCs) (e.g., stellate cells or liver endothelial cells) to the hepatocytes may be between about 1:100 and about 1:2, for example, 1:100, 1:80, 1:50, 1:20, 1:10 or 1:2.

A hepatic tri-culture with macrophages, for example, liver-derived macrophage (e.g., Kupffer cells), or other hepatic non-parenchymal cells (NPCs) (e.g., stellate cells or liver endothelial cells) may be useful for evaluating a potential risk of a liver injury, fibrosis, inflammation or steatosis induced by a pharmaceutical substance. The pharmaceutical substance may be a chemical compound, a biological molecule or a combination thereof. The pharmaceutical substance may be a therapeutic for treating or preventing a liver disease or condition, or a pathology in a patient in need thereof. The pharmaceutical substance may be selected from the group consisting of small molecules, antibodies, live viruses (e.g., hepatitis B or C), lipid nanoparticle, viral vectors, oligonucleotides and cells.

A composition comprising cells in a culture medium is provided. The cells may comprise hepatocytes (e.g., human hepatocytes) and feeder cells, which may include endothelial cells and stromal cells (e.g., fibroblasts). The cells may further comprise macrophages, for example, liver-derived macrophages (e.g., Kupffer cells), or other hepatic non-parenchymal cells (NPCs) (e.g., stellate cells or liver endothelial cells). The cells may consist of hepatocytes (e.g., human hepatocytes) and feeder cells, which may include endothelial cells and stromal cells (e.g., fibroblasts). The hepatocytes may be neonatal or juvenile hepatocytes. The hepatocytes may be adult hepatocytes. The hepatocytes may be plateable. The hepatocytes may be functionally stable. The hepatocytes may have culture longevity.

Cells are commonly cultured in a culture medium developed to promote a desirable property (e.g., growth) of the cells. Hepatocytes are typically cultured in hepatocyte plating media within a desirable cell concentration range. Stromal cells (e.g., fibroblasts) are typically cultured in stromal cell media (e.g., fibroblast media) within a desirable cell concentration range. Endothelial cells are typically cultured in endothelial cell media within a desirable cell concentration range. When the hepatocytes are co-cultured with the endothelial cells and the stromal cells (e.g., fibroblasts), the hepatocyte plating media, the stromal cell media (e.g., fibroblast media) and the endothelial cell media will contribute to a co-culture plating media used for plating and/or co-culturing the cells.

To achieve a desirable cell ratio of the hepatocytes to the endothelial cells and the stromal cells (e.g., fibroblasts), the composition of the co-culture plating media can be adjusted. The co-culture plating media may comprise the hepatocyte plating media, the stromal cell media (e.g., fibroblast media) and the endothelial cell media at a volume ratio of about (5-30):(1-5):(1-5), for example, from about 10:1:1 to about 20:1:1. In one embodiment, the co-culturing plating media may have a ratio of the hepatocyte media, the endothelial cell media and the fibroblast at 12:1:1, which means twelve parts of the hepatocyte plating media, one part of the endothelial cell media, and one part of the stromal cell media (e.g., fibroblast media).

The hepatocyte plating media may be DMEM (Dulbecco's Modified Eagle Medium, Gibco 21063-029) supplemented with 10% heat-inactivated FBS (Fetal bovine serum, Gibco 10082-147), 1×NEAA (Non-essential amino acids, Sigma M7145), 1 mM sodium pyruvate (Gibco11360-070), 5 μg/mL of Insulin, and 5 μM Dexamethasone. The hepatocyte culture media may be WEM (William's E Media, Gibco A1217601) supplemented with 10 mM HEPES (Fisher BP299100), 1× Glutamax (Gibco 35050061), 1×ITS-A (Gibco 51300-044), and 10 μM Dexamethasone

The endothelial cell media may be purchased from LifeLine. The endothelial cell media may be VasulLife Basal Medium, 5 ng/ml rh-FGF-b, 50 μg/mL Ascorbic Acid, 1 μg/mL Hydrocortisone, 2% FBS, 10 mM L-Glutamine, 15 ng/mL rh IGF-1, 5 ng/mL rh EGF (Epidermal growth factor), 5 ng/mL rh VEGF (Vascular endothelial growth factor), and 0.75 U/mL Heparin Sulfate.

The fibroblast media may be DMEM high glucose (Gibco 11995-065) supplemented with 10% FBS.

In one embodiment, at least 70%, 75%, 80%, 85%, 90%, 95%, 99% or 100, for example, at least 85%, of the hepatocytes are plateable in the presence of the endothelial cells and the fibroblast cells. In another embodiment, less than 100%, 90%, 80%, 70%, 60%, 50%, 40%, 30%, 20% or 10%, for example, less than 60%, of the hepatocytes may be plateable in the absence of the endothelial cells and the stromal cells (e.g., fibroblasts).

The hepatocytes (e.g., human hepatocytes) in the composition may be plated. At least 70%, 75%, 80%, 85%, 90%, 95%, 99% or 100%, for example, 90%, of the plated hepatocytes may remain viable and functional while attached to the surface for a predetermined period of time, for example, at least 1, 2, 3, 4, 5, 6, 7, 14, 21, 28, 35, 42 or 49 days, for example, at least 7 or 42 days.

A product comprising plated human hepatocytes on a surface is provided. At least 40%, 50%, 60%, 70%, 75%, 80%, 85%, 90%, 95%, 99% or 100, for example, at least 70%, of the plated hepatocytes are in one or more hepatocyte clusters on feeder cells. The feeder cells are endothelial cells and stromal cells (e.g., fibroblasts) and are attached to the surface. At least 70%, 75%, 80%, 85%, 90%, 95%, 99% or 100, for example, at least 90%, of the plated hepatocytes in the one or more hepatocyte clusters may be in direct hepatocyte-hepatocyte contact. The one or more hepatocyte clusters may cover at least 40%, 50%, 60%, 70%, 75%, 80%, 85%, 90%, 95%, 99% or 100, for example, at least 50%, of the surface. The surface may have a shortest diameter of at least 0.5, 1, 2, 3, 4, 5, 6, 7, 8, 9, 10 or 100 mm. The hepatocytes may be neonatal or juvenile hepatocytes. The hepatocytes may be adult hepatocytes.

The product may further comprise macrophages, for example, liver-derived macrophages (e.g., Kupffer cells), or other hepatic non-parenchymal cells (NPCs) (e.g., stellate cells or liver endothelial cells). The macrophages, for example, liver-derived macrophages (e.g., Kupffer cells), or other hepatic non-parenchymal cells (NPCs) (e.g., stellate cells or liver endothelial cells) may be from a donor. The donor may be a human. The human may be healthy. The human may have suffered from a liver disease or condition, for example, microsteatosis, metabolic dysfunction-associated steatohepatitis (MASH), Metabolic dysfunction-Associated Fatty Liver Disease (MAFLD) or hepatitis (e.g., A, B, C, D and E). Hepatocytes from a MASH disease liver may express markers such as cytokeratin 18 (CK18). The donor may show a pathology, for example, MAFLD, MASH, alcoholic steatohepatitis (ASH), alpha-1-antitrypsin deficiency, Wilson's disease, Hepatitis B, Hepatitis C, Hemochromatosis, hyperoxaluria, autoimmune disease, cancer, obesity, type 2 diabetes, or an inborn error of metabolism. The donor may have suffered from MAFLD with an NAS score of 3+. The pathological state of the donor may have been induced by exposure to a pathogenic stimulus. Exemplary stimuli include infectious agents, hyper nutrition (e.g., high fat and/or high sugar), pro-fibrotic factors, pro-steatotic factors, carcinogens, and pro-inflammatory factors. The macrophage, for example, liver-derived macrophages (e.g., Kupffer cells), or other hepatic non-parenchymal cells (NPCs) (e.g., stellate cells or liver endothelial cells) may show an M1 or M2 phenotype. The ratio of the macrophage, for example, liver-derived macrophages (e.g., Kupffer cells), or other hepatic non-parenchymal cells (NPCs) (e.g., stellate cells or liver endothelial cells) to the hepatocytes may be between about 1:100 and about 1:2, for example, 1:100, 1:80, 1:50, 1:20, 1:10 or 1:2.

The product may further comprise a culture medium. The culture medium may be a mixture of a hepatocyte culture medium, an endothelial cell medium and a fibroblast medium. At least 70%, 75%, 80%, 85%, 90%, 95%, 99% or 100, for example, at least 90%, of the plated hepatocytes remain on the surface for at least 3, 4, 5, 6, 7, 14, 21, 28 or 42 days.

The plated hepatocytes may exhibit one or more basic hepatocyte functions of hepatocytes. For example, the plated hepatocytes may produce albumin and/or express cytochrome P450. Where the plated hepatocytes are neonatal or juvenile hepatocytes, the plated hepatocytes may maintain a neonatal or juvenile phenotype, respectively. Where the plated hepatocytes are adult hepatocytes, the plated hepatocytes may maintain an adult phenotype.

The endothelial cells may be human cells. For example, the endothelial cells are human umbilical vein endothelial cells (HUVEC). The endothelial cells may not be liver cells. The endothelial cells may not proliferate. The endothelial cells may be primary cells or cultured up to 3, 4, 5, 6, 7, 8, 9 or 10, for example, 7, passages. The endothelial cells may not be immortal.

The stromal cells (e.g., fibroblasts) may be human cells. The fibroblasts may be human dermal fibroblasts. The fibroblasts may not be liver cells. The fibroblasts may not proliferate. The fibroblasts may be primary cells or cultured up to 3, 4, 5, 6, 7, 8, 9 or 10, for example, 7, passages. The fibroblasts may not be immortal.

In the product, the plated hepatocytes, the endothelial cells and the stromal cells (e.g., fibroblasts) may have any cell ratio that improves the plateability of the hepatocytes, for example, from 3:1:1, 6:1:1, 12:1:1 to 24:1:1

The plated hepatocytes may be obtained from a single, two or more donors. The donor may be a human. The human may be healthy. The human may have suffered from a liver disease or condition, for example, microsteatosis, metabolic dysfunction-associated steatohepatitis (MASH), Metabolic dysfunction-Associated Fatty Liver Disease (MAFLD) or hepatitis (e.g., A, B, C, D and E). Hepatocytes from a MASH disease liver may express markers such as cytokeratin 18 (CK18). The donor may show a pathology, for example, MAFLD, MASH, alcoholic steatohepatitis (ASH), alpha-1-antitrypsin deficiency, Wilson's disease, Hepatitis B, Hepatitis C, Hemochromatosis, hyperoxaluria, autoimmune disease, cancer, obesity, type 2 diabetes, or an inborn error of metabolism. The donor may have suffered from MAFLD with an NAS score of 3+. The pathological state of the donor may have been induced by exposure to a pathogenic stimulus. Exemplary stimuli include infectious agents, hyper nutrition (e.g., high fat and/or high sugar), pro-fibrotic factors, pro-steatotic factors, carcinogens, and pro-inflammatory factors.

A method of preparing plated human hepatocytes is provided. The preparation method comprises applying human hepatocytes to a surface in the presence of feeder cells, which are endothelial cells and stromal cells (e.g., fibroblasts), co-culturing the applied hepatocytes with the feeder cells after the applying step, and forming one or more hepatocyte clusters by the co-cultured hepatocytes on the feeder cells, which are attached to the surface. At least 40%, 50%, 60%, 70%, 75%, 80%, 85%, 90%, 95%, 99% or 100, for example, at least 70%, of the co-cultured hepatocytes are in the one or more hepatocyte clusters. The hepatocytes may be neonatal or juvenile hepatocytes. The hepatocytes may be adult hepatocytes.

The applied hepatocytes may be obtained from a single, two or more donors. The donor may be a human. The human may be healthy. The human may have suffered from a liver disease or condition, for example, microsteatosis, metabolic dysfunction-associated steatohepatitis (MASH), Metabolic dysfunction-Associated Fatty Liver Disease (MAFLD) or hepatitis (e.g., A, B, C, D and E). Hepatocytes from a MASH disease liver may express markers such as cytokeratin 18 (CK18). The donor may show a pathology, for example, MAFLD, MASH, alcoholic steatohepatitis (ASH), alpha-1-antitrypsin deficiency, Wilson's disease, Hepatitis B, Hepatitis C, Hemochromatosis, hyperoxaluria, autoimmune disease, cancer, obesity, type 2 diabetes, or an inborn error of metabolism. The donor may have suffered from MAFLD with an NAS score of 3+. The pathological state of the donor may have been induced by exposure to a pathogenic stimulus. Exemplary stimuli include infectious agents, hyper nutrition (e.g., high fat and/or high sugar), pro-fibrotic factors, pro-steatotic factors, carcinogens, and pro-inflammatory factors.

The applied hepatocytes may have a plateability of less than 70%, 65%, 60%, 55%, 50%, 45% or 40%, for example, less than 60%, on the surface in the absence of the feeder cells. The applied hepatocytes may have a plateability of at least 70%, 75%, 80%, 85%, 90%, 95%, 99% or 100%, for example, at least 85%, on the surface in the absence of the feeder cells.

The one or more hepatocyte clusters may cover at least 40%, 50%, 60%, 70%, 75%, 80%, 85%, 90%, 95%, 99% or 100, for example, at least 50%, of the surface. The surface may have a shortest diameter of at least 0.5, 1, 2, 3, 4, 5, 6, 7, 8, 9, 10 or 100 mm. At least 70%, 75%, 80%, 85%, 90%, 95%, 99% or 100, for example, at least 90%, of the plated hepatocytes in the one or more hepatocyte clusters may be in direct hepatocyte-hepatocyte contact.

At least 70%, 75%, 80%, 85%, 90%, 95%, 99% or 100%, for example, at least 90%, of the plated hepatocytes remain plated, i.e., attached to the surface, in the presence of the endothelial cells and the stromal cells (e.g., fibroblasts) for a predetermined period of time, for example, for at least 1, 2, 3, 4, 5, 6, 7, 14, 21, 28, 35, 42 or 49 days, for example, 7 or 42 days. The hepatocytes may be co-cultured with the endothelial cells and the stromal cells (e.g., fibroblasts) for no more than 10, 9, 8, 7, 6, 5, 4, 3, 2 or 1 day(s), for example, no more than 3 days.

According to the preparation method of the present invention, the endothelial cells and/or the stromal cells (e.g., fibroblasts) may be attached to the surface before, when or after the hepatocytes are attached to the surface. The endothelial cells and/or the stromal cells (e.g., fibroblasts) may be attached to the surface before or during the co-culturing step.

The preparation method may further comprise adding macrophages, for example, liver-derived macrophages (e.g., Kupffer cells), or other hepatic non-parenchymal cells (NPCs) (e.g., stellate cells or liver endothelial cells) to the hepatocyte co-culture or hepatic tri-culture. The macrophages, for example, liver-derived macrophages (e.g., Kupffer cells), or other hepatic non-parenchymal cells (NPCs) (e.g., stellate cells or liver endothelial cells) may be from a donor. The donor may be a human. The human may be healthy. The human may have suffered from a liver disease or condition, for example, microsteatosis, metabolic dysfunction-associated steatohepatitis (MASH), Metabolic dysfunction-Associated Fatty Liver Disease (MAFLD) or hepatitis (e.g., A, B, C, D and E). Hepatocytes from a MASH disease liver may express markers such as cytokeratin 18 (CK18). The donor may show a pathology, for example, MAFLD, MASH, alcoholic steatohepatitis (ASH), alpha-1-antitrypsin deficiency, Wilson's disease, Hepatitis B, Hepatitis C, Hemochromatosis, hyperoxaluria, autoimmune disease, cancer, obesity, type 2 diabetes, or an inborn error of metabolism. The donor may have suffered from MAFLD with an NAS score of 3+. The pathological state of the donor may have been induced by exposure to a pathogenic stimulus. Exemplary stimuli include infectious agents, hyper nutrition (e.g., high fat and/or high sugar), pro-fibrotic factors, pro-steatotic factors, carcinogens, and pro-inflammatory factors. The macrophage, for example, liver-derived macrophages (e.g., Kupffer cells), or other hepatic non-parenchymal cells (NPCs) (e.g., stellate cells or liver endothelial cells) may show an M1 or M2 phenotype. The ratio of the macrophage, for example, liver-derived macrophages (e.g., Kupffer cells), or other hepatic non-parenchymal cells (NPCs) (e.g., stellate cells or liver endothelial cells) to the hepatocytes may be between about 1:100 and about 1:2, for example, 1:100, 1:80, 1:50, 1:20, 1:10 or 1:2.

The preparation method may exclude additional extracellular matrix proteins. The additional extracellular matrix proteins may be selected from the group consisting of collagen matrix, e.g., BioCoat, or rat tail collagen, HuGentra Matrix, MATRIGEL and HuBiogel.

The preparation method may further comprise freezing the plated hepatocytes.

For each preparation method, the prepared plated hepatocytes are provided.

A method of testing a pharmaceutical substance is provided. The method comprises administering a pharmaceutical substance to plated hepatocytes in an amount effective to change a property of the plated hepatocytes. The plated hepatocytes may be in the product of the present invention or prepared according to the method of the present invention. The plated hepatocytes may be neonatal or juvenile hepatocytes. The plated hepatocytes may be adult hepatocytes. The plated hepatocytes may be in a hepatic tri-culture. The hepatic tri-culture may be an expanded hepatic tri-culture. The hepatic tri-culture may be a non-expanded hepatocyte co-culture or hepatic tri-culture. The hepatic tri-culture may comprise macrophages, for example, liver-derived macrophages (e.g., Kupffer cells), or other hepatic non-parenchymal cells (NPCs) (e.g., stellate cells or liver endothelial cells). The pharmaceutical substance may be a chemical compound, a biological molecule or a combination thereof. The pharmaceutical substance may be selected from the group consisting of small molecules, lipid nanoparticles, antibodies, bacteria or derivatives thereof, live viruses (e.g., hepatitis B or C), viral vectors, oligonucleotides and cells. Examples of the bacteria or derivatives thereof include liposomes, plasmid vectors, and cell-derived materials including microvesicles, exosomes, lysates, conditioned media, and secreted proteins.

A method of testing drug metabolism is provided. The method comprises administering an effective amount of a drug to plated hepatocytes. The plated hepatocytes may be in the product of the present invention or prepared according to the method of the present invention. The plated hepatocytes may be adult, neonatal or juvenile hepatocytes. The plated hepatocytes may be in a hepatic tri-culture. The hepatic tri-culture may be an expanded hepatic tri-culture. The hepatic tri-culture may be a non-expanded hepatocyte co-culture or hepatic tri-culture. The hepatic tri-culture may comprise macrophages, for example, liver-derived macrophages (e.g., Kupffer cells), or other hepatic non-parenchymal cells (NPCs) (e.g., stellate cells or liver endothelial cells). The drug may be a chemical compound, a biological molecule or a combination thereof. The plated hepatocytes may be in a culture medium. The term “drug metabolism” as used herein refers to conversion or clearance of a drug. A drug metabolite may be generated. The method may further comprise determining the amount of the drug or the drug metabolite in the plated hepatocytes or the culture medium.

A method of testing drug transport is provided. The method comprises administering an effective amount of a drug to plated hepatocytes. The plated hepatocytes may be in the product of the present invention or prepared according to the method of the present invention. The plated hepatocytes may be neonatal or juvenile hepatocytes. The plated hepatocytes may be adult hepatocytes. The plated hepatocytes may be in a hepatic tri-culture. The hepatic tri-culture may be an expanded hepatic tri-culture. The hepatic tri-culture may be a non-expanded hepatocyte co-culture or hepatic tri-culture. The hepatic tri-culture may comprise macrophages, for example, liver-derived macrophages (e.g., Kupffer cells), or other hepatic non-parenchymal cells (NPCs) (e.g., stellate cells or liver endothelial cells). The drug may be a chemical compound, a biological molecule or a combination thereof. The method further comprises determining the cellular uptake and distribution of the drug in the plated hepatocytes.

A method of testing drug toxicity is provided. The method comprises administering an effective amount of a drug to plated hepatocytes. The plated hepatocytes may be in the product of the present invention or prepared according to the method of the present invention. The plated hepatocytes may be adult, neonatal or juvenile hepatocytes. The plated hepatocytes may be in a hepatic tri-culture. The hepatic tri-culture may be an expanded hepatic tri-culture. The hepatic tri-culture may be a non-expanded hepatocyte co-culture or hepatic tri-culture. The hepatic tri-culture may comprise macrophages, for example, liver-derived macrophages (e.g., Kupffer cells), or other hepatic non-parenchymal cells (NPCs) (e.g., stellate cells or liver endothelial cells). The drug may be a chemical compound, a biological molecule or a combination thereof. The method further comprises detecting a toxic event. The detection of a toxic event may be evidenced by a reduced percentage of remaining viable plated hepatocytes.

A method of preparing a hepatitis B virus (HBV) infected hepatocyte culture model is provided. The method comprises inoculating plated hepatocytes with hepatitis B virus (HBV), and incubating the infected plated hepatocytes for at least 7, 14 or 21 days, for example, 14 days. The HBV infected hepatocyte culture model is prepared. The plated hepatocytes may be in the product of the present invention or prepared according to the method of the present invention. The plated hepatocytes may be neonatal or juvenile hepatocytes. The plated hepatocytes may be adult hepatocytes. The plated hepatocytes may be in a hepatic tri-culture. The hepatic tri-culture may be an expanded hepatic tri-culture. The hepatic tri-culture may be a non-expanded hepatocyte co-culture or hepatic tri-culture. The hepatic tri-culture may comprise macrophages, for example, liver-derived macrophages (e.g., Kupffer cells), or other hepatic non-parenchymal cells (NPCs) (e.g., stellate cells or liver endothelial cells). The method may further compromise determining transcription or expression level of a liver-specific bile acid transporter, for example, sodium taurocholate cotransporting polypeptide (NTCP), of hepatocytes, selecting a batch of hepatocytes with a desirable transcription or expression level of NTCP, plating the selected batch of hepatocytes according to the method of current invention before inoculating the plated hepatocytes with hepatitis B virus (HBV). An overlay of protein matrix may not be needed with the current invention to increase the virus inoculation efficiency. A suspension grade hepatocyte batch with a desirable NTCP level may be used for HBV inoculation and suitable for long-term culture studies.

A kit for plating hepatocytes according the present invention is provided. The kit comprises a first cryovial comprising primary hepatocytes, a second cryovial comprising endothelial cells, a third cryovial comprising stromal cells (e.g., fibroblasts), and an instruction for preparing plated human hepatocytes with the primary hepatocytes, the endothelial cells and the stromal cells (e.g., fibroblasts) according to the method of the present invention. The second cryovial and the third cryovial may be the same. According to another embodiment, the feeder cell, i.e., the endothelial cells and the stromal cells (e.g. fibroblasts) may be provided together in a single vial. The first cryovial, second cryovial and the third cryovial may be the same. The primary hepatocytes may be neonatal or juvenile hepatocytes. The primary hepatocytes may be adult hepatocytes. The kit may comprise hepatocyte cryovial from a single donor, or hepatocyte cryovials from multiple donors. The kit may further comprise a fourth cryovial comprising macrophages, for example, liver-derived macrophages (e.g., Kupffer cells), or other hepatic non-parenchymal cells (NPCs) (e.g., stellate cells or liver endothelial cells). The kit comprises test results or certificate of analysis (COA) for the hepatocyte co-culture system. The kit comprises a user instruction protocol for preparation methods. The kit comprises culture mediums for thawing, plating and culturing the hepatocytes, stromal cells (e.g., fibroblasts) and endothelial cells.

A method for preparing a hepatocyte co-culture system is provided. The method comprises isolating hepatocytes from a liver of a donor, adding stromal cells (e.g., fibroblasts) and endothelial cells to the isolated hepatocytes, and freezing hepatocytes mixed with stromal cells (e.g., fibroblasts) and endothelial cells. The hepatocytes may be neonatal or juvenile hepatocytes. The hepatocytes may be adult hepatocytes. The method may further comprise growing cells in culture. The cells may comprise the hepatocytes, the stromal cells (e.g., fibroblasts) and the endothelial cells. The cells may further comprise macrophages, for example, liver-derived macrophages (e.g., Kupffer cells), or other hepatic non-parenchymal cells (NPCs) (e.g., stellate cells or liver endothelial cells). The cells may consist of the hepatocytes, the stromal cells (e.g., fibroblasts) and the endothelial cells. The cells may comprise the hepatocytes, the stromal cells (e.g., fibroblasts), the endothelial cells and macrophages, for example, liver-derived macrophages (e.g., Kupffer cells), or other hepatic non-parenchymal cells (NPCs) (e.g., stellate cells or liver endothelial cells). The donor may be an animal, preferably a human. In one embodiment, all of the hepatocytes, the stromal cells (e.g., fibroblasts) and the endothelial cells are derived from human. In another embodiment, the hepatocytes are derived from an animal species such as rat, mouse, pig, dog, guinea pig, or monkey, and the stromal cells (e.g., fibroblasts) and the endothelial cells are from human. In another embodiment, all of the hepatocytes, the stromal cells (e.g., fibroblasts) and the endothelial cells are derived from an animal species such as rat, mouse, pig, dog, guinea pig, or monkey.

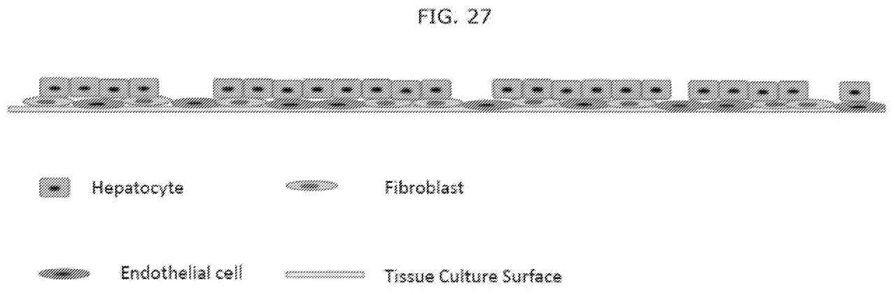

A ready-to-use hepatocyte co-culture system is provided. The co-culture hepatocyte system comprises a tissue culture plate with a culture well or culture wells with or without ECM substrate coating, a fibroblast and endothelial cell layer attached to the surface of the culture well, and a hepatocyte layer attached on the stromal cells (e.g., fibroblasts) and endothelial cells or cell layer, forming hepatocyte clusters or hepatocyte islands. The hepatocyte clusters, also known as hepatocyte islands, are self-assembled after seeding onto or with the stromal cells (e.g., fibroblasts) and endothelial cells. The hepatocytes within the hepatocyte clusters or hepatocyte islands maintain direct hepatocyte-hepatocyte contact. And the hepatocytes produce hepatocyte-hepatocyte adhesion molecules such as cadherins and connexins. The hepatocytes may be neonatal or juvenile hepatocytes. The hepatocytes may be adult hepatocytes. Higher than 70%, 75%, 80%, 85%, 90%, 95% or 99% of the hepatocytes in the co-culture system will distribute in the hepatocyte clusters and maintain direct hepatocyte-hepatocyte contact. Higher than 20%, 25%, 30%, 40%, 50%, 60%, 70%, 75%, 80%, 85%, 90%, 95% or 99% of the surface of the culture well is covered by the hepatocyte clusters. The hepatocytes used herein can be from a single donor, or from multiple donors. And the endothelial cells in the co-culture system may form vessel like structure during culture (FIG. 27). The co-culture hepatocyte system may further comprise macrophages, for example, liver-derived macrophages (e.g., Kupffer cells), or other hepatic non-parenchymal cells (NPCs) (e.g., stellate cells or liver endothelial cells).

### Example 1. Co-Cultured Human Hepatocytes

Human primary hepatocytes co-cultured with human dermal fibroblasts and endothelial cells were characterized. In this example, suspension grade hepatocytes had a plateability of less than 70% while plateable grade hepatocytes had a plateability of at least 70%.

### 1. Methods

### 1.1 Isolation and Cryopreservation of Human Primary Hepatocytes

Human primary hepatocytes were isolated from human donors and then stored in liquid nitrogen.

### 1.2 Source of Human Dermal Fibroblasts and Endothelial Cells

Human dermal fibroblasts were isolated from adult skin and characterized by TE-7 expression. Human umbilical vein endothelial cells (HUVECs) were isolated from umbilical cord and characterized by CD31 and vascular endothelial (VE)-cadherin expressions. Immortalized human liver sinusoidal endothelial cells (SECs) were purchased from abm Inc. Both dermal fibroblasts and endothelial cells were frozen at passage #0 or #1 in freezing medium containing 10% DMSO (Sigma).

### 1.3 Preparation of Inactive Feeder Cell Layer for Co-Culture

Adult dermal fibroblasts were propagated and grown to confluency in DMEM (Dulbecco's Modified Eagle Medium, Gibco) high glucose supplemented with 10% fetal bovine serum. Both HUVECs and SECs were grown to confluency in endothelial cell proliferation medium purchased from Lonza. Once confluent, cells were passaged by an enzymatic digestion with TrypLE (Gibco) or Trypsin-EDTA (Gibco). Either enzyme was used to detach the cells from the tissue culture surface and create a suspension of the cells, which could be counted. The cells in suspension were then seeded at a lower density by increasing the volume of the cell suspension and transferring the cell suspension to a greater surface area of tissue culture plastic than they were previously seeded. Cells were seeded at a density from 10,000 to 15,000 cells/cm² and passaged once the cells reached confluency, about every five to seven days. Cells were passaged for a maximum of five times before they were mitotically inactivated and frozen.

Because Mitomycin C or Gamma irradiation inactivates cells by causing double strand breaks, Mitomycin C was used to inactive confluent fibroblasts and/or endothelial cells. The inactive cells were mitotically inactive and did not proliferate. Briefly, the cells were rinsed three times with Dulbecco's phosphate-buffered saline (DPBS, Gibco, Paisley, UK), incubated with 10 μg/ml of Mitomycin C (Sigma) for 3 hours at 37° C., and then rinsed three times with DPBS. After inactivation, the fibroblasts and endothelial cells were immediately dissociated with TrypLE for cell counts and then resuspended in a cryopreservation medium at a concentration of 1-2 million cells/ml. The cells were then aliquoted into cryovials, and stored at −80° C. overnight. On the following day, the cells were transferred to liquid nitrogen conditions for long-term storage.

1.4 Plating of Suspension Grade and Plateable Grade Human Hepatocytes onto Inactive Feeder Cells, Mixture of Endothelial Cells and Fibroblasts

Feeder cells consisting of human dermal fibroblasts and endothelial cells were thawed and seeded at 25,000 cells/cm² either onto a collagen-coated (BioCoat, Corning) or no matrix-coated general tissue culture plastic 24- or 96-well plates. The tested feeder cell densities were from 12,500 cells/cm² to 100,000 cells/cm²).

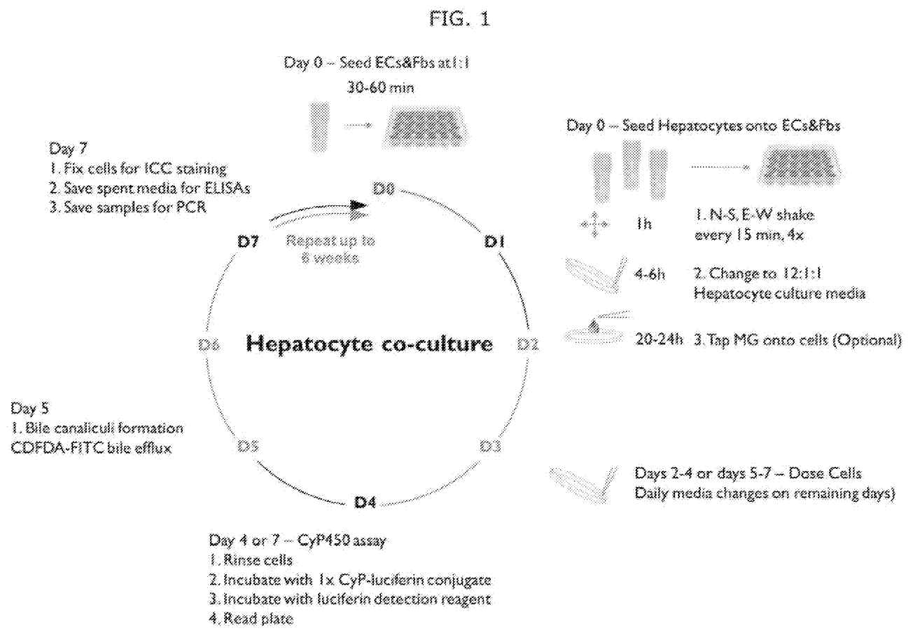

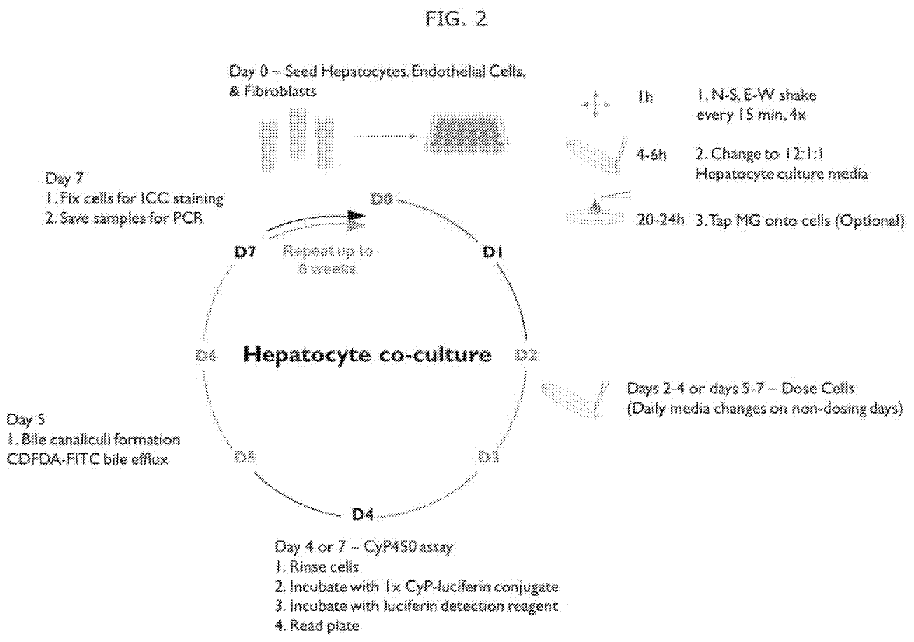

30 minutes after seeding the feeder cells, primary human hepatocytes were seeded at 150,000 cells/cm² (FIG. 1). Traditionally, hepatocytes are seeded at 250,000 cells/cm² onto a collagen coated tissue culture plate. The hepatocyte cellular density may be adjusted in the co-culture system to allow for more or less hepatocyte interaction as well as to account for the attachment rate of the hepatocytes. Alternatively, the feeder cells and hepatocytes can be plated together by mixing the hepatocytes and feeder cells into a homogenous suspension, and then seeding the feeder cells and hepatocytes together onto a collagen-coated plate (FIG. 2). 4-6 hours after seeding the hepatocytes, the medium was changed to remove unattached cells and cellular debris. The incubation time or shaking condition before medium change can vary depending on the type of culture plate (for example, 6-well, 12-well, 24-well, 48-well, 96-well, or 384-well plates) used.

16-20 hours after seeding the hepatocytes, an extra cellular matrix, such as MATRIGEL, was tapped onto the culture for hepatocyte maturation induction. The culture platform was successful without the addition of an extra cellular matrix.

### 1.5 Long-Term Culture of Hepatocytes Under Co-Culture Condition

The traditional plateable hepatocyte monoculture model allows for up to 7 days of culture and analysis of high grade hepatocytes, which begin to spread out due to breakdown of their membranes and end up losing their functionality. The plateable grade hepatocytes and the suspension grade hepatocytes were seeded onto feeder cells in the co-culture model to study their morphology and function up to 6 weeks. The medium was changed daily throughout the culture period. The hepatocyte culture medium contained HEPES (Fisher), GlutaMAX (Gibco), ITS+ (Insulin, transferrin, selenium complex, BSA and linoleic acid, Gibco), dexamethasone (Sigma), and sodium pyruvate (Gibco) in William's E medium (Gibco). In one embodiment, when the hepatocytes are derived from an animal species other than human, the concentration of dexamethasone may be modified (e.g., increased or decreased) to enable the proper biological functions of the hepatocytes to closely represent their functions in vivo. In one example, when the hepatocytes are derived from rat, the concentration of dexamethasone may be reduced.

Throughout the 6 weeks of culture, hepatocytes in co-culture were fed daily with culture medium 12:1:1 HHCM, which is a mixture of hepatocyte culture medium, endothelial cell culture medium, and fibroblast culture medium at a volume ratio of 12:1:1, in studies of their typical cuboidal morphology, bile canaliculi formation, CYP450 enzyme activity, and albumin and urea secretion.

1.6 Pooling Hepatocytes from Multiple Donors Eliminates Donor-to-Donor Variations

Hepatocytes vary in their attachment rate, morphology, protein expression, and enzyme functionality largely due to their donors. Hepatocytes from multiple donors, whether of suspension grade or plateable grade, were pooled to determine if pool could reduce or eliminate some of these variations. Hepatocytes from 3-10 or more different donors were thawed individually, counted, and mixed together in equal amounts. The pooled hepatocytes as well as hepatocytes from each donor were seeded separately onto a feeder layer of fibroblasts and endothelial cells at 150,000 cells/cm². The hepatocytes were pooled together before or after the hepatocytes were frozen.

### 1.7 Characterization of Hepatocytes in Co-Culture Condition

Hepatocytes of suspension or plateable grade were seeded on inactive feeder cells at 25,000 cells/cm² and co-cultured for 6 weeks. Their typical cuboidal and multinucleated hepatocyte morphology was visualized by 5-(and-6)-carboxy-2′, 7′-dichloro-fluorescein diacetate (CDFDA, Invitrogen) efflux in the bile canaliculi throughout the six weeks of culture.

### 1.8 CYP450 Activities of Co-Cultured Hepatocytes

The CYP450 1A2, CYP450 2B6, and CYP450 3A4 activities of the co-cultured hepatocytes were analyzed in Promega's P450-Glo™ assays by following the manufacturer's instructions. Samples were induced for 48 hours by 100 μM Omeprazole for CYP450 1A2, 100 nM CITCO for CYP450 2B6, and 25 μM Rifampicin for CYP450 3A4.

When tested over multiple weeks, the samples were allowed to recover for five days, and then the medium was switched back to the induction medium for another 48 hours before analysis. The activity levels in the induced and uninduced samples were analyzed over six weeks.

### 1.9 Protein and Gene Expression

Spent media samples were collected and evaluated for albumin and urea levels from the co-cultured hepatocytes throughout six weeks of culture. Gene expression levels for albumin, urea, CYP450 1A2, CYP450 2B6, and CYP450 3A4 were assessed weekly throughout the six weeks of culture. The co-culture hepatocytes were fixed after 1 week, 2 weeks, and 6 weeks of culture and stained for albumin, CD31, CD90, CYP450 1A2, CYP450 2B6, and CYP450 3A4.

### 1.10 Bile Canaliculi Formation

The efflux of 5 (and 6)-carboxy-2′,7′-dichlorofluorescein diacetate (CDFDA) was visualized under fluorescence microscope within co-cultured hepatocytes. The co-cultured hepatocytes were rinsed twice with DPBS (—Ca/—Mg), and then incubated with 3:1:1 HHCM at 37° C., 5% CO₂ for 10 minutes before being incubated with the 3:1:1 HHCM containing 5 μM CDFDA at 37° C., 5% CO₂ for 20 minutes. The cells were then rinsed with DPBS (—Ca/—Mg) twice before being imaged in the complete media without phenol red.

### 2. Schematics for Human Hepatocyte Co-Culture on the Mixture of Endothelial Cells and Fibroblasts: Cell Culture and Analysis with Timeframe (FIGS. 1 and 2)

In FIG. 1, human primary hepatocytes were seeded onto a surface pre-plated with a mixture of endothelial cells and fibroblasts as feeder cells (FIG. 1).

30-60 minutes after seeding the feeder cells, the hepatocytes were added to the feeder cells to establish a hepatocyte co-culture having the hepatocytes, the endothelial cells, and the fibroblast cells at a cell ratio of 3:1:1 in a plating medium mixture of the hepatocyte plating medium, the endothelial cell medium, and the fibroblast cell medium at a volume ratio of 3:1:1, also referred to as 3:1:1 HHPM. The co-cultured cells were placed at 37° C., 5% CO₂ and shaken in an N-S and E-W fashion for four times every 15 minutes in the first hour of the hepatocyte co-culture.

4-6 hours after seeding the hepatocytes, the plating medium mixture was changed to a culture medium mixture, of the endothelial cell medium, the fibroblast cell medium and a hepatocyte culture medium at a volume ratio of 3:1:1.

20-24 hours after seeding the hepatocytes, the culture medium mixture became a spent medium and was replaced with fresh mixture of the hepatocyte culture medium, the endothelial cell medium, and the fibroblast cell medium at a volume ratio of 3:1:1, also referred to as 3:1:1 HHCM. If an extracellular matrix such as MATRIGEL was diluted in the culture medium mixture and added to the cells, the culture medium mixture was subsequently replaced daily with fresh 3:1:1 HHCM.

Cytochrome P450 (CYP450) levels were determined in Promega's P450 Glo™ assays on either day four or day seven.

Bile canaliculi were visualized on day 5 of a one-week assay via the efflux of CDFDA. This assay could be repeated multiple times throughout an extended culture.

Protein expression was detected through immunocytochemical (ICC) staining and ELISAs. ELISAs and a colorimetric assay were used to detect and quantify albumin and urea, respectively. The ELISAs were run on 300 μl of the spent media collected after 24 hours of culture. Uninduced and induced samples were fixed for ICC on day 7 and at various time points throughout long-term culture. Cell samples were also snap frozen in Trizol for later RNA extraction and qPCR analysis.

### 2.2 FIG. 2

In FIG. 2, human primary hepatocytes, endothelial cells, and fibroblasts were seeded together (FIG. 2). FIG. 2 is identical to FIG. 1 except that the non-proliferating feeder cells were mixed with human hepatocytes prior to freezing and then revived at the same time, centrifuged, resuspended, and seeded onto a collagen coated tissue culture plastic plate at a density from 100,000 cells/cm² to 775,000 cells/cm² depending on the intended application and the grade of the hepatocytes.

### 3. Co-Cultured Hepatocytes Vs Mono-Cultured Hepatocytes

Human primary hepatocytes were seeded onto feeder cells, a mixture of endothelial cells and fibroblasts, to establish a co-culture under FIG. 1 or FIG. 2, or onto a surface in the absence of the feeder cells to establish a mono-culture. There was no significant difference in the hepatocyte properties (e.g., morphology or gene expression) of the co-cultured hepatocytes prepared under FIG. 2. The co-cultured hepatocytes under FIG. 2 showed better hepatocyte properties (e.g., morphology or gene expression) than mono-cultured hepatocytes.

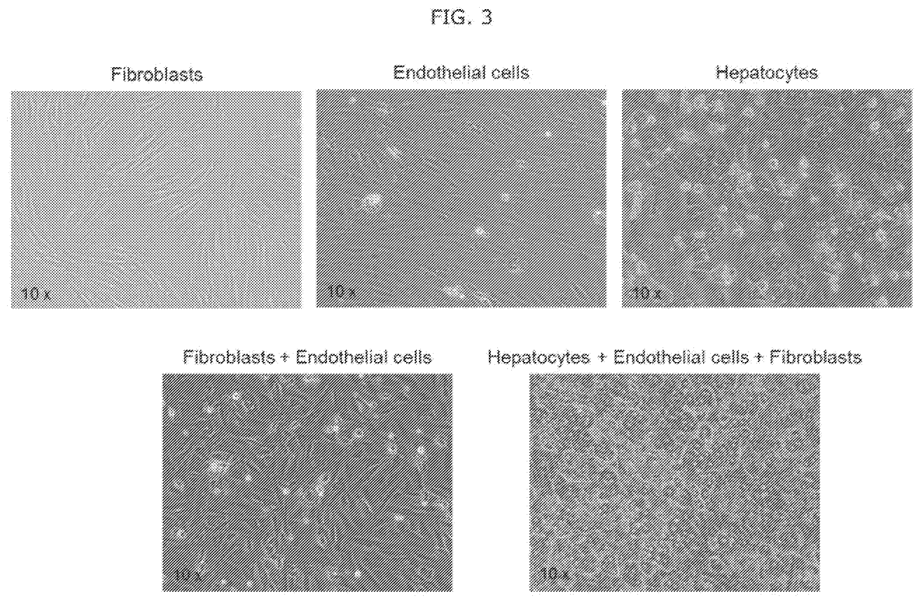

The co-cultured hepatocytes showed better culture longevity and more hepatocyte morphology compared to mono-cultured hepatocytes (FIG. 3).

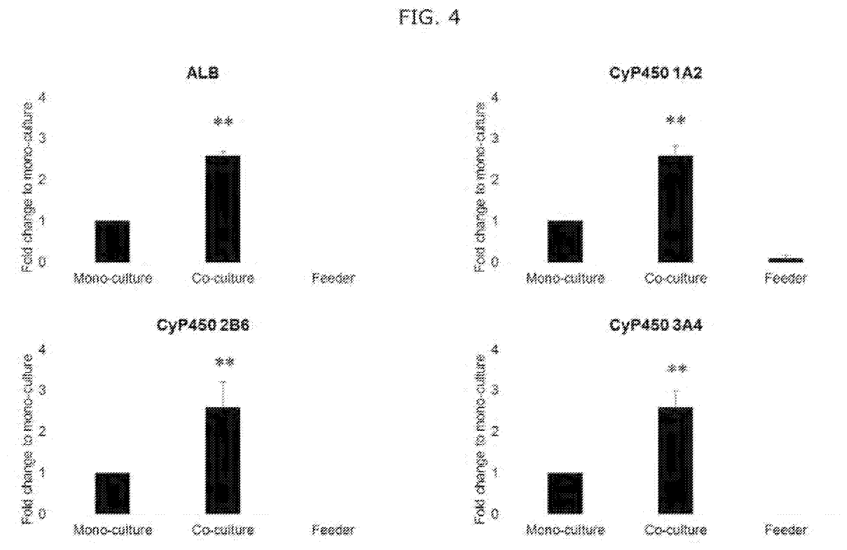

The co-cultured hepatocytes showed significantly higher gene expression of CYP450 1A2, CYP450 2B6, and CYP450 3A4 on day 7 compared to mono-cultured hepatocytes (FIG. 4).

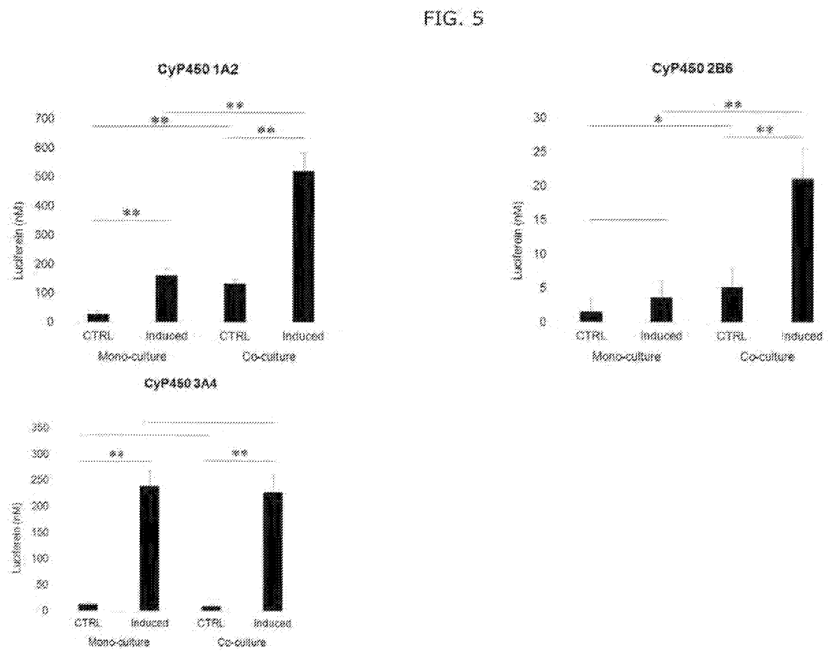

The co-cultured hepatocytes showed much higher CYP450 1A2 activity and CYP450 2B6 activity after induction for 48 hours mono-cultured hepatocytes on day 7. Additionally, on day 7, the co-cultured hepatocytes showed induced CYP450 3A4 activity similar to the mono-cultured hepatocytes (FIG. 5).

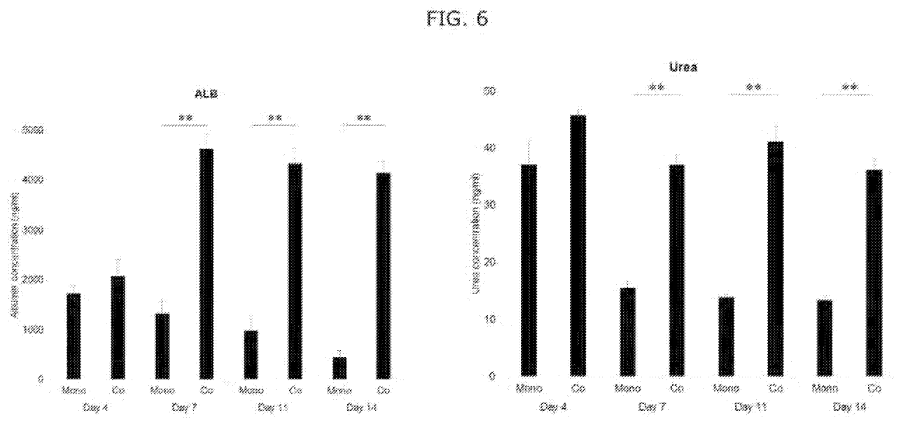

The co-cultured hepatocytes secreted significantly higher levels of albumin and urea compared to mono-cultured hepatocytes over 2 weeks of culture as measured by ELISA (FIG. 6).

### 4. Improved Performance of Co-Cultured Suspension Grade Human Primary Hepatocytes Vs Mono-Cultured Plateable Grade Hepatocytes

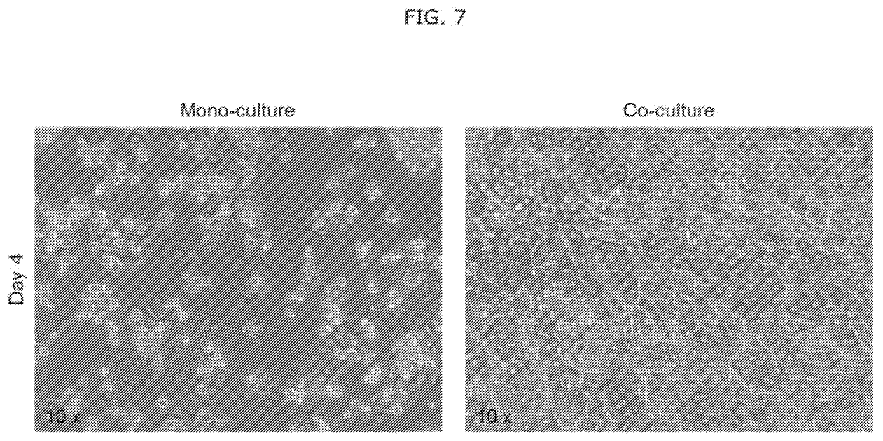

Hepatocytes characterized as a suspension grade are unable to sustain a confluent monolayer for multiple days. Adjusting for the donor's attachment rate with the addition of more cells does not improve the attachment and confluence of the hepatocytes. Even if more hepatocytes are seeded to adjust for the specific donor's attachment rate, it is believed that the additional debris will prevent the cells from attaching. This inability of the suspension grade hepatocytes to attach prevents display of typical hepatocyte morphology or function. The addition of a feeder cell layer consisting of fibroblasts and endothelial cells in a hepatocyte co-culture improved the attachment, morphology, and functionality of the suspension grade hepatocytes (FIGS. 7-9).

The majority of the co-cultured suspension grade human primary hepatocytes remained adherent whereas the majority of the mono-cultured suspension grade human primary hepatocytes never adhered to or detached from a surface by day 4 (FIG. 7).

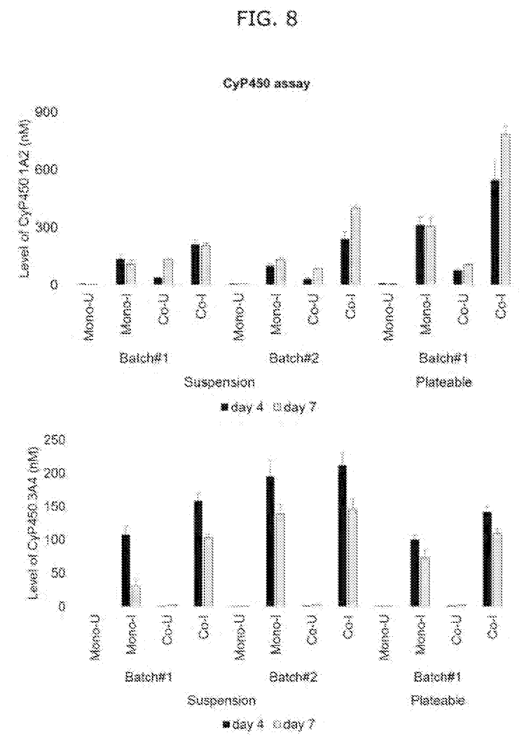

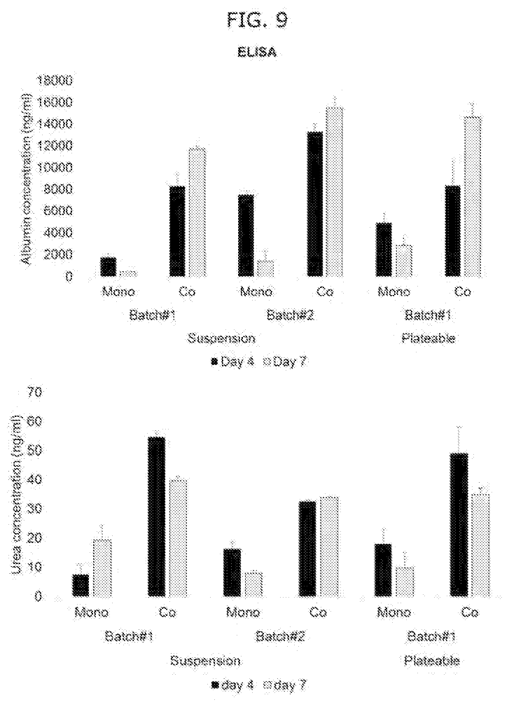

The co-cultured hepatocytes also showed improvements in cellular function over the mono-cultured hepatocytes. Co-cultured suspension grade hepatocytes showed improved CYP450 1A2 activity and CYP450 3A4 activity over the mono-cultured suspension grade hepatocytes on days 4 and 7, and maintained at higher levels through day 7. The CYP450 1A2 activity of the co-cultured suspension grade hepatocytes was similar to that of mono-cultured plateable grade hepatocytes on days 4 and 7. The CYP450 3A4 activity of the co-cultured suspension grade hepatocytes was greater than that of the plateable grade hepatocytes on days 4 and 7 (FIG. 8). Co-cultured suspension grade hepatocytes and the plateable grade hepatocytes showed higher and sustained albumin and urea expression than the mono-cultured suspension grade hepatocytes and the plateable grade hepatocytes as detected by ELISA. The co-cultured suspension grade hepatocytes showed higher albumin and urea expression than the mono-cultured plateable grade hepatocytes on days 4 and 7 (FIG. 9).

### 5. Longevity of Co-Cultured Hepatocytes Morphology, Marker Expression, Metabolic Activity and Functional Bile Secretion

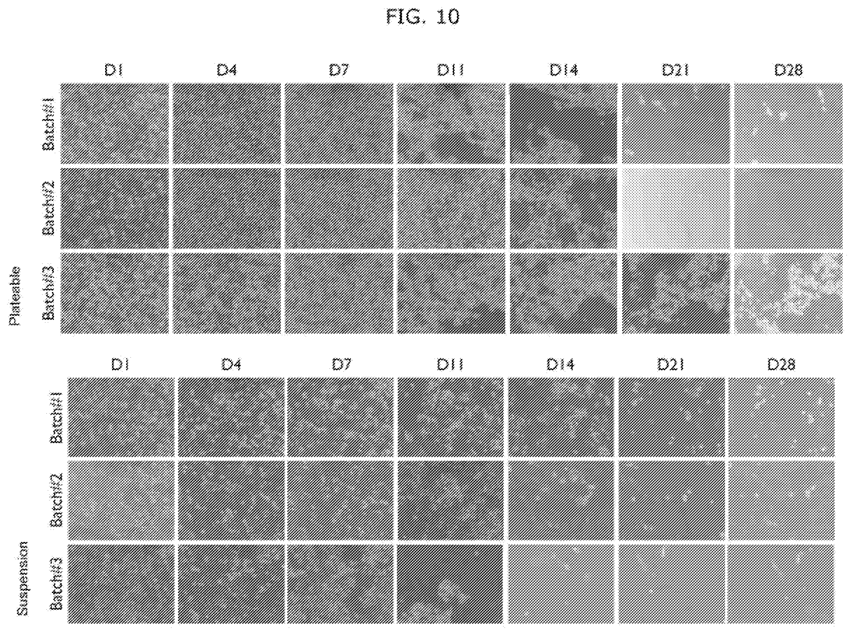

The majority of mono-cultured suspension grade human primary hepatocytes did not attach to a surface, and those that did attach to a surface began to detach from the surface after a maximum of one week in culture. The plateable grade hepatocytes initially attached to a surface at a high efficiency, but began to detach from the surface and lose their cuboidal morphology by day 7 of culture. The plateable hepatocytes did not remain attached for more than 11 days of culture (FIG. 10).

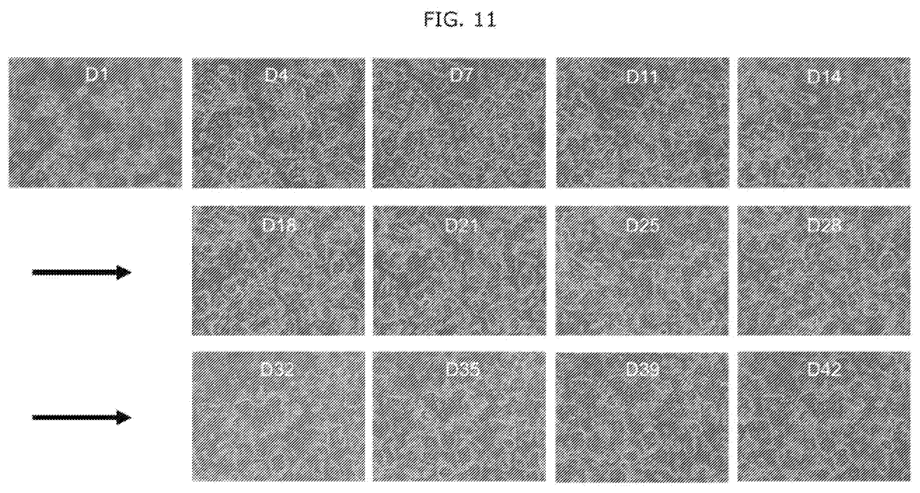

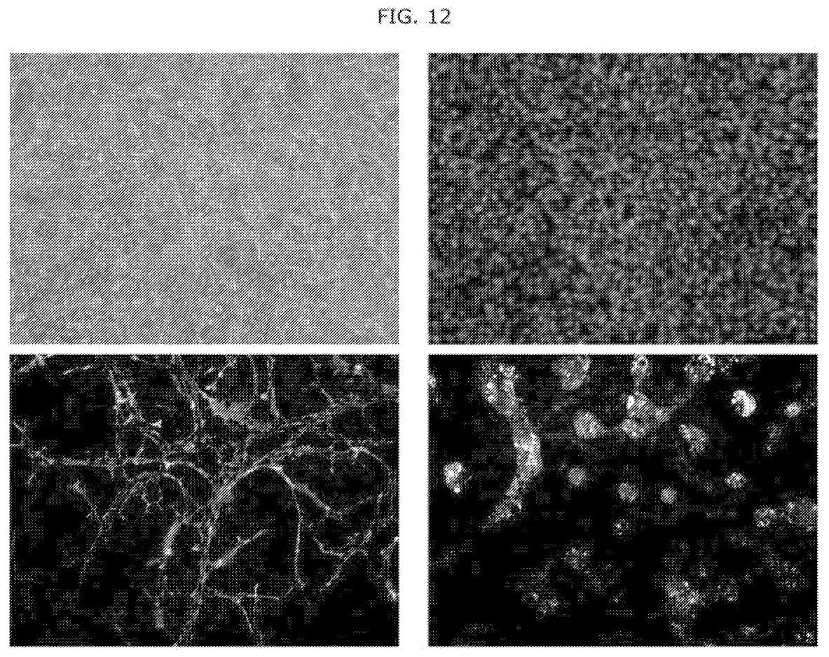

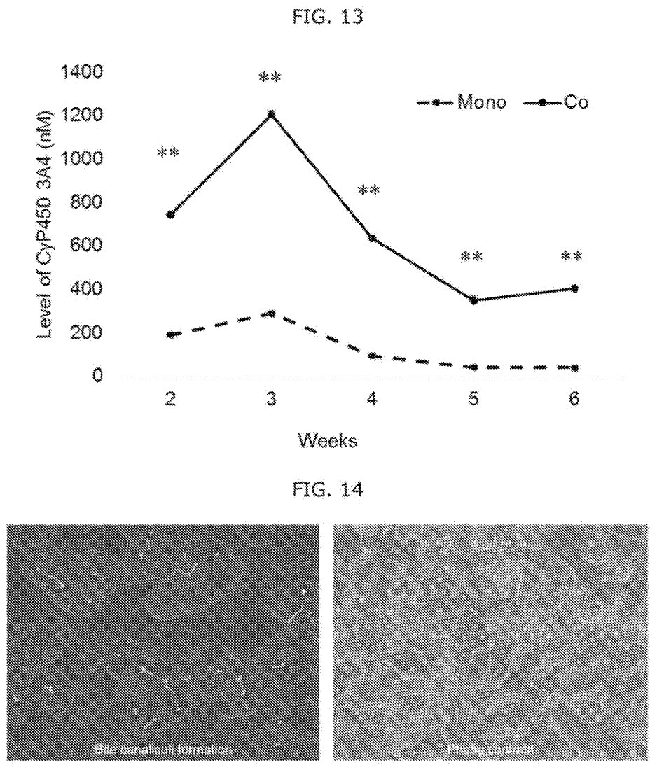

Co-cultured suspension grade hepatocytes were maintained for 6 weeks or longer. The cuboidal hepatocyte morphology was clearly maintained as the hepatocytes that remained attached in compact islands on a non-proliferating feeder layer of fibroblasts and endothelial cells for 6 weeks (FIG. 11). Longer time points were not tested. The interactions between the hepatocytes and the feeder cells were visualized through albumin and CD31 staining. The hepatocytes were integrated into the supporting feeder layer as a complex network of cells. The hepatocyte functions were maintained in this network as visualized by the albumin and CYP450 3A4 staining (FIG. 12). The CYP450 3A4 activity of the co-cultured suspension grade hepatocytes was maintained for 6 weeks. The high activity of the co-cultured suspension grade hepatocytes was in contrast to the low CYP450 3A4 activity of the same hepatocytes cultured as a sandwich monoculture (FIG. 13). The co-cultured suspension grade hepatocytes developed and maintained functional networks of bile canaliculi over the 6 weeks of culture (FIG. 14).

### 6. Longevity of Co-Cultured Hepatocytes: Maturation Induction of Hepatocytes in Co-Culture in the Presence or Absence of MATRIGEL

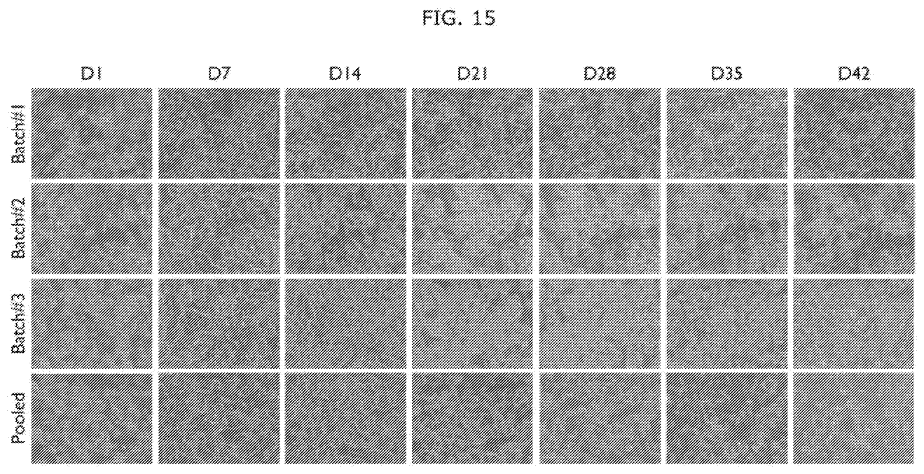

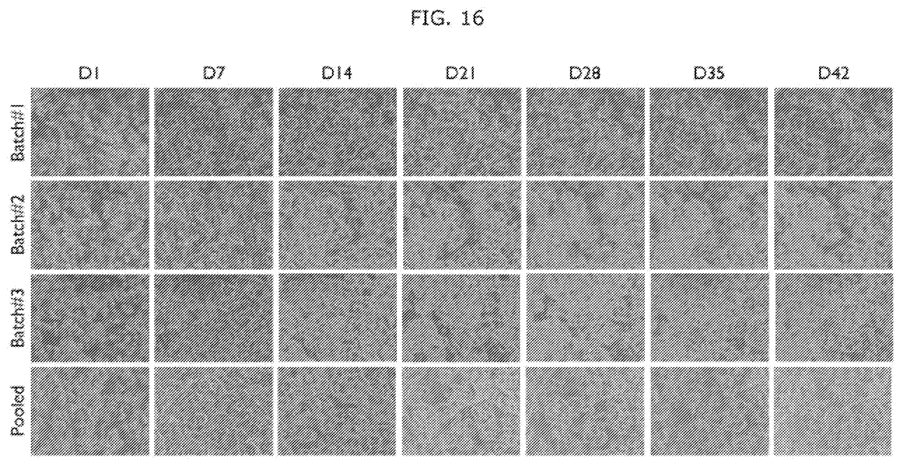

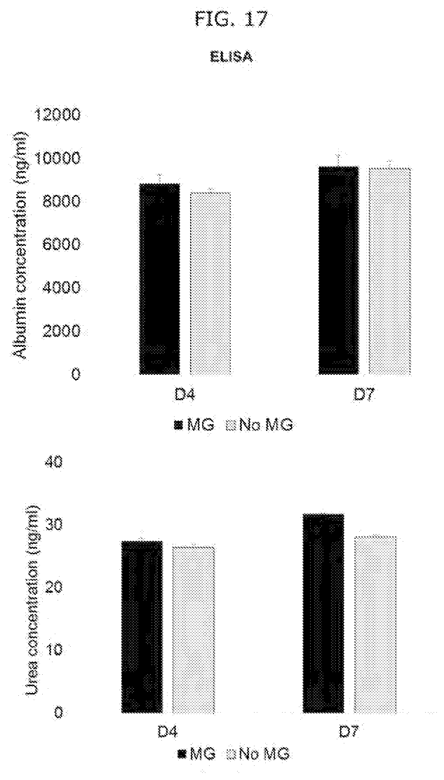

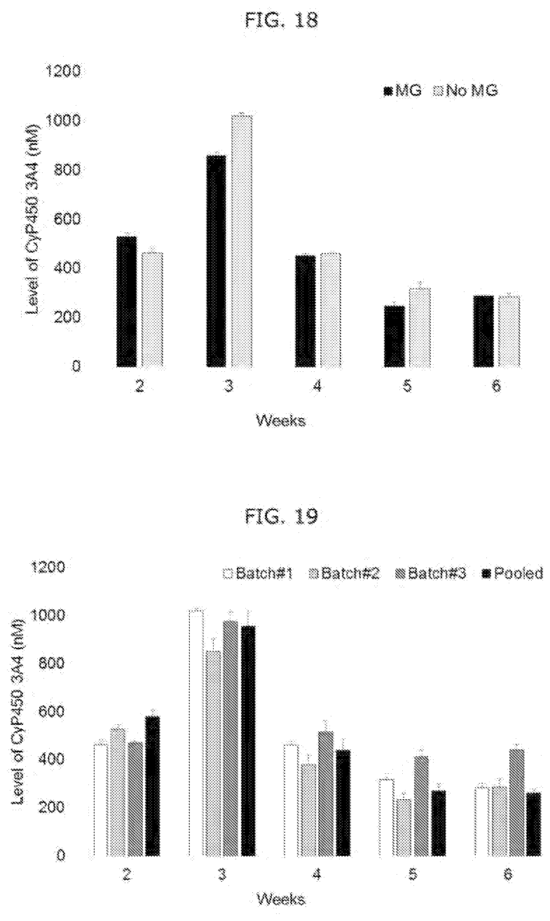

Co-cultured hepatocytes did not need MATRIGEL or any other extracellular matrix to maintain their morphology, function or longevity. The morphology of the co-cultured suspension grade hepatocytes with and without MATRIGEL was remarkably similar throughout weeks 1 through 6 (FIGS. 15 and 16). The Hepatocytes, with and without MATRIGEL, formed compact islands of cells and networks with their feeder layers. The interactions between the feeder cells and the hepatocytes were visualized through ICC after 6 weeks of culture and the bile canaliculi observed throughout the six weeks (data not shown). No morphology difference was observed in the hepatocytes cultured with or without MATRIGEL. The presence of MATRIGEL did not affect the functionality of the co-cultured hepatocytes. Albumin and urea concentrations as well as CYP450 3A4 activity remained constant over the 6 weeks of culture for the co-cultured hepatocytes with and without MATRIGEL (FIGS. 17 and 18).

Mono-cultured and co-cultured hepatocytes from different donors showed significant variations in morphology, protein expression, and enzyme activity. Hepatocytes from multiple donors could be pooled together to normalize these variations. The attachment rate, confluency, and lipid accumulation of pooled hepatocytes from three donors in the co-culture system appeared to be normalized

(FIGS. 15 and 16). The CYP450 3A4 enzyme activity of the pooled hepatocytes appeared to be an average or slightly higher than the average when compared to hepatocytes from individual donors (FIG. 19). The normalization effect of pooling hepatocytes from three or more donors is useful for in vitro testing applications.

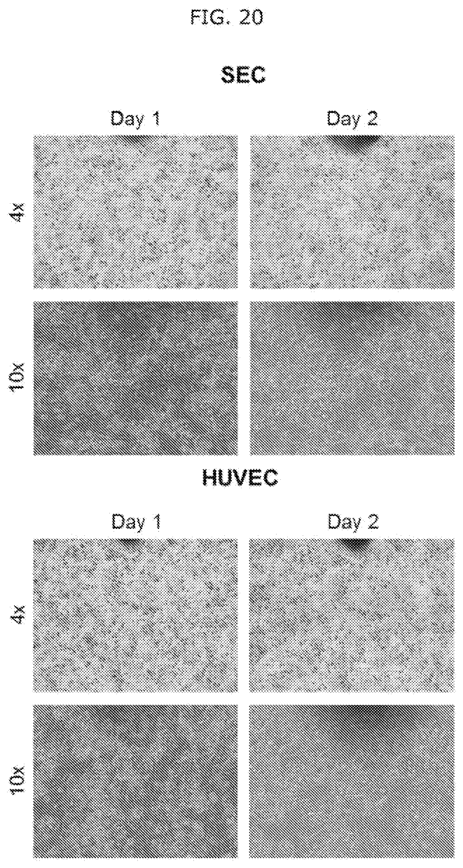

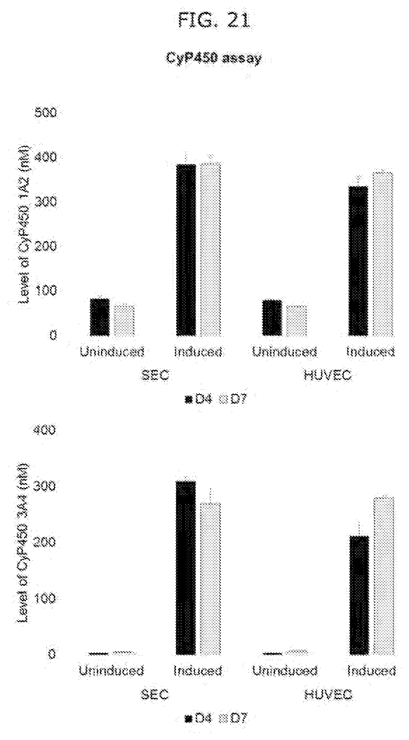

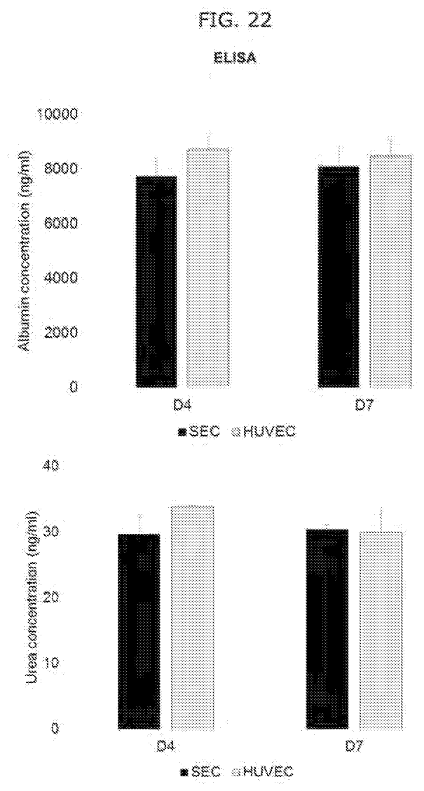

The feeder layer may consist of multiple types of endothelial cells and fibroblasts. The morphology and function of co-cultured human hepatocytes on a feeder cell layer containing sinusoidal endothelial cells or HUVECs were compared. No significant difference was observed in morphology (FIG. 20), CYP450 activity (FIG. 21), or secretion of albumin and urea (FIG. 22).

### 7. Optimal Ratio of Cellular Mixture for Co-Cultured Hepatocytes

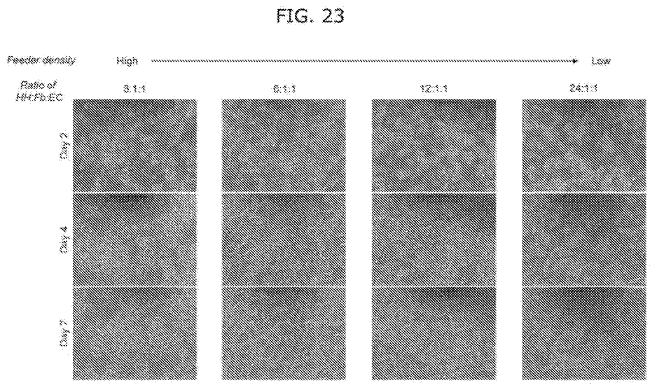

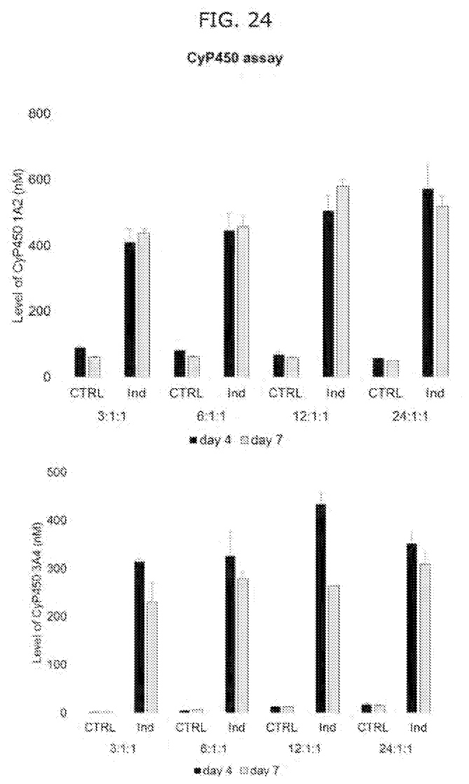

Feeder layer densities from 12,500 cells/cm² to 100,000 cells/cm² were tested in a co-culture system. Although the individual hepatocyte morphology on different feeder cell densities appeared similar, lower densities of feeder cells allowed for more hepatocyte-to-hepatocyte interaction (FIG. 23). The co-cultured hepatocytes showed higher CYP450 1A2 activity and CYP450 3A4 activity at a feeder cell density of 12,500 cells/cm² (24:1:1) or 25,000 cells/cm² (12:1:1) than those at a higher density of 50,000 cells/cm² (6:1:1) or 100,000 cells/cm² (3:1:1) (FIG. 24).

### 8. Multiple Types of Culture Plates for Hepatocyte Co-Culture

Hepatocytes co-cultured on endothelial cells and fibroblasts in either 24- or 96-well plates showed similar features such as morphology, level of albumin secretion, and biomarker expression. Hepatocytes co-cultured on general tissue culture plastic without collagen-coating showed similar morphology and biomarker expression pattern compared to co-cultured hepatocytes on collagen-coated BioCoat plate.

Even though only suspension grade hepatocytes were co-cultured so far, it is expected that the plateable grade hepatocytes will maintain a similar high-grade morphology and function for 6 weeks or longer. It is anticipated that higher grade cells may even develop more complex networks with each other and with the feeder cells more quickly.

### Example 2. HBV Infection Model (NTCP Expression)

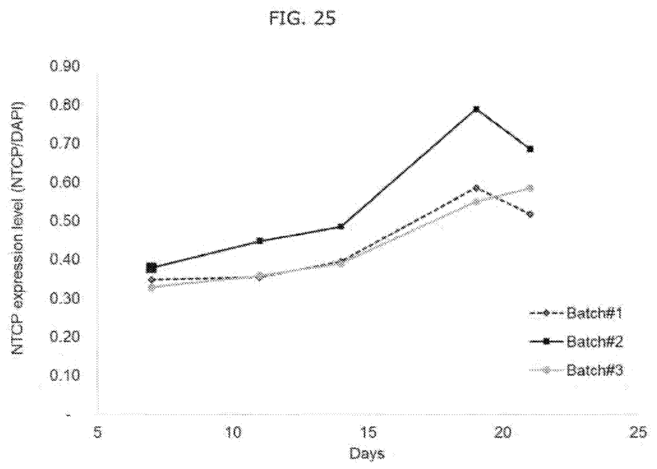

NTCP (sodium/taurocholate cotransporting polypeptide) expression is necessary for the uptake of HBV by hepatocytes. Co-cultured hepatocytes were found to express NTCP on days 7, 11, 14, 19, and 21 detected with immunocytochemistry. Cells were fixed, permeabilized, and incubated with primary antibody SLC10A1 (abcam 131084) at a concentration of 1:100 at 4° C. overnight. Secondary antibody Alexa Fluor 488 goat anti rabbit (Invitrogen A11008) was incubated at a concentration of 1:1000 at 4 C for one hour to detect the primary antibody. Cells were counter stained with Hoechst. NTCP expression was quantified with whole well fluorescence scanning with a BMG CLARIOstar (FIG. 25). Thus, NTCP expression was sustained by the co-cultured hepatocytes over a three-week period.

### Example 3. Optimal Ratios for Cells and Medium

Feeder cell seeding densities of 12,500, 25,000, 50,000, and 100,000 cells per square centimeter were tested for hepatocyte attachment and cluster formation. Hepatocyte seeding densities of 150,000, 250,000, 375,000 cells per square centimeter were tested. Culture media ratios of hepatocyte culture media, endothelial cell media, and fibroblast media were tested for an ideal cellular ratio as well. A feeder cell ratio of 12,500 to 25,000 cells per square centimeter and a hepatocyte seeding density of 150,000 cells per square centimeter showed the highest attachment rate, cluster formation, and hepatocyte function. A medium ratio matching the cellular ratio was also found to maximally support the hepatocyte morphology and function.

### Example 4. Hepatocyte Clusters

A layer of feeder cells composed of fibroblasts and endothelial cells attached to a tissue culture plastic surface within the first minutes after the feeder cells were seeded. The feeder cells were seeded at a density sparse enough to allow the feeder cells to spread out, migrate and coat the tissue culture plastic surface. The hepatocytes then attached onto the top of the feeder cells and migrated towards each other forming clusters hepatocytes. This hepatocyte migration occurred within the first hour or up to 48 hours, depending on the characteristics of the hepatocytes. Once the hepatocyte clusters were formed, adjacent hepatocytes formed functional bile canaliculi and tight junctions. Bile canaliculi was visualized through the efflux of CDFDA starting on day 4. Junctional proteins zonal occludin 1 and connexin-32 were visualized on day 7 using immunocytochemistry. At least 90% of the hepatocytes in the clusters showed tight gap junction, indicating direct hepatocyte-hepatocyte contact.

### Example 5. Cluster Measurements

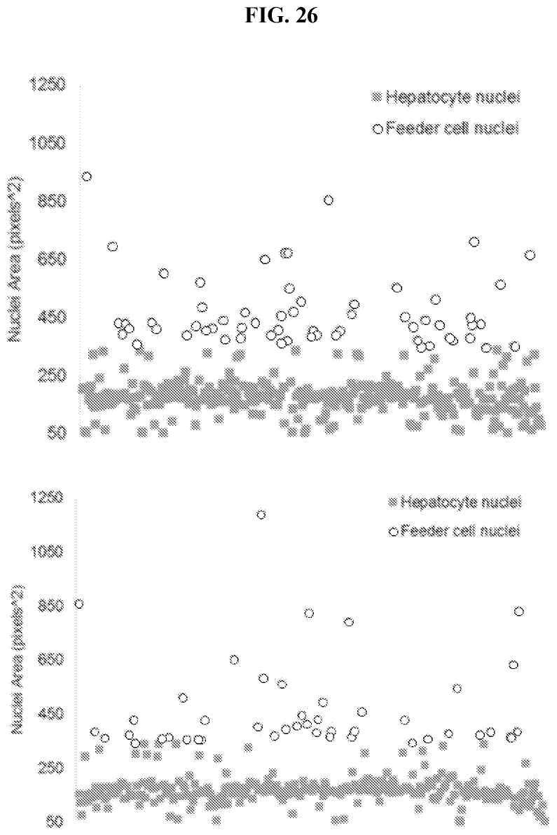

Plateable quality hepatocytes were assessed through analysis of the overall area and nuclei characteristics within the regions not occupied by hepatocyte clusters in comparison to the total cell field. Suspension quality hepatocytes were assessed by analyzing the regions occupied by clusters in comparison with the total cell culture area. The feeder cells and hepatocytes were differentiated by the sizes of their respective nuclei. Fibroblasts and endothelial cells have a larger average nuclear size than hepatocytes. An average nuclear size of the feeder cells was established by measuring the nuclear size of feeder cells alone in the co-culture. A threshold of the average feeder cell area plus one standard deviation was used to distinguish between hepatocytes and feeder cells. The threshold used was a hepatocyte nuclei area less than 345 pixels\hat{ }2. All measurements were taken with ImageJ (FIG. 26).

### Example 6. Co-Culture of Neonatal and Juvenile Hepatocytes

Hepatocytes from donors A (10 day old), B (3 year old), and C (4 month old) were seeded on collagen coated 96-well plates at 20,000-50,000 cells/well with human feeder cells, i.e., fibroblasts and endothelial cells, at 10,000 cells/well. Daily morphology and medium changes were recorded for 14 days of culture. Albumin secretion and urea synthesis were quantitated at days 7, 10, and 14. All data were normalized to attached hepatocytes calculated using image analysis. By day 14, co-cultured hepatocytes from donors A and B expanded while co-cultured neonatal or juvenile hepatocytes from donor C did not expand (FIG. 28).

### Example 7. Hepatic Tri-Culture with Liver-Derived Macrophages (Kupffer Cells)

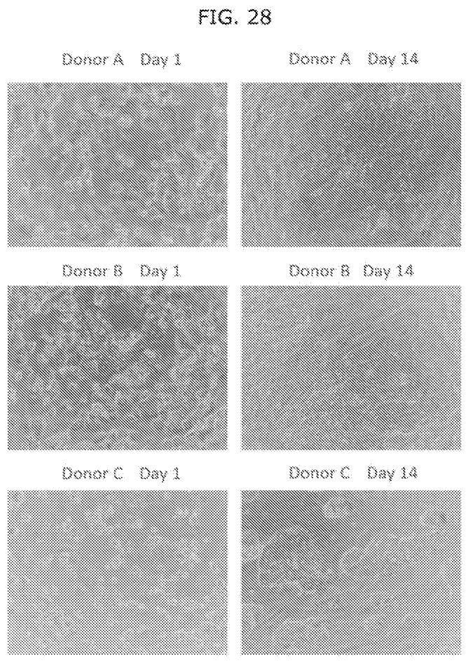

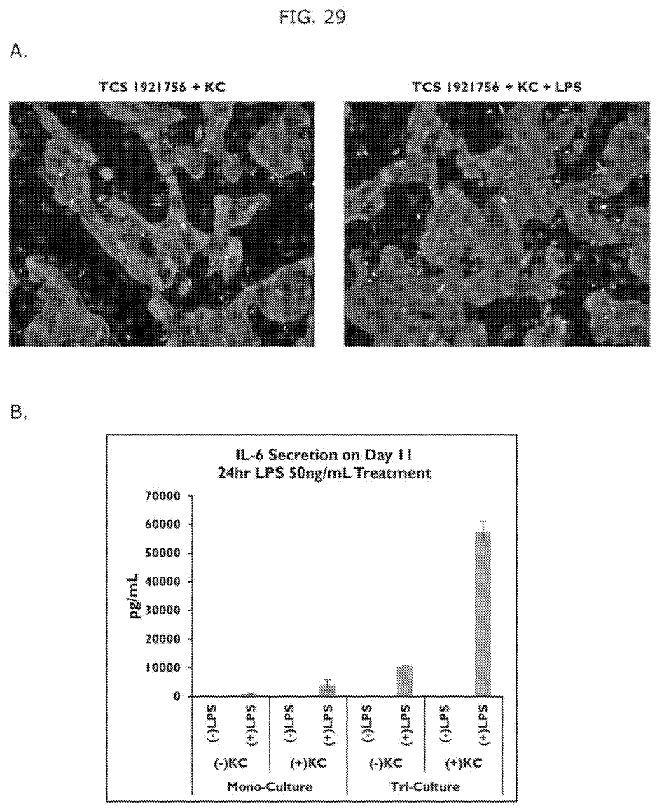

Primary human Hepatocyte (PHH) lot 1921756 was seeded on a collagen coated 24-well plate at 150,000 cells/well with human Feeder Cells (50,000 cells/well). On day 7, Kupffer cell (KCs) lot 2017423 was seeded in the Tri-culture system at 90,000 cells/well. The PHH culture was stained for fibrotic marker Cytokeratin-18 (CK18), macrophage marker CD68, and nuclear stain DAPI on day 11 of culture (FIG. 29A, left image). Lipopolysaccharide (LPS, final concentration 50 ng/mL) was added to the cells for 24 hours of treatment on day 10. On day 11, cells were fixed and stained as described above (FIG. 29A, right image). The level of Interleukin-6 (IL6) from PHHs in the Tri-Culture system on day 11 after 24 hours of LPS treatment (50 ng/ml) was measured by ELISA (FIG. 29B). For the bar graph, the 4 bars to the left are IL6 levels measured from monoculture plated PHHs (150,000 cells/well). The 4 bars to the right are IL6 levels measured from PHHS in the Tri-Culture system. As the bars move from left to right, the two left bars are no KCs added with no LPS treatment (trt) and then with LPS trt. The two right bars are KCs added with no LPS trt and then with LPS trt.

### Example 8. Characterization of Diseased Primary Human Hepatocytes in an all-Human Cell-Based Triculture System

Disease-origin PHHs were applied to the hTCS (DhTCS) to demonstrate the retention of relevant disease features in the model. For these studies, PHHs were selected from tissue donors with known medical history of MAFLD and histopathologic confirmation of disease. Furthermore, a prototypical Farnesoid X Receptor (FXR) agonist Obeticholic Acid (OCA) was applied in the hTCS cultured in a high FFA/high glucose medium (lipogenic medium) to determine the capacity of the hTCS to detect perturbation and/or modulation of key MAFLD-associated biomarkers.

### Morphology and Functionality of Normal Versus Diseased PHHs

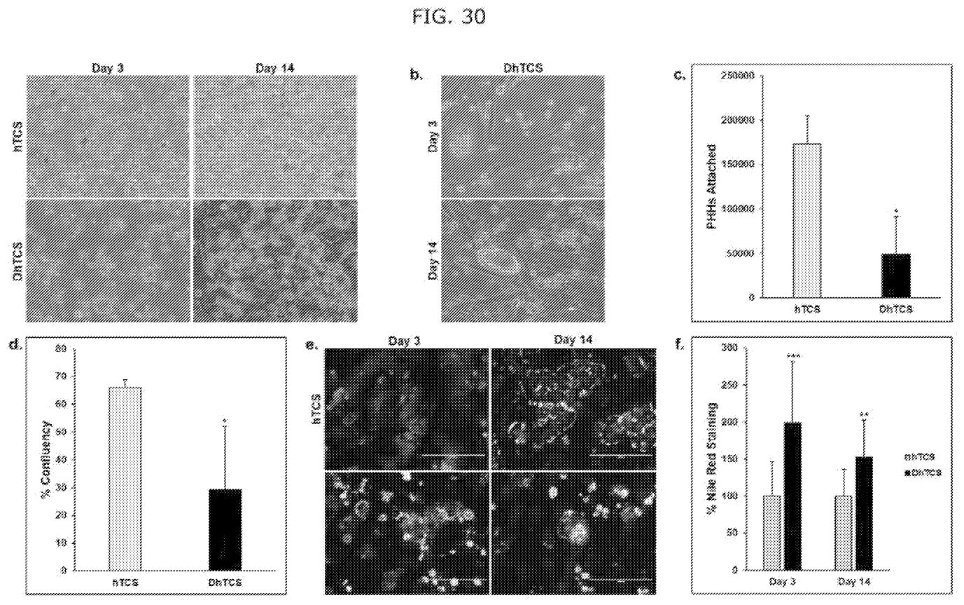

Based on the histopathology of the liver, donors were designated as normal (NAS score ≤3) versus diseased (NAS score of ≥4). Morphological differences were observed between the normal and diseased lots when cultured in the hTCS or DhTCS on days 3 and 14 (FIG. 30). Normal and mild to moderately diseased PHHs had a cuboidal shape and formed hepatocyte colonies (FIG. 30a). Visual differences in hepatocyte morphology were observed in the severely diseased PHHs (FIG. 30b). There were significant differences in PHH attachment (FIG. 30c) and percent confluency (FIG. 30d) between these two groups. Diseased PHHs attached with significantly less efficiency compared to normal PHHs (48,779±42,703 vs 173,614±31,080 PHHs). The confluency was significantly different between normal and diseased PHHs (66.1%±2.7 vs 29.2%±22.9). Diseased PHHs appeared to be more spread out and less compact when compared to normal PHHs and to have greater lipid accumulation compared to normal PHHs (FIG. 30e). Intracellular lipid content was doubled in diseased PHHs on day 3 (198.8%±82.8) compared to normal PHHs (100%±46.4) (FIG. 30f). A decrease in staining was measured from day 3 to 14 (151.9%±50.6) in diseased PHHs; however, they continued to have higher lipid accumulation than normal PHHs (100%±35.9).

The functionality of each group of PHHs was measured and normalized to attached PHHs in the hTCS and DhTCS (FIG. 31). Secretion of albumin (FIG. 31a) and urea (FIG. 31b) were greater in normal PHH lots compared to diseased lots on days 3 and 15. Expression of two genes related to production of enzymes that are critical to gluconeogenesis, PCK1 (FIG. 31c) and G6PC (FIG. 31d), were examined on day 15. Diseased PHHs had lower gene expression of both these genes compared to normal PHHs. The average C(t) value for PCK1 in diseased PHHs was 30.8±2.1 compared to the value in normal PHHs of 28.2±0.7. C(t) values for G6PC were higher in diseased versus normal PHHs (30.6±3.3 vs 27.6±1.4). Gene expression of FASN was also measured, and significantly higher C(t) values were seen in diseased PHHs than in normal PHHs (25.0±1.7 vs 22.4±0.8) (FIG. 31e).

CYP3A4 activity was measured in both diseased and normal PHHs on days 3 and 15 (FIG. 31f). Like albumin and urea activity, uninduced CYP3A4 activity normalized to the number of attached PHHs was lower in diseased PHHs. CYP2E1 expression was determined on day 15 (FIG. 31g). Staining of CYP2E1 was more intense in diseased PHHs compared to normal PHHs on day 15. When expression was quantitated, there was significantly higher CYP2E1 protein expression in diseased PHHs (4,893±3,187 RFUs) versus normal PHHs (542±133 RFUs) (FIG. 31h).

### Diseased PHHs and Inflammation

The link between macrophages and inflammatory markers was examined in diseased and normal PHHs (FIG. 32). The presence of resident macrophages (a normal low-frequency contaminant of PHH preparations) in the hTCS and DhTCS was determined by measuring gene expression of CD68 and CD163 on day 15 (FIG. 32a). A significant fold change difference was seen in CD68 expression between the two systems (diseased fold-change 1.0±0.2 vs normal fold-change 1.3±0.2). CD163 gene expression was significantly higher in the DhTCS (4.9±1.8) compared to the hTCS (3.3±1.2). Immunostaining for CD68 and CD163 was performed in the hTCS and DhTCS on day 15 (FIG. 32b). Quantitation of CD68± and CD163± cells revealed a significantly higher fraction of macrophages in the DhTCS (CD68: 524,578±358,720 and CD163: 299,177±192,429) compared to hTCS (CD68: 7,039±3,788 and CD163: 5,808±2,264) on day 15 (FIG. 32c).

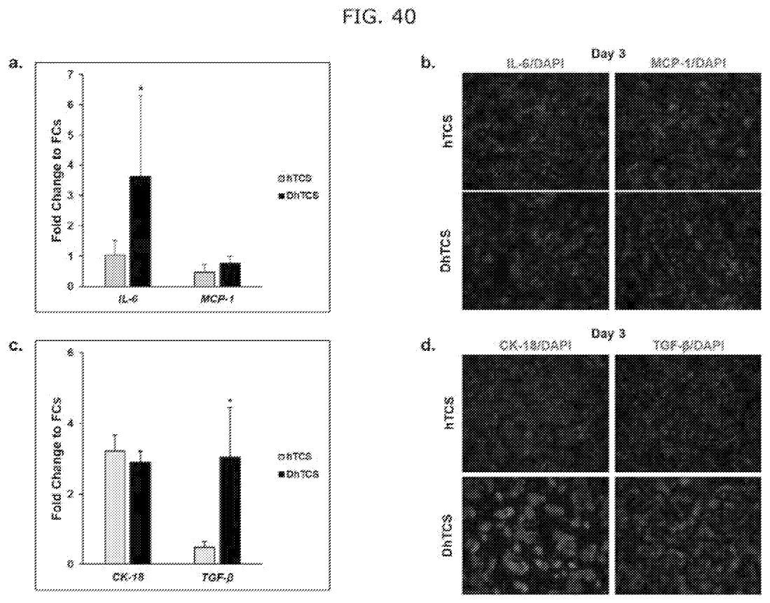

Gene and protein expression of the pro-inflammatory cytokines IL-6 and MCP-1 were determined on day 15. IL-6 gene expression was significantly higher in the DhTCS compared to the hTCS (FIG. 40a). There was ≥3-fold-change in IL-6 gene expression in the DhTCS compared to the hTCS relative to FCs resulting in a significant difference between the two systems (3.6±2.7 vs 1.0±0.5). No significant difference was seen in MCP-1 gene expression between the two systems. The fold-change for MCP-1 expression was under 2-fold for DhTCS compared to the hTCS relative to FCs (0.7±0.2 vs 0.5±0.3).

When IL-6 and MCP-1 levels were measured, the DhTCS secreted significantly higher levels of IL-6 (68.5±16.4 vs 21.1±9.7 μg/10⁶ cells/day) and MCP-1 (8,888.8±2,195.1 vs 3,482.2±1,484.5 μg/10⁶ cells/day) on day 15 (FIG. 32d). The DhTCS and hTCS were stained for expression of IL-6 and MCP-1 on days 3 (FIG. 40b) and 15 (FIG. 32e). Higher intensity staining of both were seen in the DhTCS compared to the hTCS on only day 15. After quantification of the fluorescent signal, there was a significant increase in expression in the DhTCS over the hTCS for IL-6 (41,060±12,259 vs 16,435±3,325 RFUs) and MCP-1 (33,960±9,650 vs 10,645±6,652 RFUs) on day 15 (FIG. 32f).

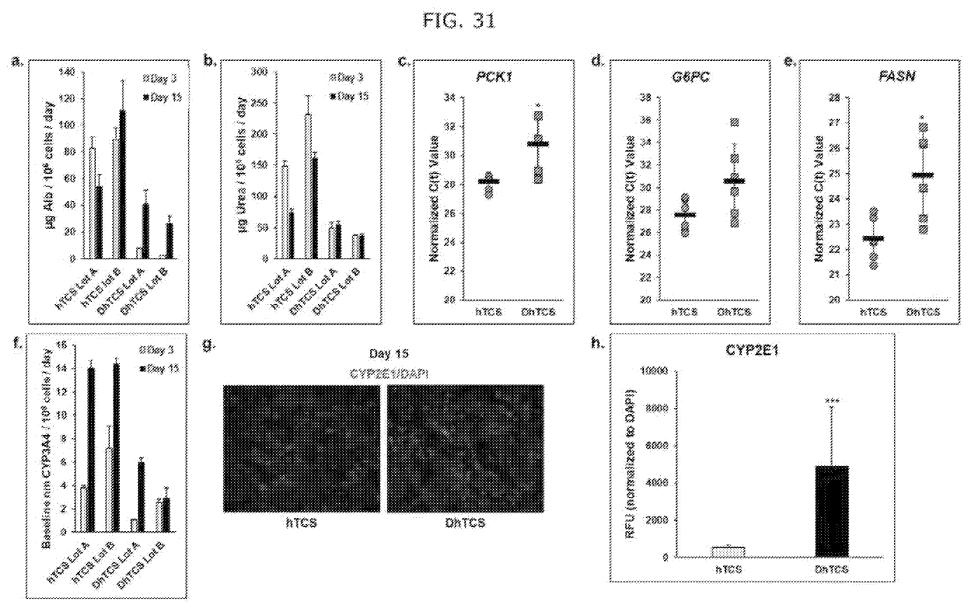

The fibrotic markers, CK-18 and TGF-β, were observed in the DhTCS and hTCS (FIG. 33). CK-18 and TGF-β gene expression was determined relative to FCs on day 15 (FIG. 40c). There was no significant difference between the DhTCS (2.9±0.3) and hTCS (3.2±0.5) for CK-18 gene expression. TGF-β gene expression was significantly higher expressed in the DhTCS (3.0±1.4) compared to the hTCS (0.5±0.2). The cells in the DhTCS secreted significantly higher levels of CK-18 (15,224±3,569 vs 9,462±2,461 pg/10⁶ cells/day) and TGF-β (753.4±322.4 vs 439.5±205.4 μg/10⁶ cells/day) compared to cells in the hTCS (FIGS. 33a-b). Higher intensity staining was seen for CK-18 and TGF-β in the DhTCS on days 3 (FIG. 40d) and 15 (FIG. 33c) compared to the hTCS. Quantification of CK-18 determined there was no change in expression between the DhTCS and hTCS from day 3 (124,629±74,838 vs 45,698±15,386 RFUs) to 15 (117,532±56,475 vs 87,264±17,596 RFUs) (FIG. 33d). TGF-β expression was significantly higher in the DhTCS compared to the hTCS on day 15 (141,968±4,079 vs 34,005±16,761 RFUs). There was a significant increase in expression in cells from the DhTCS from day 3 (29,014 #13,721 RFUs) to 15 but not in cells from the hTCS from day 3 (13,383±4,132) to 15.

### Establishing a Lipogenesis Model

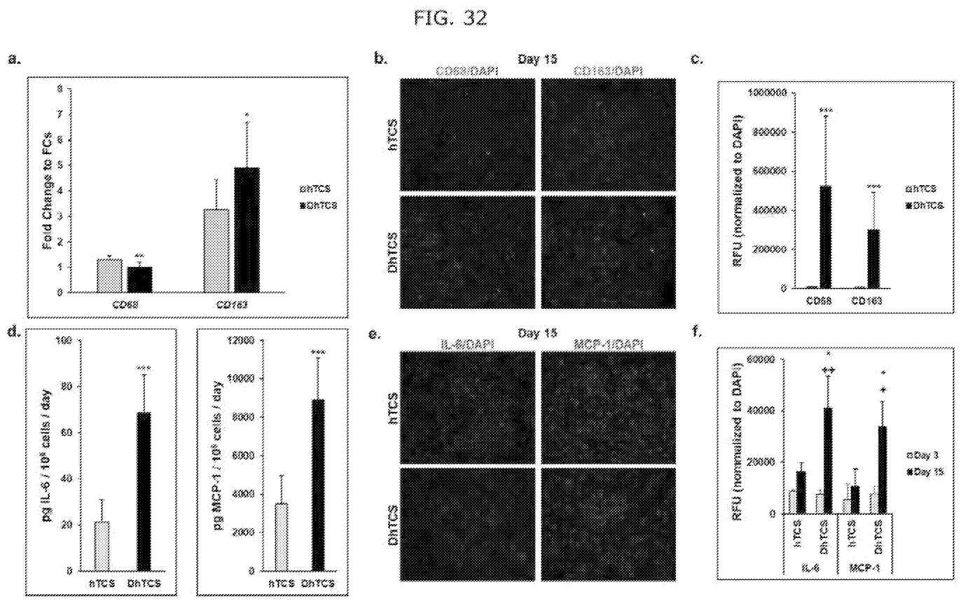

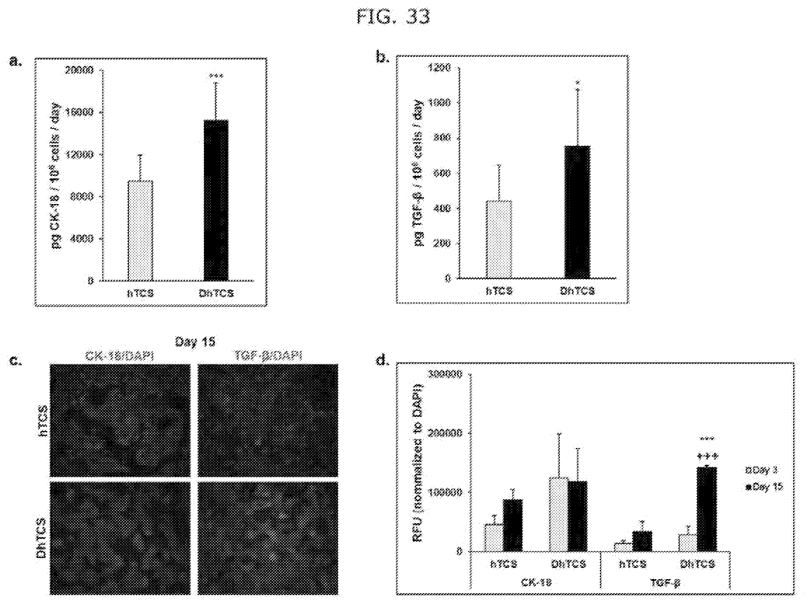

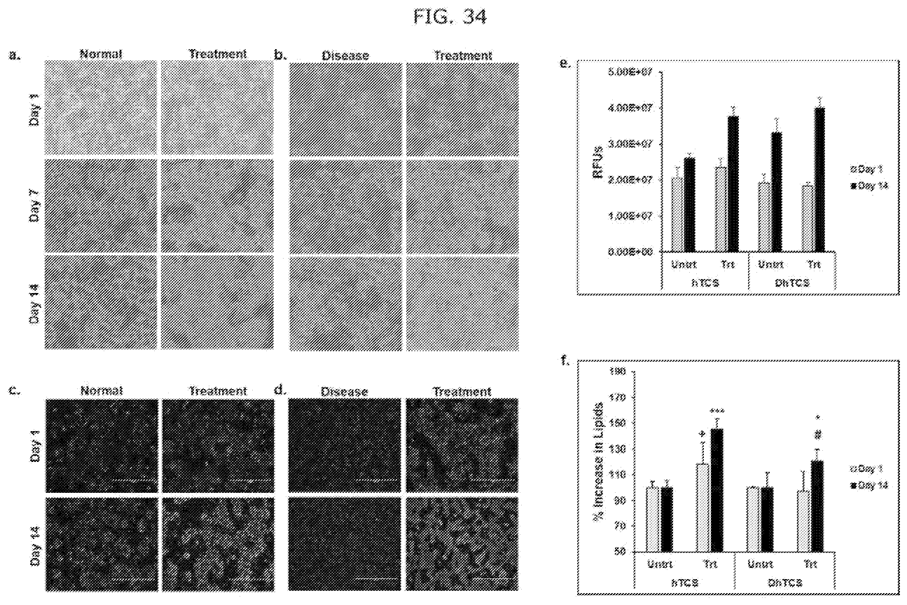

An important feature of certain liver diseases is steatosis. To increase lipid production, PHHs cultured in the hTCS and DhTCS were treated with a medium that included high glucose and the FFA oleic acid (lipogenic medium) (FIG. 34). Normal and diseased PHHs were morphologically similar in cuboidal cell shape and forming multicellular clusters (FIGS. 34a-b). Both groups of PHHs showed an increase in lipid accumulation throughout the 14-day culture period when treated with lipogenic medium with greater lipid accumulation at day 14 compared to day 1. Normal and diseased PHHs showed an increase in Nile red staining over the two-week treatment period (FIG. 34c-d). Quantification of the Nile Red staining showed similar values on day 1 across all conditions (FIG. 34e). Raw RFU values were increased in the normal PHHs in the hTCS from day 1 to 14 in the treatment group. Both untreated and treated conditions increased in the diseased PHHs in DhTCS between day 1 and 14. Normalization showed a significant increase in lipid production between untreated and treated PHHs for normal (100%±5.6 vs 145.3%±8.5) and diseased PHHs (100%±11.8 vs 120.6%±9.1) in the hTCS and DhTCS on treatment day 14 (FIG. 34f). There was a ~50% increase in Nile Red staining in treated versus untreated normal PHHs and an increase in staining of ~20% in diseased PHHs compared to treated diseased PHHs. A significant increase in lipid accumulation was seen in treated PHHs on day 1 from normal (118.2%±16.8) donors but not in diseased donors (97.2%±15.4).

Functionality of Lipogenic Medium Treated Normal and Diseased PHHs in the hTCS and DhTCS

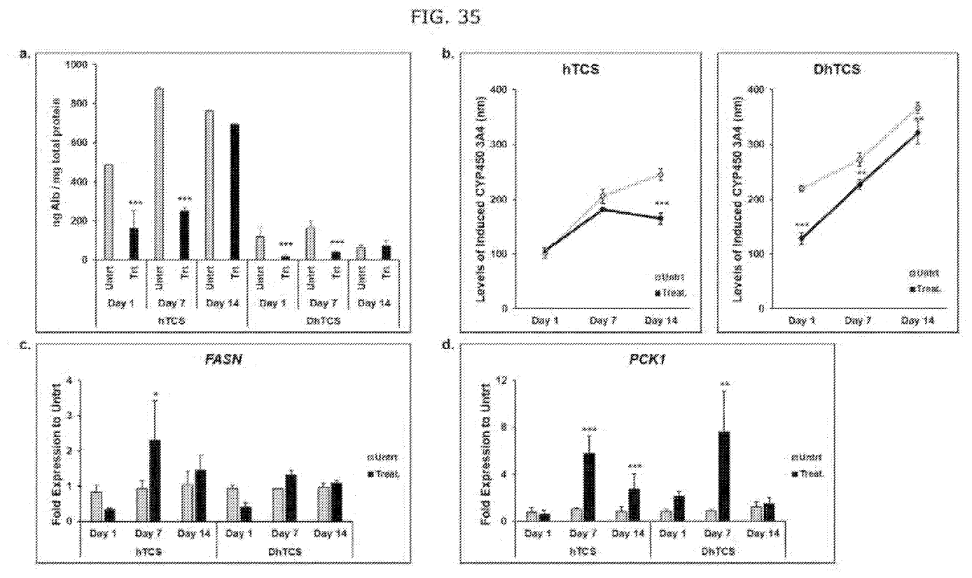

Albumin secretion and CYP3A4 activity were used as criteria to determine functionality of PHHs cultured in the hTCS and DhTCS during treatment with lipogenic medium (FIG. 35). Overall, diseased PHHs secreted less albumin compared to normal PHHs (FIG. 35a). The albumin levels decreased on days 1 and 7 in normal and diseased PHHs cultured in the treated medium. There was a rise in albumin levels over time in the treated PHHs resulting in almost equivalent levels by day 14 between both groups in the hTCS and DhTCS.

Differences in induced CYP3A4 activity were measured between untreated and treated groups in normal and diseased PHHs in the hTCS and DhTCS (FIG. 35b). No difference in induced CYP3A4 activity was seen on day 1 between untreated (101.7±10.2 nm) and treated (105.3±4.3 nm) normal PHHs. Differences in induced CYP activity were seen on treatment days 7 (181.4±0.8 nm vs 205.7±12.6 nm) and 14 (164.5±9.9 nm vs 245.5±10.5 nm) with treatment day 14 being significantly higher in the untreated samples. Lower induced CYP activity was seen in the treated compared to untreated diseased PHHs at day 1 (128.4±10.5 nm vs 219.2±5.3 nm). Treated diseased PHHs continued to have lower activity on treatment days 7 (227.3±9.2 nm vs 272.2±12.1 nm) and 14 (321.8±20.6 nm vs 367.1=10.2 nm).

Gene expression of FASN (FIG. 35c) and PCK-1 (FIG. 35d) were determined in the untreated and treated groups for normal and diseased lots. In normal and diseased PHHs, FASN gene expression was lower on treatment day 1. A significant increase in FASN gene expression was measured on treatment day 7 in normal treated PHHs (2.3±1.2) compared to untreated PHHs (0.9±0.2), but no significant increase was measured on treatment day 14 (1.5±0.4 vs 1.1±0.4). No significant differences were seen in the diseased PHHs with or without treatment on days 7 and 14. PCK-1 expression was significantly higher on treatment day 7 in the treated normal (5.8±1.5 vs 1.0±0.1) and treated diseased PHHs (7.6±3.5 vs 0.9±0.1) compared to the untreated PHHs for each group. Significantly elevated expression continued in the treated normal PHHs on day 14 (2.8±1.3 vs 0.8±0.4) but not in the treated diseased PHHs (1.5=0.6 vs 1.3±0.4).

Treatment with Lipogenic Medium Plus OCA in Normal PHHs in the hTCS

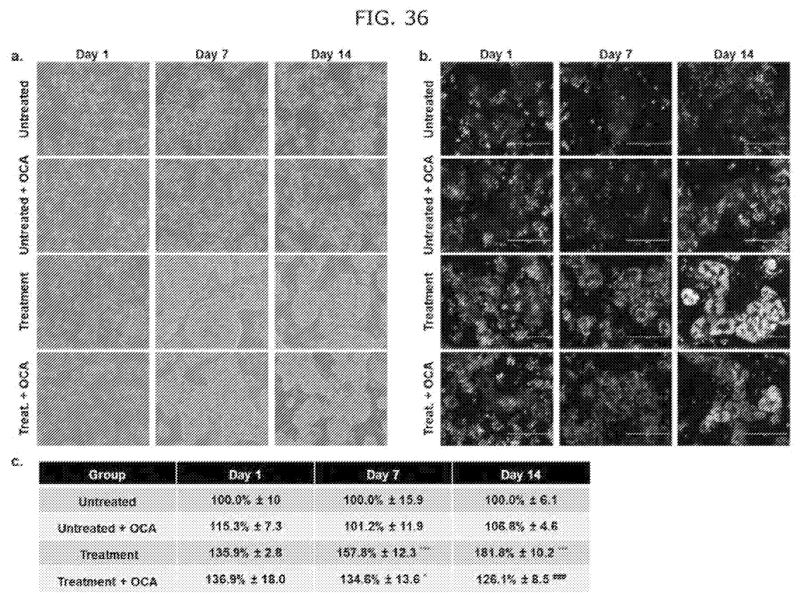

Normal PHHs were seeded in the hTCS and cultured in standard culture or lipogenic medium, with or without the addition of OCA, a potent agonist of the bile acid receptor FXR (FIG. 36). An increase in lipids was observed when PHHs were cultured in the treated medium (FIG. 36a). When OCA was added with the treated medium, less lipid accumulation was apparent on treatment days 7 and 14. Nile red staining was performed to determine lipid accumulation (FIG. 36b). There was an increase in Nile Red staining on treatment days 1 (135.9%±2.8), 7 (157.8%±12.3), and 14 (181.8%±10.2) in the treated PHHs (FIG. 36c). A decrease in lipid accumulation was seen on days 7 (134.6%±13.6) and 14 (126.1%±8.5) in the treated plus OCA PHHs compared to the treated only PHHs with day 14 having significantly less lipid accumulation.

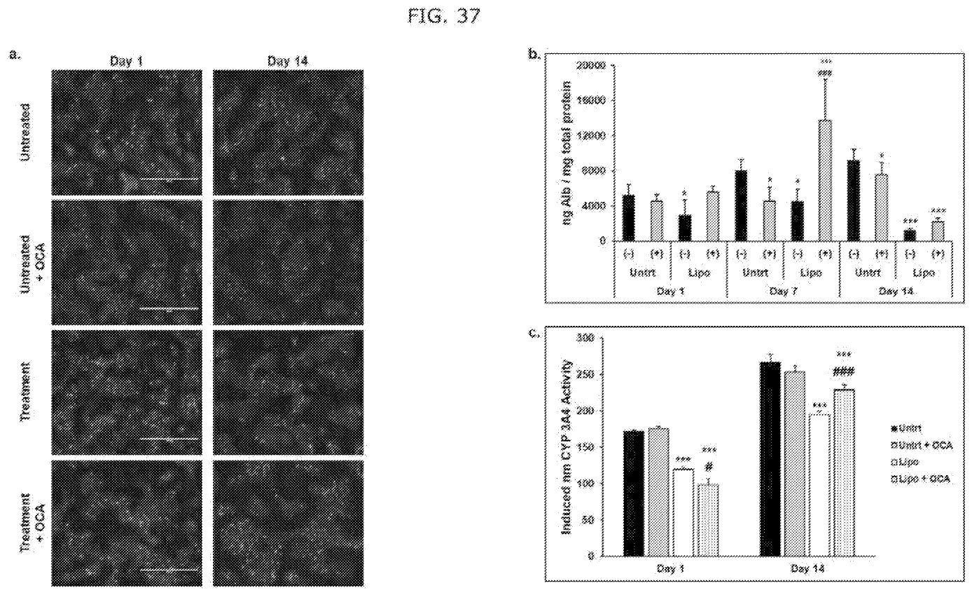

Because OCA is used to treat primary biliary cholangitis, its effect on the structure and function bile canaliculi in PHHs cultured in the hTCS was evaluated (FIG. 37). CDFDA staining showed extensive bile canaliculi formation in normal PHHs in the presence or absence of OCA on days 1 and 14 (FIG. 37a). Normal PHHs cultured in the treated medium with or without the addition of OCA appeared to increase bile canalicular network formation compared to untreated PHHs.

Albumin secretion (FIG. 37b) and induced CYP3A4 activity (FIG. 37c) were measured after treatment with or without the addition of OCA. An increase in albumin was seen in the treated plus OCA PHHs compared to the treated only group on day 1, although it was not significant. The greatest effect of OCA was measured on day 7 when the treated plus OCA PHHs had significantly higher albumin secretion (13,706±4,738 ng/mg total protein) compared to treatment only (4,527±1,393 ng/mg total protein) and untreated (7,955±1,356 ng/mg total protein). By day 14, significantly lower albumin levels overall were observed in the treatment plus OCA PHHs (2,219±400 ng/mg total protein) compared to untreated PHHs (9,142±1, 324 ng/mg total protein), resulting in only slightly higher albumin levels compared to treatment only (1,178±252 ng/mg total protein).

When induced CYP3A4 activity was measured on day 1, treated plus OCA PHHs had the lowest induced CYP activity (97.5±9.2 nm) compared to untreated (171.7±2.1 nm) and treated only (119.6±3.5 nm). On day 14, treated plus OCA PHHs had significantly higher induced CYP3A4 activity (228.8±6.7 nm) compared to treated only (195.3±4.4 nm) but lower induced activity compared to untreated PHHs (266.9±11.3 nm). Overall, CYP3A4-induced activity was improved by OCA in the presence of increased FFA and high glucose.

There is a lack of suitable in vitro model systems for liver disease studies. The hTCS was characterized as a model that uses only human cells and has stable morphology and function over 21 days of culture when compared to traditional sandwich cultures. A problem with the development of liver disease models is the inability of diseased PHHs to attach and culture for extended periods of time. Diseased PHHs were tested in the hTCS model system to determine the feasibility and characteristics in the DhTCS. Although there was a significant difference in attachment efficiency between diseased versus normal PHHs, diseased PHHs in the DhTCS were able to plate, culture, and function for at least two weeks. Diseased PHHs showed characteristics of a diseased phenotype, including significant lipid accumulation. In terms of basic liver functions, the diseased DhTCS secreted less albumin and urea, and had lower baseline CYP3A4 activity when compared to normal PHHs after normalization. Ultimately, the diseased PHHs appeared to retain some of the expected characteristics of a diseased phenotype in the DhTCS.

The decrease in albumin synthesis in the DhTCS is congruent with clinical observations of progressively impaired albumin levels in MAFLD patients. Interestingly, the diseased PHHs exhibited several more advanced features of progressing MAFLD, including inflammation. M2 macrophages were more prevalent in the DhTCS compared to the hTCS as detected by gene expression levels (CD163) and immunolocalization (CD163 and CD68) over the 14-day culture period. The chemokine MCP-1 and the pro-inflammatory cytokine IL-6 were present and had higher protein expression in the DhTCS versus the hTCS. MCP-1 is a known recruiter of macrophages and has been shown to play a role in liver disease progression. Serum levels of patients with MASH contained higher levels of IL-6 compared to NAFL patients. The presence of macrophages and elevated levels of pro-inflammatory cytokines in the DhTCS suggests that the model progressed beyond simple steatosis over the culture period.

Progression of MAFLD and other chronic liver diseases typically involves a series of inflammatory responses that lead ultimately to the initiation of fibrogenesis and the progressive accumulation of insoluble collagen fibrils that impede intercellular communication resulting in end-stage disease. Increased expression of both CK-18 and TGF-β suggest that fibrogenic signaling cascades were activated in the DhTCS. The fibrosis scores from the donors were all ≤1; therefore, the severity of fibrosis would be expected to be minimal without further induction. Future studies will further characterize the latter stages of the model and quantify the relative contribution of collagen I and collagen IV to the fibrogenic response in the DhTCS.

Gene expression related to fat and sugar utilization was also altered in the diseased versus normal PHHs. In the DhTCS, expression of the FASN gene was lower in diseased PHHs compared to normal PHHs in the hTCS. Human livers have significantly higher expression of FASN in steatosis without significant inflammation as compared to control and MASH livers. It may be that FASN expression is determined by the severity of disease. The diseased PHHs being tested in the DhTCS could be in a “transitory phase” where steatosis and inflammation are present. Disease severity is progressing to a fibrogenic state, resulting in a more advanced phenotype, and a potential decrease in gene expression. A similar gene expression pattern was seen for PCK1 when transcriptome data sets were compared. As severity of the disease increased from obesity to MAFLD to MASH, PCK1 expression decreased. Gene expression from diseased PHHs cultured in the DhTCS was decreased for both FASN and PCK1 genes compared to normal PHHs cultured in the hTCS, suggesting a moderately advanced diseased state. Further studies will assess whether glucose production is affected in both diseased and normal PHHs cultured in the DhTCS and hTCS.

The DhTCS can support an “early stage” disease model through the addition of disease-relevant stimuli. A diseased state can be induced in both the hTCS and DhTCS by exposing the system to a combination of glucose and FFAs such as oleic acid. These two major stimuli, designated as lipogenic, were used to perturb normal and diseased PHHs. Differences in functionality between the treated diseased and normal PHHs were seen in albumin secretion and induced CYP3A4 activity. FFA or high glucose treatment have shown similar decreases in CYP3A4 activity. These results agree with the decreased activity that was measured in the more advanced diseased PHHs without treatment in the DhTCS. Both types of PHHs showed a significant increase in lipid accumulation throughout the two-week culture period, although this increase was less pronounced in the treated diseased versus normal PHHs. The diseased PHHs may inherently have lower hepatic function and activity and higher lipid accumulation to begin with, therefore additional lipogenic stimuli will have less effect compared to normal PHHs. Overall, diseased PHHs in the DhTCS seemed to be less responsive to the lipogenic treatment compared to normal PHHs in the hTCS.

The increased sensitivity to lipogenic treatment of diseased PHHs was seen in gene expression. PCK1 expression was increased in the treated normal and diseased PHHs on day 7. Because PCK1 plays a critical role in gluconeogenesis, the increase in gene expression suggests that changes in glucose production and insulin sensitivity may have occurred in the normal and diseased PHHs when treated with lipogenic media. However, treated diseased PHHs appear less sensitive to this treatment perhaps due to their current state of disease. A similar result was seen in FASN gene expression with only treated normal PHHs showing increased expression. Normal PHHs cultured in a micropatterned co-culture exposed to hyperglycemic conditions for 3 weeks had increased FASN and PCK1 gene expression on day 10 but then decreased on day 18, similar to the expression pattern seen in the treated normal PHHs in the hTCS. Based on these differences in response to treatment, normal PHHs cultured with lipogenic stimuli may be useful for “early stage” diseased modeling where steatosis is important while the use of diseased PHHs cultured in the DhTCS without stimuli may be applicable to a later stage of disease modeling.

Notably, the differences observed in attachment efficiency, gene expression profiles and other functional endpoints between hepatocytes isolated from donor tissues with lower versus higher NAS scores could be due several cellular, positional, and pathological factors. It is not clear how much the attached cell populations are a representation of the overall hepatocyte populations from the respective tissues or how much they may represent a subpopulation that are being selected for during the thawing and seeding process. Marked differences in many facets of hepatocyte maturation, gene expression and disease biomarkers have been noted across the microarchitecture of the liver, e.g. periportal vs pericentral regions. The cellular and molecular basis of these phenotypic differences observed in the hTCS have yet to be explored and may warrant further investigation.

An important highlight of the hTCS is its potential to be used for drug treatment studies. FXR is a nuclear transcription factor activated by bile acids and influences bile acid synthesis, transport, and reabsorption. Upon bile acid binding to the FXR, circulating triglycerides and gluconeogenesis from hepatocytes has been shown to decrease. The FXR agonist OCA has been tested in clinical trials for treatment of liver disease including steatosis, inflammation, and fibrosis. It has been shown to have effects on glucose metabolism and insulin sensitivity and remains a promising candidate for treatment of MASH. Upon lipogenic treatment, OCA had a positive effect of decreasing lipogenesis in treated normal PHHs, a similar result found previously, and affected the formation of the network of bile canaliculi. Since OCA is used to treat primary biliary cholangitist, a cholestatic disorder, formation of a more extensive bile canaliculi suggests the choleretic effects of OCA can be detected in the hTCS with treated normal PHHs. Hepatic functions, including albumin levels and CYP3A4 activity, were improved or restored in the PHHs cultured in lipogenic media upon OCA treatment. Treatment of PHHs with FFA has been shown to decrease CYP3A4 activity; in contrast, an increase in CYP activity was seen when OCA was added. Overall, OCA reduced lipid accumulation and improved albumin levels and CYP3A4 activity in the hTCS, demonstrating the potential suitability of the system for evaluating drug therapies. Future studies include treating diseased PHHs with OCA in the DhTCS and evaluating OCA's effect on hepatocyte function and steatosis as well as mechanisms of bile acid mobilization and transport.

Diseased PHHs cultured in the DhTCS were able to sustain distinct features of fatty liver disease, including decreased hepatocyte functionality and increased lipogenesis. The DhTCS showed pro-inflammatory cytokine production and elements of fibrogenesis. Diseased and normal PHHs responded to the lipogenic stimuli of high glucose and FFA by showing disease-associated features. Treatment with OCA, a drug known to affect hepatic steatosis, modulated lipogenesis and functionality in lipogenic conditions. The DhTCS is a flexible and easy-to-use platform for investigation of liver disease at either an early “initiating” stage or a later “progressing” stage. Modulation of the standard hTCS model can be achieved by using disease-origin PHH (DhTCS) or introducing perturbations such as exposure to inducers of pathogenesis. Moreover, the longevity of the DhTCS system creates the potential for interventional strategies to be applied across the spectrum from prevention to treatment of advanced disease features.

### Materials and Methods

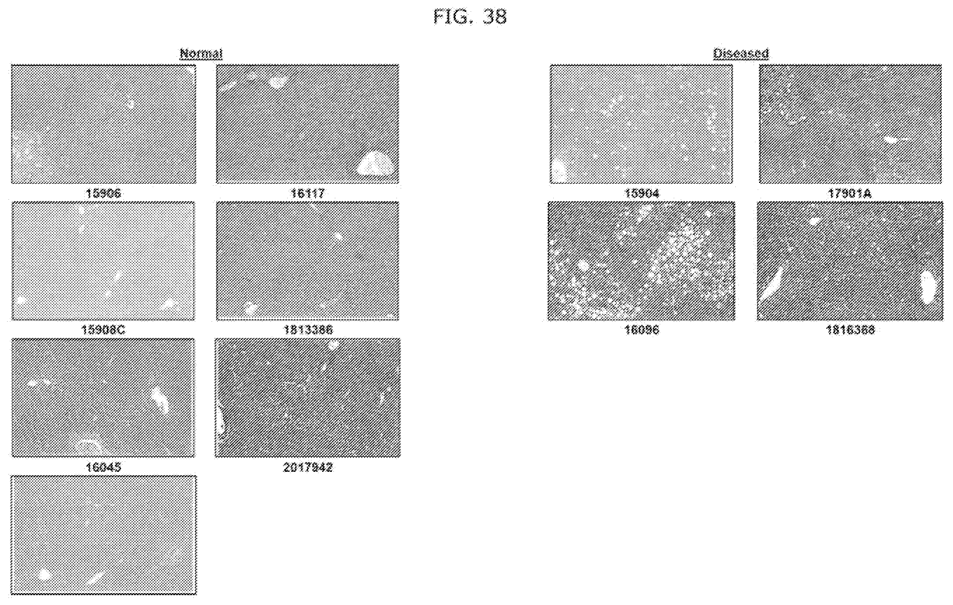

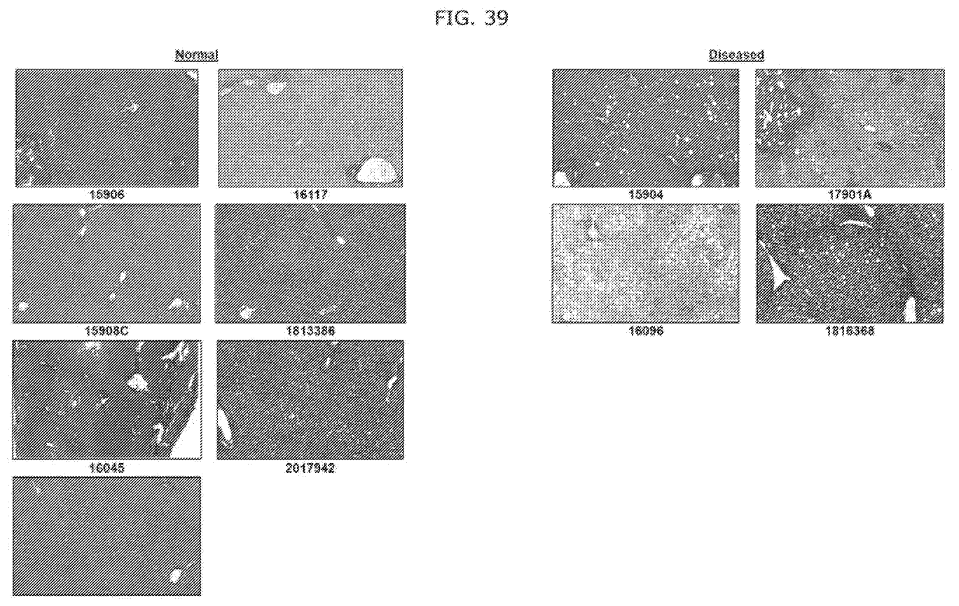

All-Human Triculture System. The hTCS was set up as previously described, including the medias, cell types, and expected functional performance. This protocol follows the guidelines of LifeNet Health's ethics committee. All cells were isolated from donor tissue fully consented for research purposes by LifeNet Health. PHHs have been designated as normal or diseased based on a histopathologic assessment of the tissue of origin by a board-certified pathologist, using the standard MASH CRN Scoring System. Formalin-fixed paraffin-embedded tissue sections were stained with H&E (FIG. 38) and Masson's Trichrome (FIG. 39) and assessed for steatosis, lobular inflammation, and hepatocyte ballooning to form the MAFLD Activity Score (NAS). Fibrosis was scored separately on a 4-point scale according to the classification as described by Kleiner et al (Kleiner et al., 2005). Tissues with a NAS of ≥4 were designated “Diseased” (n=4) while those with NAS of ≤3 were categorized as “Normal” (n=7) (Table 1).

Morphological Assessment, PHH Attachment, and Percent Confluency. To morphologically assess the cells, they were imaged on the designated days using a BX41 microscope with a 10× objective (Olympus, Tokyo, Japan). The previously described method was used to determine PHH attachment and percent confluency.

Albumin and Urea Assays. Supernatant was collected on the indicated days for measurement of albumin and urea. Three wells or greater were designated for each condition. Samples were run in duplicate. The concentration of albumin was determined using an ELISA assay (Abcam, Cambridge, MA) and performed according to the manufacturer's instructions. Urea was measured by a colorimetric kit (Stanbio, Boerne, TX) and performed according to the manufacturer's instructions.

Bicinchoninic Acid Assay (BCA). The BCA assay was performed with supernatant collected on the designated day. Three wells or greater were designated for each condition. Samples were run in duplicate. Total protein concentration was determined using a kit (Thermo Fisher, Waltham, MA) and performed according to the manufacturer's instructions.

CYP3A4 assay. To induce CYP3A4 enzyme, cells were treated with 25 μM Rifampicin (Sigma) in supplemented culture medium at 37° C. for 48 hours prior to measuring CYP3A4 enzyme activity on the specified day. Three wells or greater were designated for each condition. Samples were run in duplicate. The P450-Glo Assay kit was used to detect CYP3A4 enzyme activity (Promega, Madison, WI). After 48 hours, the medium was removed, and the cells were washed with DMEM (no phenol red) (Thermo Fisher). Cyp-Luciferin-IPA stock was then added and incubated at 37° C. for 30 minutes. The supernatant was then collected, and the assay was performed according to the manufacturer's instructions.

Gene expression. Cells were lysed using RLT buffer (Qiagen, Germantown, MD). RNA was then isolated using the RNeasy kit (Qiagen) as per the manufacturer's instructions. cDNA was prepared using the PrimeScript RT reagent kit (Takara Bio, Shiga, Japan) in a 30 μL volume reaction containing 5× PrimeScript RT Master Mix, RNA, and ddH₂O. qRT-PCR reactions contained 10 μL QuantiNova SYBR Green Master Mix (Qiagen), 2 μL ROX reference dye (Qiagen), 2 μL designated primer set (10 μM), 1 μL cDNA, and 5 μL ddH₂O for a final volume of 20 μL. Primers used for Glyceraldehyde 3-Phosphate Dehydrogenase (GAPDH), Fatty Acid Synthase (FASN), Phosphoenolpyruvate Carboxykinase 1 (PCK-1), and Glucose-6-Phosphatase Catalytic Subunit (G6PC) are listed in Table 2 (Thermo Fisher). Taqman primers were used for CD68 and CD163 (Thermo Fisher). PCR amplification was done on a StepOne Plus Real Time PCR System (Applied Biosystems) and analyzed with StepOne software (version 2.3) (Applied Biosystems) and Microsoft Excel. Gene expression was normalized to the housekeeping gene GAPDH and analyzed using the 2^{-ΔΔC}_{T} method. Data is presented as fold change relative to Feeder Cell (FC) expression or Untreated PHHs.

Immunofluorescence. Cells were fixed with a Fixation Solution (eBioscience, San Diego, CA) for 30 mins at room temperature. They were then washed two times with 1× Permeabilization (eBioscience), and primary antibody was added at 4° C. overnight. The following antibodies were used: 1) CYP2E1 at 1:100 (Abcam, ab28146); 2) CD68 at 1:100 (Abcam, ab213363); 3) CD163 at 1:100 (Abcam, ab87099); 4) anti-Monocyte Chemoattractant Protein-1 (MCP-1) at 1:100 (Abcam, ab9669); 5) anti-Interleukin-6 (IL-6) at 1:100 (Abcam, ab233706); 6) anti-CK-18 at 1:100 (Abcam, ab24561); and 7) anti-TGF-β at 1:100 (Abcam, ab92486). Cells were washed twice, and secondary goat anti-mouse IgG Alexa Fluor 488 conjugated antibody (Thermo Fisher) or secondary goat anti-rabbit IgG Alexa Fluor 555 conjugated antibody (Thermo Fisher) were added at a 1:500 dilution for 30 minutes (mins) at 4° C. Cells were then washed twice, and Fluoromount-G mounting medium with DAPI (Invitrogen, Waltham, MA) was added for 20 mins at room temperature. Images were taken using a Zeiss Observer.Z1 fluorescent microscope (Zeiss, Dublin, CA). Images were acquired with Z-stack using a Zeiss ApoTome.2 on the 20× objective lens. Gray values of CYP2E1, CD68, CD163, MCP-1, IL-6, CK-18, and TGF-β were normalized to gray values of DAPI using Image J software (https://imagej.nih.gov/ij/).

Glucose, Oleic Acid, and OCA Treatment. In the indicated experiments, cells were treated daily with lipogenic medium which included 25 mM glucose (Sigma) and 320 μM Oleic acid (Sigma) beginning on day 3 of the culture period and ending on day 17. The first day of treatment is “Day 1”, and the last day of treatment is “Day 14”. When indicated, 6α-ethyl-chenodeoxycholic acid (OCA) (AdipoGen Life Sciences, San Diego, CA) was added daily to the cells at a final concentration of 0.5 μM throughout the Lipogenic treatment period.

Nile Red Staining. Cells were washed with 1×DPBS (—Ca⁺⁺/—Mg⁺⁺) (Thermo Fisher) three times, and then Nile Red was added at a 1:500 dilution (Abcam). After fifteen mins at 37° C., cells were washed twice with 1×DPBS (—Ca⁺⁺/—Mg⁺⁺) and then imaged on an EVOS FL cell imaging system (Thermo Fisher) using a 10× objective. Nile Red staining was quantified by determining the densitometric fluorescence value (red channel) using Image J.

5-(and-6)-Carboxy-2′, 7′-Dichlorofluorescein Diacetate Staining. To visualize bile canaliculi formation, cells were rinsed twice with 1×DPBS (—Ca⁺⁺/—Mg⁺⁺) (Thermo Fisher). Fresh medium plus 5 μM 5-(and-6)-Carboxy-2′, 7′-Dichlorofluorescein Diacetate (CDFDA) (Thermo Fisher) were added to the cells and incubated for 20 minutes at 37° C. Cells were then washed twice with 1×DPBS (—Ca⁺⁺/—Mg⁺⁺), and DMEM (no phenol red) (Gibco) was added to the cells. Images were taken with an EVOS FL cell imaging system (Thermo Fisher) using a 10× objective.

Calculating PHH Attachment and Statistical Analysis. Images shown are from representative donor lots. All experiments were performed using a minimum of 3 technical replicates for each collection point. Values were normalized to the determined number of attached PHHs as described above except when noted otherwise. Significance was calculated in MiniTab (State College, PA) using one-way ANOVA with Tukey post hoc testing to determine statistical significance with 95% confidence and *p<0.05 for statistical significance.

TABLE 1
Donor Characteristics. Donor characteristics include age, sex, race, and BMI.
Non-Alcoholic Fatty liver Disease (NAS) score, steatosis score, lobular inflammation
score, hepatocyte ballooning score, and fibrosis stage are also listed.

| | | | | | | | Lobular | Hepatocyte | |
|---|---|---|---|---|---|---|---|---|---|
| | | | | | NAS | Steatosis | Inflammation | Ballooning | Fibrosis |
| Donor | Age | Sex | Race | BMI | Score | Score | Score | Score | Stage |
| 15906 | 28 | F | Caucasian | 26 | 0 | 0 | 0 | 0 | 0 |
| 15908C | 27 | F | A/A | 31 | 1 | 1 | 0 | 0 | 0 |
| 16045 | 23 | M | Caucasian | 34 | 0 | 0 | 0 | 0 | 0 |
| 16117 | 41 | F | Caucasian | 30 | 0 | 0 | 0 | 0 | 0 |
| 1813386 | 42 | M | A/A | 31 | 0 | 0 | 0 | 0 | 0 |
| 15102 | 31 | F | Caucasian | 37 | 3 | 2 | 0 | 0 | 0 |
| 2017942 | 55 | F | Caucasian | 24 | 0 | 0 | 0 | 0 | 0 |
| 17901A | 50 | F | Caucasian | 43 | 5 | 2 | 1 | 2 | 0 |
| 15904 | 54 | M | Caucasian | 36 | 5 | 3 | 1 | 1 | 1 |
| 16096 | 73 | F | Caucasian | 30 | 4 | 2 | 1 | 1 | 0 |
| 1816368 | 32 | M | Caucasian | 43 | 6 | 3 | 1 | 2 | 0 |

TABLE 2
Primer sequences for RT-PCR. Primer sequences are
listed for Glyceraldehyde-3-phosphate dehydroge-
nase (GAPDH), Fatty Acid Synthase (FASN),
Phosphoenolpyruvate Carboxykinase (PCK-1), and
Glucose-6-Phosphate Catalytic subunit (G6PC)
genes.

| | |
|---|---|
| GAPDH | Forward: 5′-GGTCACCAGGGCTGCTTTTA-3′ |
| | Reverse: 5′-GGATCTCGCTCCTGGAAGATG-3′ |
| FASN | Forward: 5′-AAGGACCTGTCTAGGTTTGATGC-3′ |
| | Reverse: 5′-TGGCTTCATAGGTGACTTCCA-3′ |
| PCK-1 | Forward: 5′-ACTCGAGGTTCTGCACCCCT-3′ |
| | Reverse: 5′-AGGCAGCATCAATGATGGG-3′ |
| G6PC | Forward: 5′-TCATCTTGGTGTCCGTGATCG-3′ |
| | Reverse: 5′-TTTATCAGGGGCACGGAAGTG-3′ |

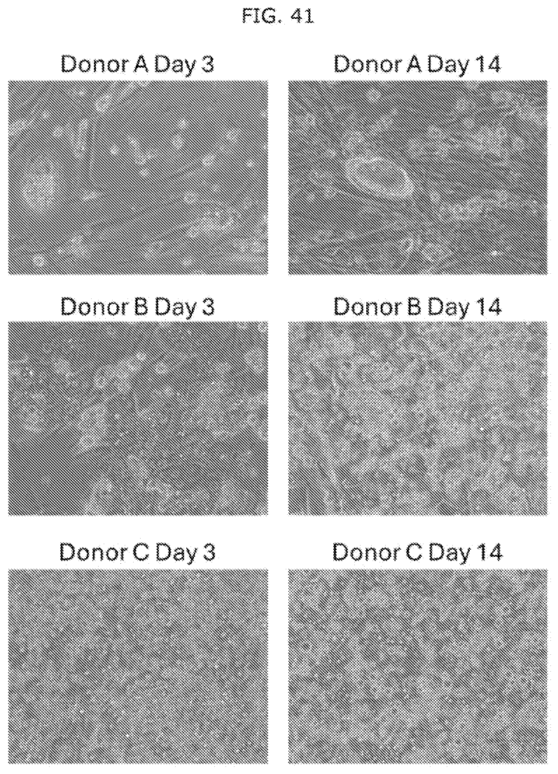

In general, diseased PHHs in the DhTCS retained stable phenotype and function for extended culture periods resulting in reduced hepatic functions of albumin synthesis, urea production, and CYP3A4 activity when compared to healthy-origin PHHs. Examples of diseased PHHs cultured in the DhTCS are shown in FIG. 41. Disease relevant features included lipid accumulation, CYP2E1 upregulation, and presence of Cytokeratin 18 (CK-18) and TGF-β in the DhTCS. Induction of steatotic change and decreased hepatic functionality occurred upon exposure to lipogenic media and was modulated by OCA. The presence and maintenance of these diseased characteristics suggests the potential of the DhTCS to be a suitable platform for development of liver disease models.

### Example 9

The purpose of this study was to develop an in vitro strategy using an all-human 2D+ hepatic system, with Kupffer cells (KCs) to assess initial fibrogenic responses following treatment with a compound.

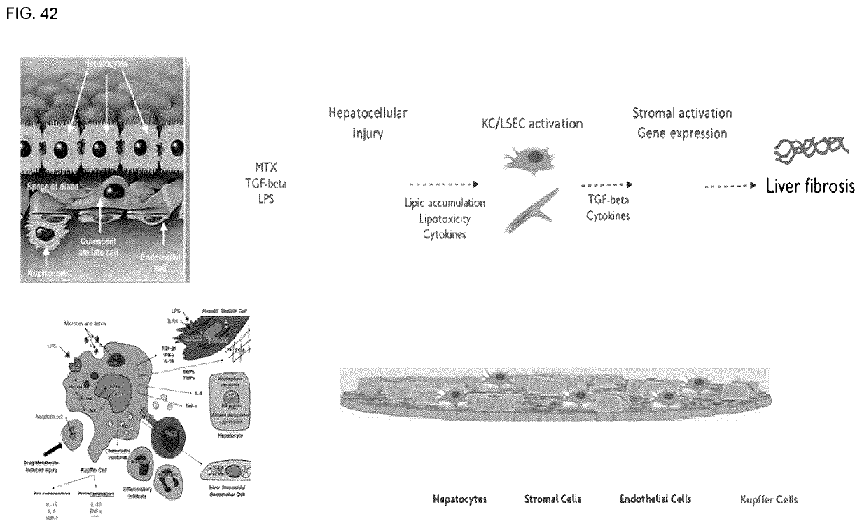

Methods: The hepatic tri-culture system, including adult primary human hepatocytes (PHHs), endothelial cells and stromal cells, was cultured with and without primary KCs, at a ratio of 1 KC:4 PHH or 1 KC:2 PHH, in a glucocorticoid-containing or -free media. FIG. 42 shows schematics of the cells and the steps leading to liver fibrosis. MTX or TGF-β1 or liposaccharides (LPS) cause hepatic injury. The injury leads to lipid accumulation, lipotoxicity and cytokines are produced. These activate the KCs and the liver sinusoidal endothelial cells (LSEC). Then stromal activation and gene expressions occur, leading to liver fibrosis.

Following exposure to methotrexate (MTX; 0.1 and 1 μM; Sigma-Aldrich, St. Louis, MO) or TGF-β1 (0.1 and 10 ng/ml; Miltenyi Biotec, Gaithersburg, MD) or vehicle (DMSO; 0.1% v/v), the expression of various genes known to have a role in fibrogenic responses (e.g., LGALS3, LOX, MMP1) was determined. MTX and TGF-β1 are known fibrogenic compounds Cell health was assessed by urea production and LDH release in the medium.

The experiments were done in a 96-well format according to the details shown below. FIG. 43 shows the plate layouts for the experiments. The hepatocytes were from an individual adult lot. The Kupffer cells (KCs) were from an individual adult lot. The seeding density of the Kupffer cells to the hepatocytes was selected to mimic healthy or inflammatory physiological levels (1:4 or 1:2 ratio). The seeding of the KCs occurred on Day 1 of the culture (meaning 24 hours post-hepatocyte plating).

### Treatment Details:

Modifications to the medium: Feeder Cell Thawing Medium (FCTM)—Used as is; human hepatocyte thawing medium (HHTM)—Used as is; Supplement A—No hydrocortisone present; Supplement B—Used as is; Supplement C—Used as is. These are the Supplements A, B and C from LifeNet Health.

TruVivo plating medium (TCPM) (1 μM Dexamethasone “Dex”)—Modifications made: TruVivo plating medium (TCPM) base medium (as is); Supplement A (as is); Supplement B (as is). All are available from LifeNet Health.

TruVivo Culture Medium (TCCM) for conditions with Dex (0.1 μM Dex)—Modifications made: TCCM base medium (as is); Modified Supplement A (no hydrocortisone); Supplement C (as is).

TCCM for No Dex conditions—Modifications made: TCCM base medium (as is); Modified Supplement A (no hydrocortisone); Insulin-Transferrin-Selenium [ITS+: Corning ITS+ Premix: As an aqueous solution containing human recombinant insulin, human transferrin (12.5 mg each), selenous acid (12.5 μg), bovine serum albumin (BSA) (2.5 g), and linoleic acid (10.7 mg)] used (used in place of Supplement C).

All hepatocyte plating was performed using TCPM with Dex (1 μM), as above. Four hours after hepatocyte plating, TCCM was added with and without Dex (half of the cultures with Dex, half without.) The media used were those as described above. The medium exchange process was to do 50% media changes following KC plating up until the compound addition.

Fibrogenic reference compounds: Positive Controls were TGF-β1 (0.1 and 10 ng/ml) and Methotrexate (0.1 and 1.0 μM).

The timeline and workflow was as follows.

Thawing and plating of the hepatocytes was done according to methods as known in the art. The next day (24 hours) after plating the cells, the KCs were added in TCCM with or without Dex (0.1 μM) at a 1:4 or 1:2 ratio to hepatocytes. The next day and subsequent days, 50% volume of medium was changed each day. Starting at day 5 of culture, the compounds tested were added in the 50% medium volume and allowed to go, unchanged, through Day 8 of culture (72 hr treatment); or to 120 hr treatment times. The treatment compounds were methotrexate (0.1 and 1.0 μM) and TGF-β1 (0.1 and 10 ng/ml).

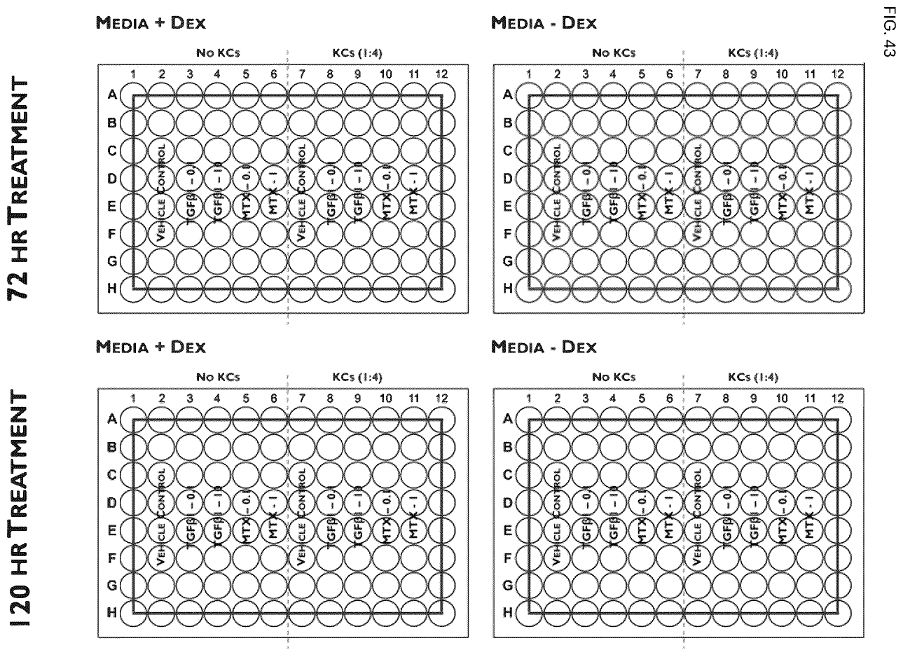

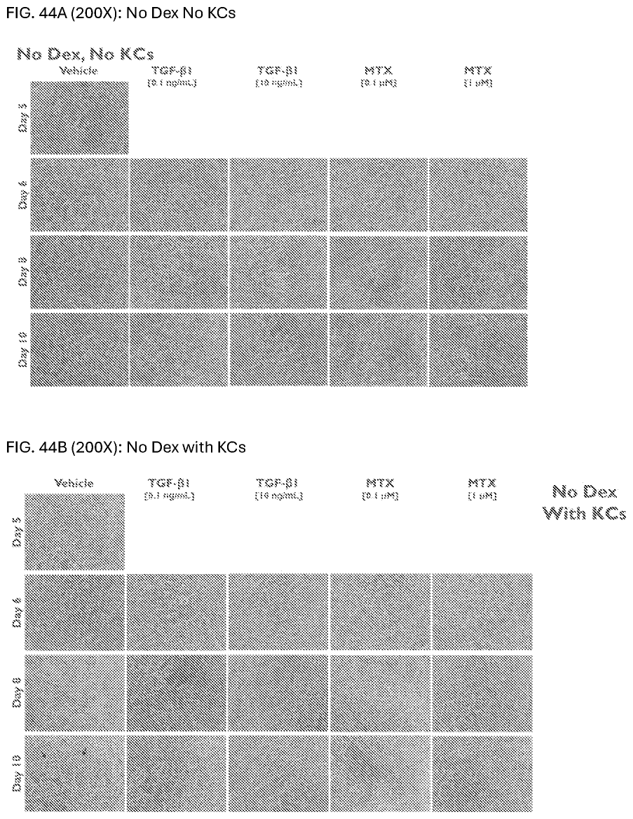

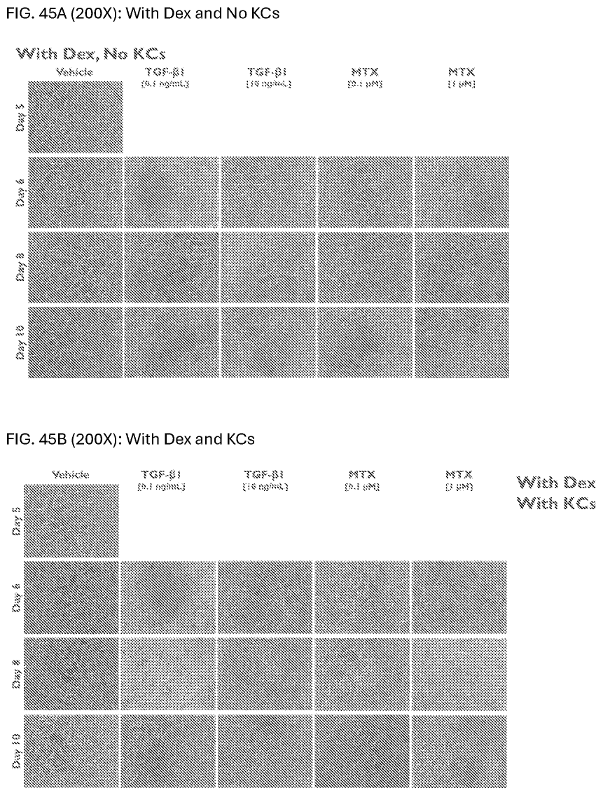

The cell harvest and analytical endpoints were done as follows. Phase contrast images were captured each day. The media samples were collected for lactate dehydrogenase (LDH) assay at the end of each treatment period (i.e., 72 or 120 hours of treatment with each compound). The results were used to assess cell health. Cell lysates were harvested for gene expression analysis. 2 wells were combined per sample. The treatment samples were analyzed in triplicate. The following genes that have a role in fibrogenic response were observed. LGALS3 (Galectin3); LOX (lysyl oxidase), MMP1 (matrix Metalloproteinase 1); Housekeeping gene (β-actin). The results are shown in FIGS. 44-49. The images in FIGS. 44 and 45 are at 200×.

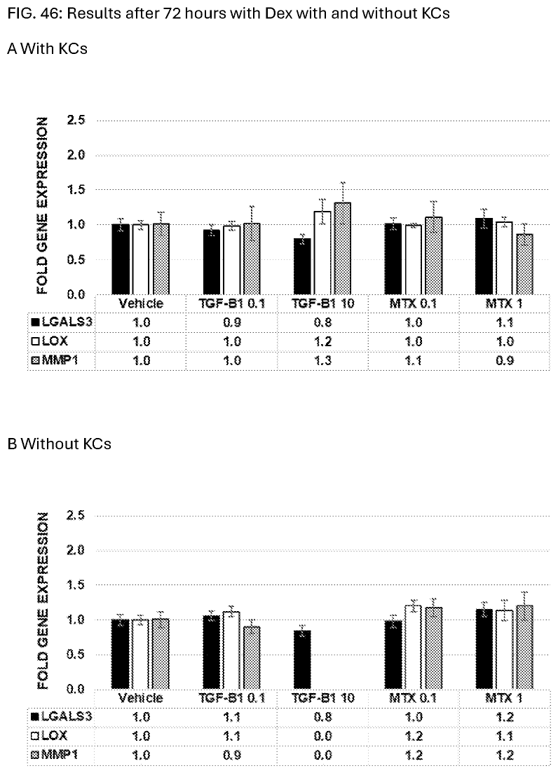

Tables 3-6 show the data plotted in FIGS. 46-49, respectively.

TABLE 3
(FIG. 46): Results after 72 hours with Dex with and without KCs
No KC

| | | | LGALS3 | | LOX | | MMP1 | |
|---|---|---|---|---|---|---|---|---|
| | | | fold | | fold | | fold | |
| | | | induction | | induction | | induction | |
| Treatment | Conc | | Mean | SD | Mean | SD | Mean | SD |
| Vehicle | 0.1%(v/v) | | 1.0 | 0.1 | 1.0 | 0.1 | 1.0 | 0.1 |
| TGF-β1 0.1 | 0.1 | ng/mL | 1.1 | 0.1 | 1.1 | 0.1 | 0.9 | 0.1 |
| TGF-β1 10 | 10 | ng/mL | 0.8 | 0.1 | * | * | * | * |
| MTX 0.1 | 0.1 | μM | 1.0 | 0.1 | 1.2 | 0.1 | 1.2 | 0.1 |
| MTX 1 | 1 | μM | 1.2 | 0.1 | 1.1 | 0.1 | 1.2 | 0.2 |
| Vehicle | 0.1%(v/v) | | 1.0 | 0.1 | 1.0 | 0.1 | 1.0 | 0.2 |
| TGF-β1 0.1 | 0.1 | ng/mL | 0.9 | 0.1 | 1.0 | 0.1 | 1.0 | 0.2 |
| TGF-β1 10 | 10 | ng/mL | 0.8 | 0.1 | 1.2 | 0.2 | 1.3 | 0.3 |
| MTX 0.1 | 0.1 | μM | 1.0 | 0.1 | 1.0 | 0.0 | 1.1 | 0.2 |
| MTX 1 | 1 | μM | 1.1 | 0.1 | 1.0 | 0.1 | 0.9 | 0.2 |

* Results that cannot be calculated are for treatment groups that could not be run with all Taqman assays due to low mRNA levels

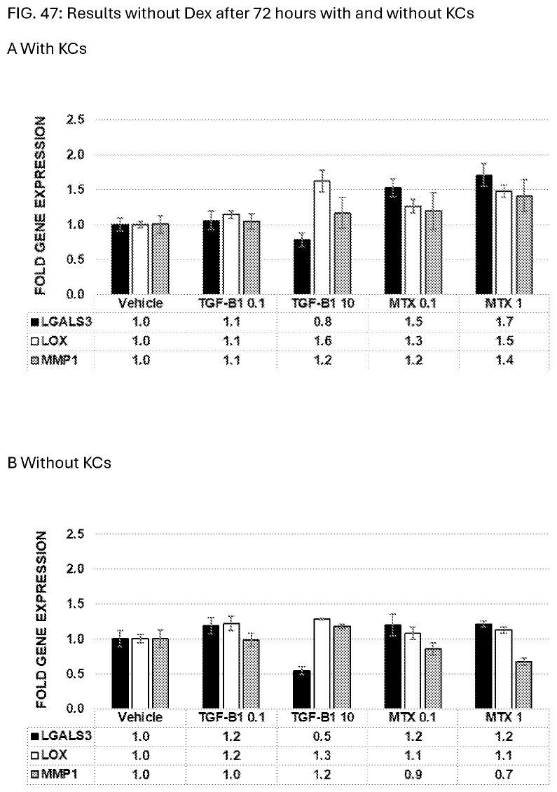

TABLE 4
(FIG. 47): Results after 72 hours without Dex with and without KCs
No KC

| | | | LGALS3 | | LOX | | MMP1 | |
|---|---|---|---|---|---|---|---|---|
| | | | fold | | fold | | fold | |
| | | | induction | | induction | | induction | |
| Treatment | Conc | | Mean | SD | Mean | SD | Mean | SD |
| Vehicle | 0.1%(v/v) | | 1.0 | 0.1 | 1.0 | 0.1 | 1.0 | 0.1 |
| TGF-β1 0.1 | 0.1 | ng/mL | 1.2 | 0.1 | 1.2 | 0.1 | 1.0 | 0.1 |
| TGF-β1 10 | 10 | ng/mL | 0.5 | 0.1 | 1.3 | 0.0 | 1.2 | 0.0 |
| MTX 0.1 | 0.1 | μM | 1.2 | 0.2 | 1.1 | 0.1 | 0.9 | 0.1 |
| MTX 1 | 1 | μM | 1.2 | 0.0 | 1.1 | 0.0 | 0.7 | 0.1 |
| Vehicle | 0.1%(v/v) | | 1.0 | 0.1 | 1.0 | 0.0 | 1.0 | 0.1 |
| TGF-β1 0.1 | 0.1 | ng/mL | 1.1 | 0.1 | 1.1 | 0.1 | 1.1 | 0.1 |
| TGF-β1 10 | 10 | ng/mL | 0.8 | 0.1 | 1.6 | 0.2 | 1.2 | 0.2 |
| MTX 0.1 | 0.1 | μM | 1.5 | 0.1 | 1.3 | 0.1 | 1.2 | 0.3 |
| MTX 1 | 1 | μM | 1.7 | 0.2 | 1.5 | 0.1 | 1.4 | 0.2 |

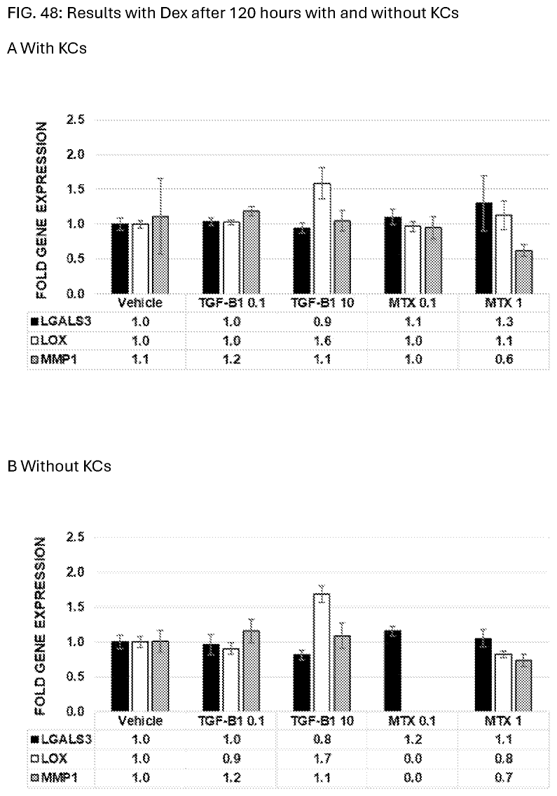

TABLE 5
(FIG. 48): Results after 120 hours with Dex with and without KCs
No KC

| | | | LGALS3 | | LOX | | MMP1 | |
|---|---|---|---|---|---|---|---|---|
| | | | fold | | fold | | fold | |
| | | | induction | | induction | | induction | |
| Treatment | Conc | | Mean | SD | Mean | SD | Mean | SD |
| Vehicle | 0.1%(v/v) | | 100 | 0.1 | 1.0 | 0.1 | 1.0 | 0.2 |
| TGF-β1 0.1 | 0.1 | ng/mL | 1.0 | 0.2 | 0.9 | 0.1 | 1.2 | 0.2 |
| TGF-β1 10 | 10 | ng/mL | 0.8 | 0.1 | 1.7 | 0.1 | 1.1 | 0.2 |
| MTX 0.1 | 0.1 | μM | 1.2 | 0.1 | * | * | * | * |
| MTX 1 | 1 | μM | 1.1 | 0.1 | 0.8 | 0.1 | 0.7 | 0.1 |
| Vehicle | 0.1%(v/v) | | 0.8 | 0.1 | 1.0 | 0.1 | 1.1 | 0.5 |
| TGF-β1 0.1 | 0.1 | ng/mL | 0.8 | 0.0 | 1.0 | 0.0 | 1.2 | 0.1 |
| TGF-β1 10 | 10 | ng/mL | 0.8 | 0.1 | 1.6 | 0.2 | 0.1 | 0.1 |
| MTX 0.1 | 0.1 | μM | 0.9 | 0.1 | 1.0 | 0.1 | 1.0 | 0.1 |
| MTX 1 | 1 | μM | 1.1 | 0.3 | 1.1 | 0.2 | 0.6 | 0.2 |

* Results that cannot be calculated are for treatment groups that could not be r0.1un with all Taqman assays due to low mRNA levels

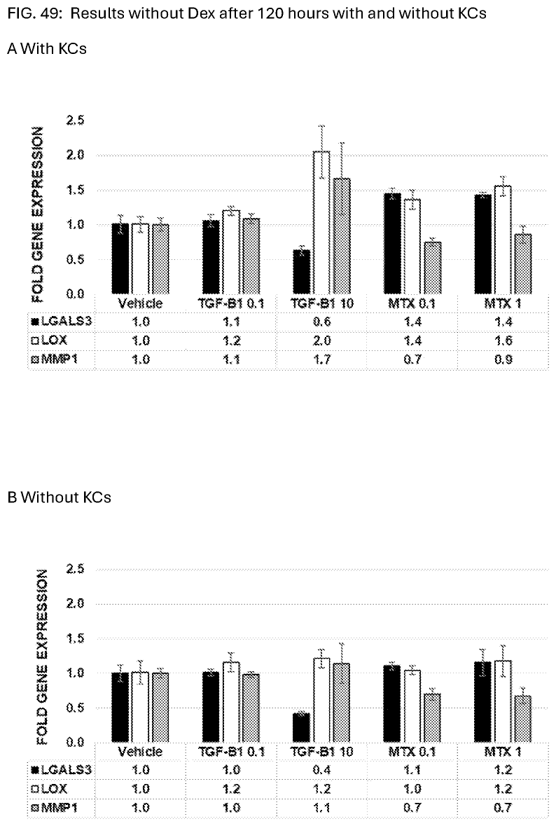

TABLE 6
(FIG. 49): Results after 120 hours without Dex with and without KCs
No KC

| | | | LGALS3 | | LOX | | MMP1 | |
|---|---|---|---|---|---|---|---|---|
| | | | fold | | fold | | fold | |
| | | | induction | | induction | | induction | |
| Treatment | Conc | | Mean | SD | Mean | SD | Mean | SD |
| Vehicle | 0.1%(v/v) | | 1.0 | 0.1 | 1.0 | 0.2 | 1.0 | 0.1 |
| TGF-β1 0.1 | 0.1 | ng/mL | 1.0 | 0.1 | 1.2 | 0.1 | 1.0 | 0.0 |
| TGF-β1 10 | 10 | ng/mL | 0.4 | 0.0 | 2.2 | 0.1 | 1.1 | 0.3 |
| MTX 0.1 | 0.1 | μM | 1.1 | 0.1 | 1.0 | 0.1 | 0.7 | 0.1 |
| MTX 1 | 1 | μM | 0.2 | 0.2 | 1.2 | 0.2 | 0.7 | 0.1 |
| Vehicle | 0.1%(v/v) | | 1.0 | 0.1 | 1.0 | 0.1 | 1.0 | 0.1 |
| TGF-β1 0.1 | 0.1 | ng/mL | 1.1 | 0.1 | 1.2 | 0.1 | 1.1 | 0.1 |
| TGF-β1 10 | 10 | ng/mL | 0.6 | 0.1 | 2.0 | 0.4 | 1.7 | 0.5 |
| MTX 0.1 | 0.1 | μM | 1.4 | 0.1 | 1.4 | 0.1 | 0.7 | 0.1 |
| MTX 1 | 1 | μM | 1.4 | 0.0 | 1.5 | 0.1 | 0.9 | 0.1 |

Results: All hepatic tri-cultures (both with and without the KCs) remained healthy over the initial culture period. KC presence was confirmed with CD-68 staining. Following compound (MX or TGF-β1) exposure (up to 120 hr), only those cultures exposed to 10 ng/ml TGF-β1 exhibited signs of cell stress with changes in hepatocellular morphology, urea production, and LDH release. The strongest gene responses following MTX and TFG-β1 exposure occurred with glucocorticoid-free media. In addition, compound, concentration, and time-dependent fibrogenic response gene changes were observed. MTX, at both concentrations, in the presence of KCs, increased LGALS3 gene expression at 72 hr (≥1.5-fold) with a slight decline at 120 hr. LOX increased at 72 hr and remained so at 120 hr (≥1.5-fold) with 1 μM MTX, while MMP1 slightly increased (1.4-fold) at 72 hr only. In contrast, TGF-β1, at 10 ng/ml, increased MMP1 in a time-dependent manner in the presence of KCs. Other genes (e.g., MMP14, PPARγ) responded in the opposite manner, with increased expression at earlier time points followed by a decline over exposure time. TGF-β1, at 0.1 ng/ml, did not affect gene expression. The tri-cultured hepatic system with KCs in a modified medium provides an in vitro platform that can be used to screen for potential compound-induced fibrogenic responses.

TGF β effects were observed in both DEX and no DEX treated groups, with or without KCs. This would be expected by a direct-acting activator of fibrogenic responses.

Methotrexate effects were observed on the no DEX treated groups with KCs. Without wishing to be constrained by theory, this could indicate that the full complement of cells was needed to follow the progression of hepatocellular injury followed by activation of immune cells, release of bioactivating cytokines, activation of stellate cells and increases in fibrogenic target genes.

The exposure conditions and time course (pre-treatment period; initiation, longevity and frequency of treatment) may be as follows.
KC: hepatocyte ratios of 1:10-1:2
Maximize cytokine profiles (IL6, TNF-α, TGF-β)

Time of treatment to time of harvest could be from 8 hours to 7 days. The collagen type 1 and other endpoints along sequence of events in vivo such as collagen 1 production and deposition may be monitored.

The timing of the KC addition to the plated hepatocytes may be changed. The time at which the KCs are added may have an effect on the results. For example the KCs may be added at 1, 2, 3, 4, 5, 6, 7, 8, 9, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours post-plating. The KCs may be added at any time post-plating. For example, the KCs may be added at 72, 84, 96,108,120,132, 144, 156, or 168 hours, 10 days, or 2 weeks post-plating, for example. The addition of the KC or other competent immune cells (macrophages) has been shown to be important to establishing an effective model.

The use of appropriate medium formulation may have effect on outcomes for example, the presence/absence of glucocorticoids may have an effect on producing a significant fibrogenic response). Thawing and/or plating the hepatocytes with the glucocorticoids may or may not have an effect of the response of the cells to the compounds. The base medium may play a role in optimal signal to noise ratio (e.g., DMEM vs WEM based media). After hepatocyte attachment, addition of glucocorticoid free culture medium was used except for the arm that had the 1 uM Dex present throughout the study. After addition of KCs on second day of culture, switching to half volume of glucocorticoid free medium components may have an effect on results.

Treatment with modulators/activators of fibrogenic responses. A combination of time periods between no treatment and treatment with modulators may be important to observed effects. Cultivation of hepatocytes together with KCs for a period to allow recovery/quiescence of cells. For example, this may be for 1, 2, 3, 4, 5, 6, 7, days, or may be for 4 days. The KCs may be added after different time periods. The cells may be treated with active agents for a sufficient period of time to observe changes in fibrogenic marker gene expression by direct vs indirect acting modulators. The amount of time may be from 8 hours to 5 days depending on the marker gene. Agent and/or the cytokine may affect appropriate exposure times. The agents may be direct acting, or they may be indirect acting. The frequency of medium changes, such as daily changes or changing the medium only once may have an effect on the results. The results may also be affected by adding the agent daily or adding it only once during the course of treatment.

### Example 10

This example is intended to elucidate the gene expressions (LGALS3 (Galectin 3), LOX (LysylOxidase), MMP1 (Matrix Metalloproteinase 1), and HKG=beta Actin) at earlier time points than the Example 9 results, for exposure of the cells to TGF-β.

### Design:

A 24-well plate, all-human cell-based triculture system (hTCS) that includes cryopreserved primary human feeder cells (FCs) and primary human hepatocytes (PHHs) and KC were used. The ratios of hepocytes: KC of 1:4 and 1:2 were used. The cells were exposed to TGF-β (10 ng/ml), with NO DEX in the media. During exposure, the above genes were followed. The workflow was the same as described in Example 9.

The cells were exposed to 10 ng/ml TGF-β.

Exposure times were measured at 2, 6, 24, and 96 hours. The gene expressions SMAD-7, LOX, MMP1, LGALS3, UCHL1, HSP90B1, MMP14, PPAR-λ(gamma)) and two housekeeping genes; β-Actin (duplexed) and GAPDH were measured. Cell Health was evaluated by measuring lactate dehydrogenase (LDH), and urea production at all time points.

IHC (Immunohistochemistry) was used on the 96 hr plate to measure CD68, Hepatocyte Nuclear Factor 4 (HNF4), and DAPI.

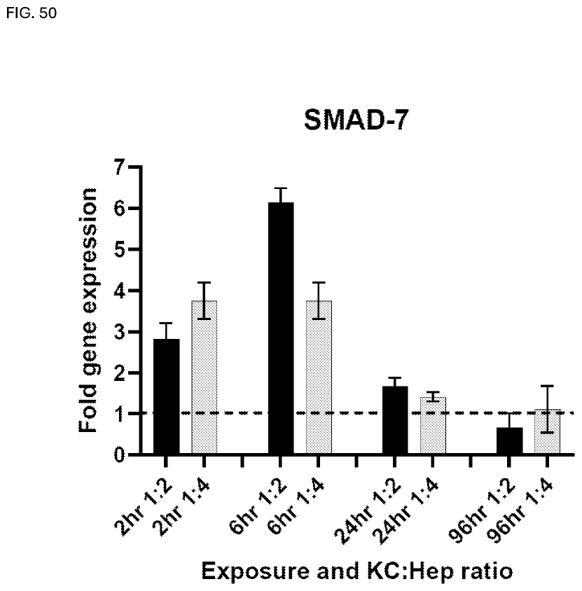

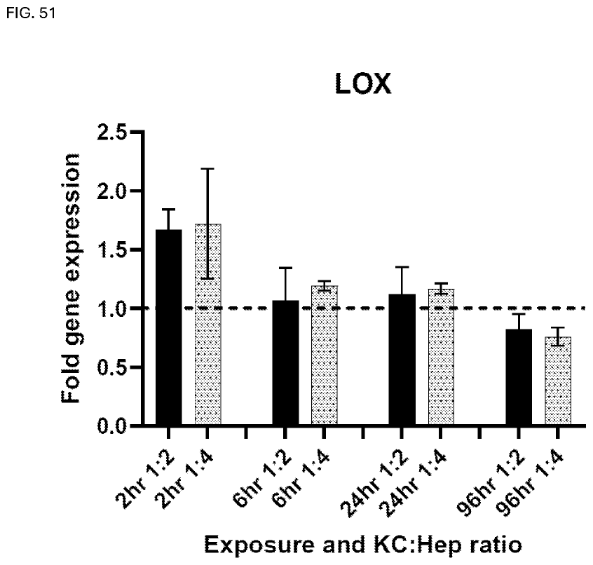

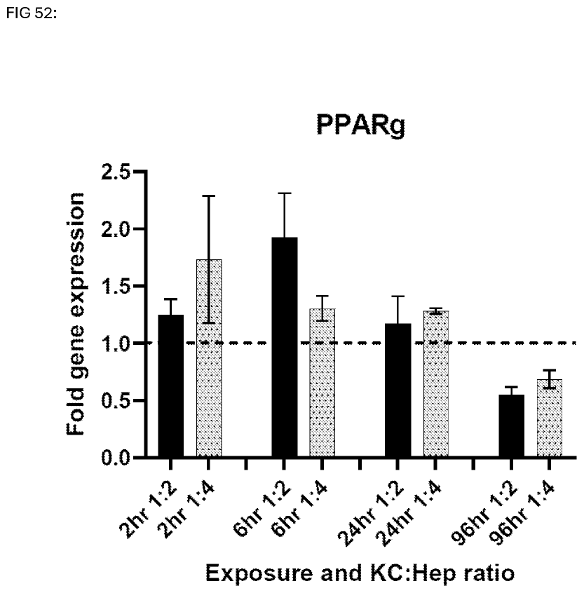

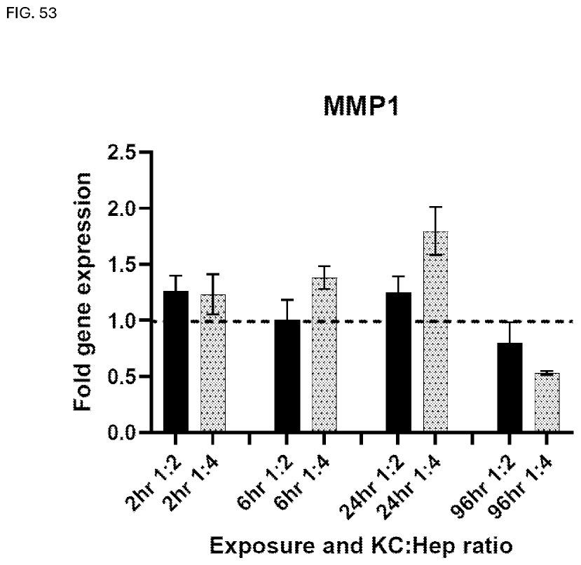

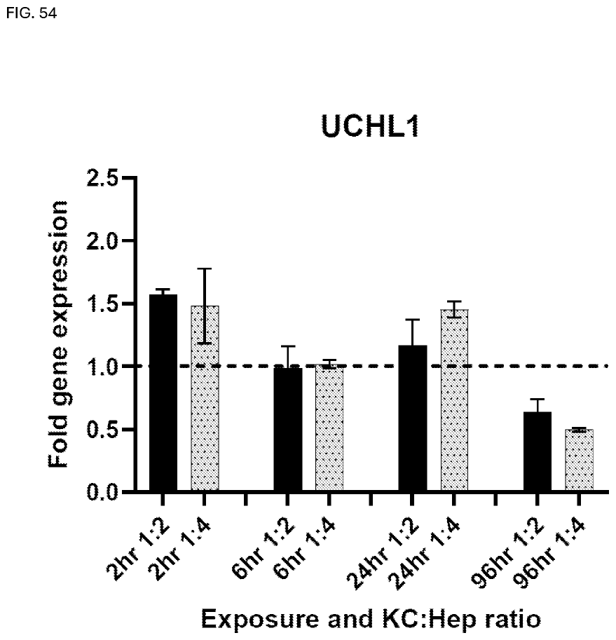

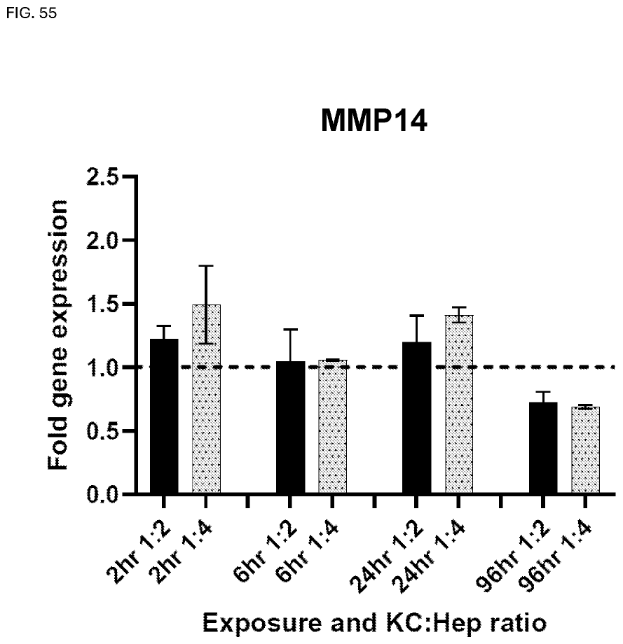

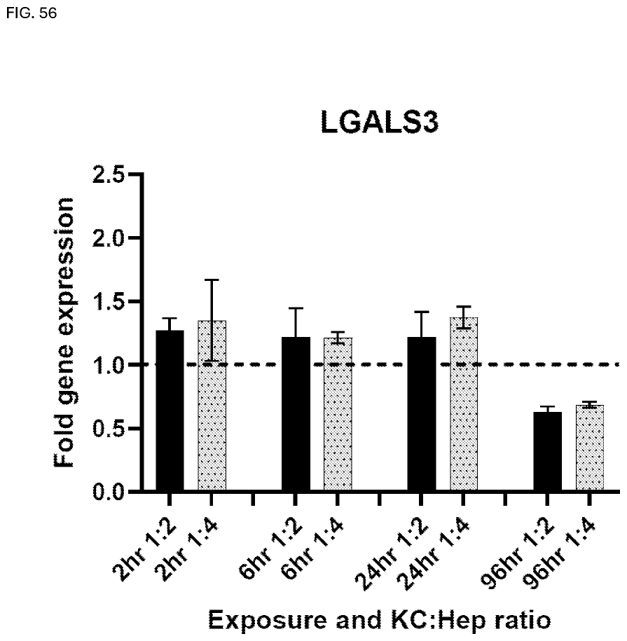

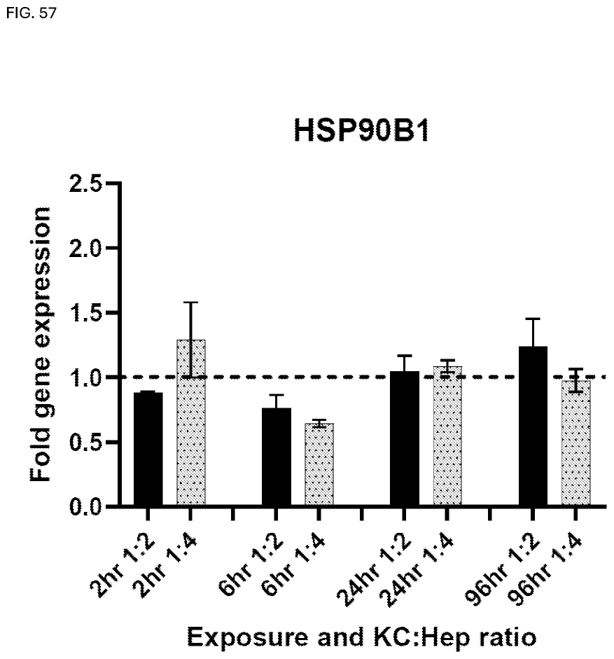

The results are summarized in FIGS. 50-57.

As shown in the figures, at 24 hours of exposure to TGF, multiple positive signals for gene expression were seen at both 1:2 and 1:4 ratios of hepatocytes to Kupffer cells. This batch of KCs was a different batch than was used for the results shown in the other time frames.

The ratio of the KCs to the hepatocytes affects the results. The results were consistent across the delivery platforms when the ratio of KC to hepatocytes was 1:4, compared to the 1:2 ratio. Without being bound to theory, it may be that negative feedback loops and the mechanisms of uptake may be important to the gene expressions.

The results also demonstrate that a prolonged exposure of 96 hours to TGF-β had a reduced gene expression compared to the controls for almost all of the genes at both ratios of 1:4 and 1:2 of Kupffer cells to hepatocytes.

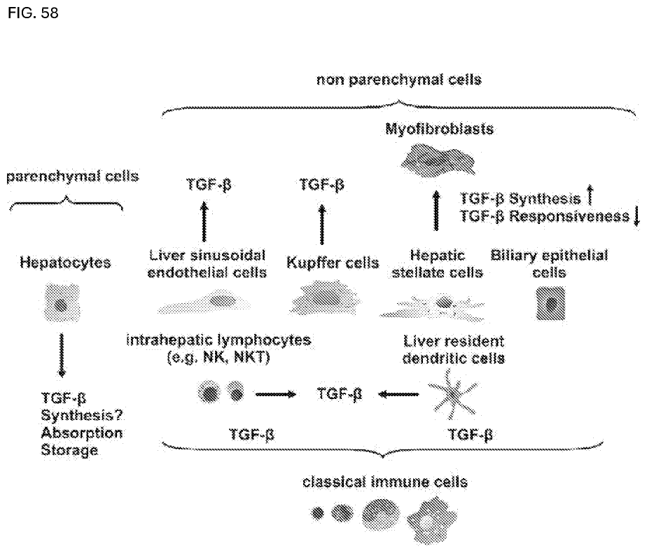

FIG. 58 shows in schematic form, that the TGF-β synthesis in the liver is done primarily in the non-parenchymal cells.

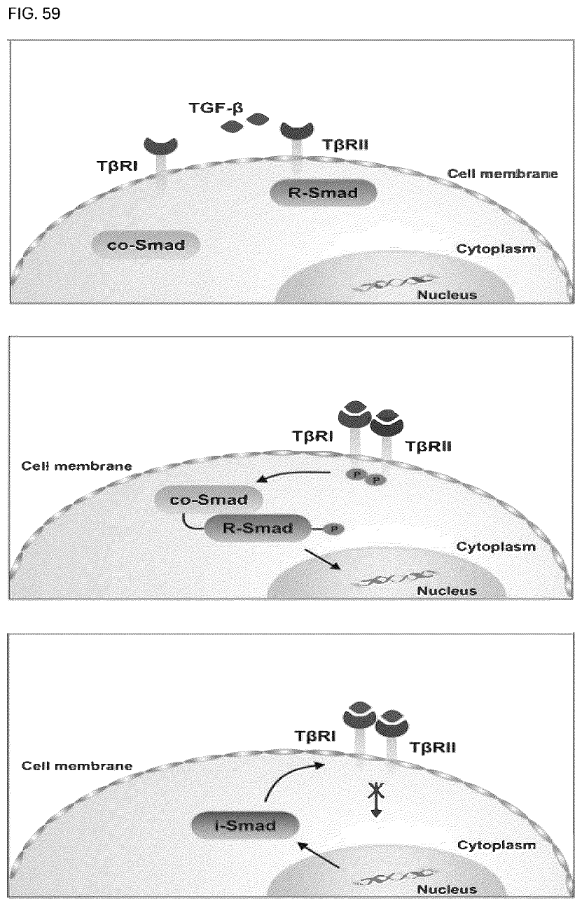

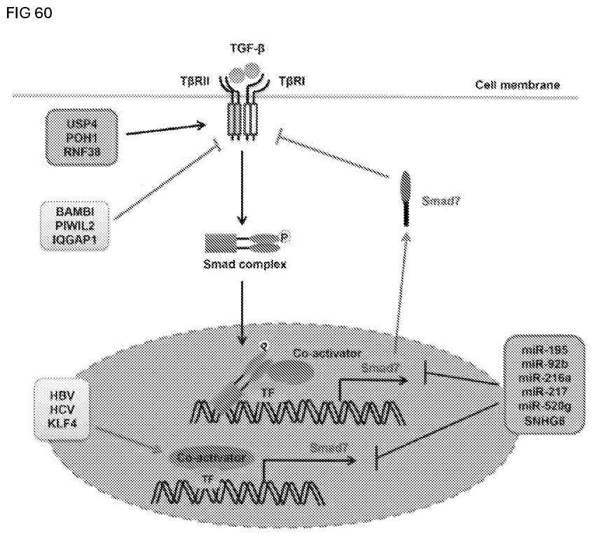

FIGS. 59 and 60 show together in schematic form how a negative feedback loop of the reduced TGF-β synthesis in the liver may be due to a negative feedback loop via the Smad 7 protein complex. This may be because the Smad 7 is an inhibitor of TGF-β signaling.

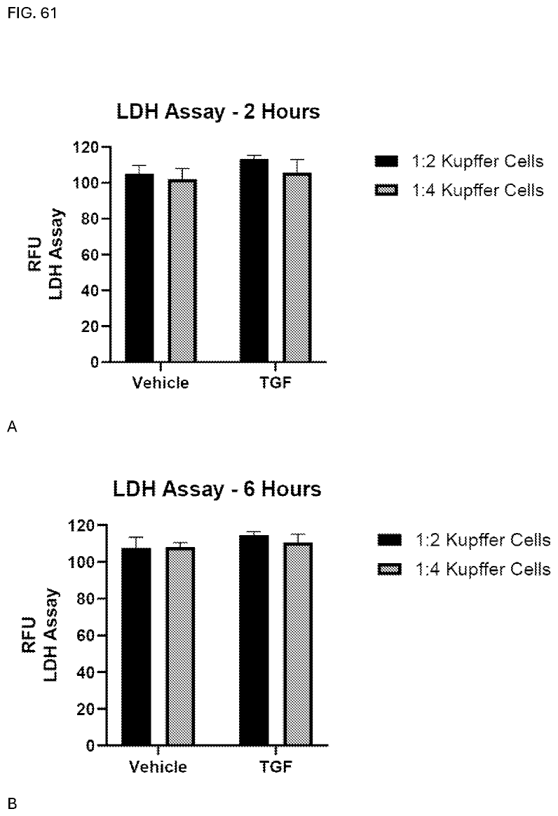

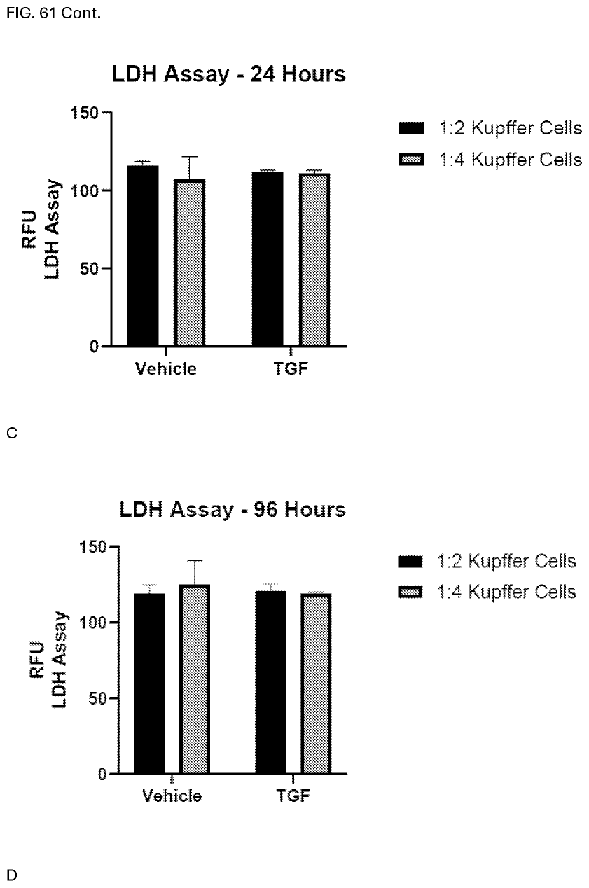

However, and importantly, FIGS. 61A, 61B, 61C, and 61D demonstrate that after 96 hours exposure the cell viability remained high, as expressed by the LDH (lactate dehydrogenase) leakage results. In some cases, the LDH leakage was even higher at 96 hours than at 2 hours, indicating that the cells remained viable and even improved in health over time.

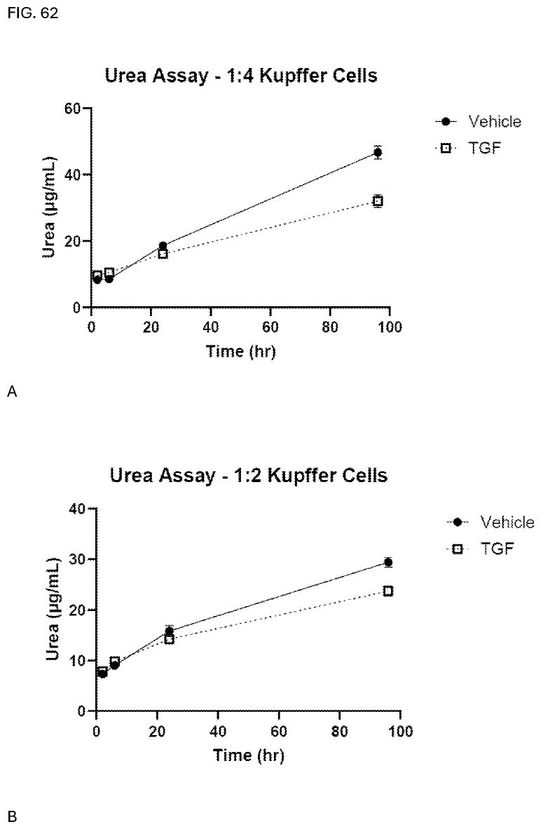

FIGS. 62A and 62B show cell health as a function of urea production over time for the two ratios of Kupffer cells to hepatocytes. Because the media was not changed, the urea production shown in these plots is cumulative and therefore is expected to increase over time as shown in these results. Urea production is linear between 6 and 96 h for all groups, although a decrease in the production rate is observed in the group treated with TGFbeta compared to those treated with vehicle alone, suggesting some impact on the synthetic capacity and health of the cells, which is also reflected in the microscope images of the cultures.

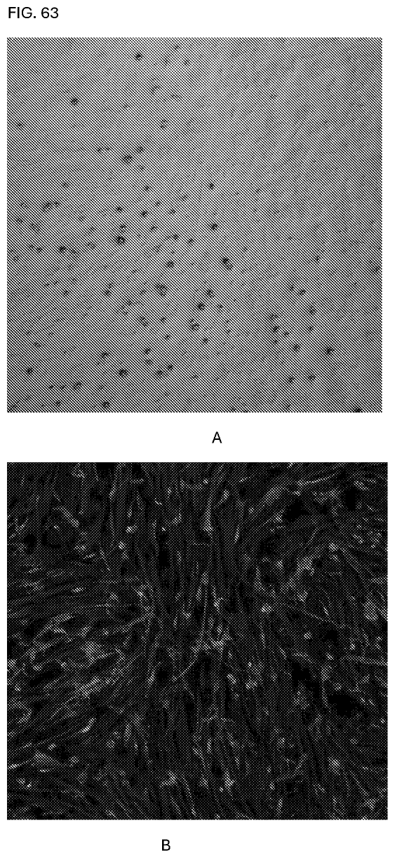

FIGS. 63A and 63B show (A) confocal brightfield and (B) IFC results for anti-CD68 and anti-HNF-4-alpha results for the same cell field after 96 hours of exposure for the 1:2 Kupffer cells to hepatocytes ratio. These show the presence of the CD68-positive cells (macrophages) with more anti-HNF-4-alpha (hepatocytes) than anti-CD68.

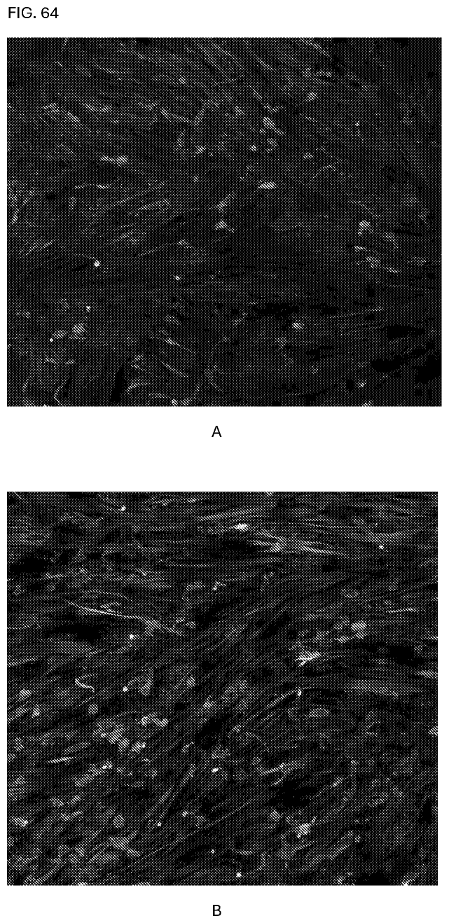

FIGS. 64A and 64B show a comparison of the IFC results for anti-CD68 and anti-HNF-4-alpha results after 96 hours of exposure for 1:2 and 1:4 Kupffer cells to hepatocytes ratios. These results provide qualitative verification of the presence of the Kupffer cells in the cultures at both titers.

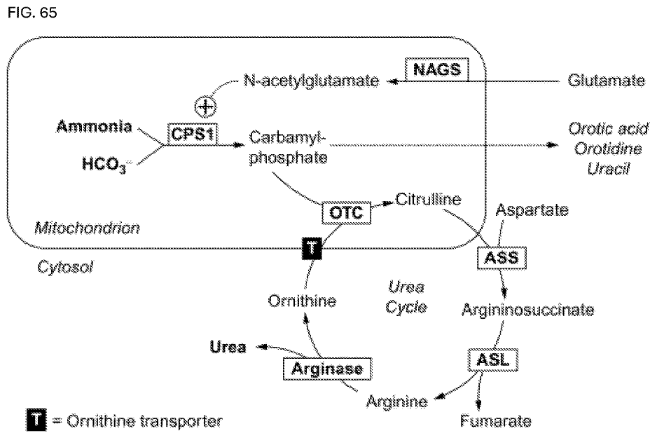

FIG. 65 shows in schematic form, how urea is synthesized in the liver. As shown in the figure, the urea cycle is partly cytoplasmic and partly mitochondrial. Only the liver possesses all the enzymes required to synthesize urea from ammonia, and this pathway is strictly located in periportal hepatocytes. Therefore, the urea production as shown in FIGS. 62A and 62B is only possible in the co-cultured liver cells, since non-parenchymal cells are needed to produce the urea.

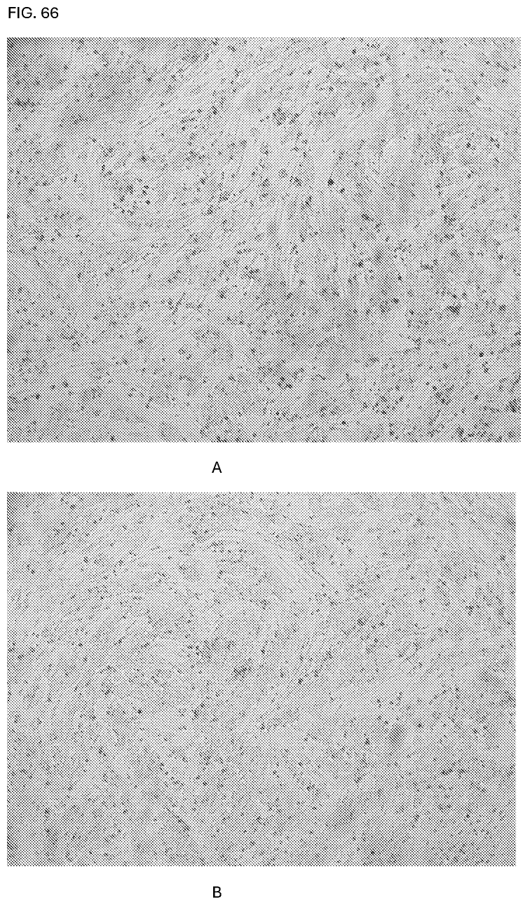

FIGS. 66A and 66B show at 10λ, the co-cultured cells at a 1:2 Kupffer cell to hepatocyte ratio at 24 hours of exposure without (A) the TFG-β and with (B) the TFG-β. As can be seen in FIGs, the cells appear healthy in both situations.

The Example 10 results demonstrate that the assays as performed successfully captured changes in the target genes due to exposure to the TFG-β. The results also show that exposure time is important for detecting positive effects.

The concentration of the Kupffer cells may have a more significant effect on TGF-β-induced changes in fibrogenic gene expression at shorter exposure times. The co-cultured cells in the presence of the Kupffer cells looked healthy and the urea production was consistent, the urea production after 96 hours of exposure may indicate cell stress. However, based on the LDH production in the presence of the Kupffer cells, cytotoxicity of the co-cultured cells was low and the cells were healthy even after 96 hours of exposure.

### Example 11

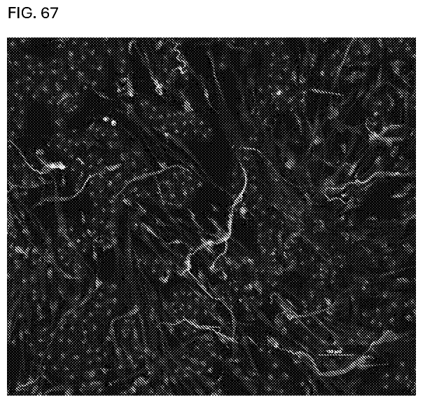

Hepatic tri-cultures with KCs at a 1:2 ratio were cultured in TCCM and Dex for 96 hr. IHC was performed as in the methods described above in Example 10 for CD68, HNF-4alpha, and DAPI. The immunostaining results are shown in FIG. 67. CD68-positive immune cells were stained green, HNF-4alpha-positive hepatocyte cells were stained red and the DAPI-stained nuclei of all cells were stained blue in FIG. 67. FIG. 67 demonstrates that that the CD68-positive immune cells (KCs) are attached and integrated into the TruVivo the culture system. The elongated morphology suggests that they are activated and migratory within and throughout the hepatocytes and feeder cells.

### Example 12

TruVivo® Culture Medium (TCCM) was obtained from LifeNet Health (Cat #MED-TCCM, Lot #22052003) and was used for culturing the cells.

### Medium Preparation

TCPM was prepared by adding 11 mL Supplement A (LifeNet Health, Cat #MED-TCSA, Lot #22004005) and 4.5 mL Supplement B (LifeNet Health, Cat #MED-TCSB, Lot #22046002) to the bottle of TCPM. TCCM was prepared by adding 22 mL Modified Supplement A (LifeNet Health, Lot #KKW08052023-09) and 1.5 mL Modified Supplement C (LifeNet Health, Cat #354352, Lot #1179002) to 100 mL TCCM. FC™ was transferred to a sterile 15 ml conical tube and mixed thoroughly. Human HH™ was mixed, filtered through a 0.2 μm filter, then transferred to a sterile 50 ml conical tube and mixed. Prior to use, each medium was warmed in a 37 C water bath for 20 to 30 minutes.

### Cell Plating and Culture

Human feeder cells were thawed, resuspended in FC™, and plated at a density target of 10,000 cells per well in TCPM on a collagen-coated 96-well plate according to the manufacturer's protocol. The culture plate was incubated at 37° C. with 5% CO₂ for 60 minutes. Once the feeder cells were successfully attached to the plate, primary human hepatocytes were thawed and resuspended in HH™. The culture plate with the feeder cells was removed from the incubator immediately before use. The media was aspirated from each well without disturbing the layer of feeder cells, and the hepatocytes were plated at a density of 30,000 cells per well in TCPM per manufacturer protocol. The plate was returned to the incubator. Hepatocyte attachment was inspected 2-4 hours post-plating, and the media in each well was replaced with 100 μL TCCM. The following day the human Kupffer cells were thawed and resuspended in TCCM. The media was aspirated from the wells the Kupffer cells were being added to, and the cells were plated at densities of 7,500 (1:4 ratio) cells per well in TCCM per manufacturer protocol. The culture plate was returned to the incubator. The culture was inspected daily, and the media was changed every day until dosing.

### Protein Secretion Assays

MIP1-α was measured using the Human MIP1-α CatchPoint ELISA kit from Abcam (Cat #ab229400-1001, Lot #2101046817, 2101048216). Reagents and standards were prepared according to the manufacturer's instructions. 50 μL of sample or standard and 50 μL of Antibody Cocktail were added to each well of a 96-well plate. The plate was sealed and incubated for one hour at room temperature, shaking at 400 rpm. Each well was washed with Wash Buffer, then 100 μL of CatchPoint HRP Development Solution was added to each well. The plate was incubated for 10 minutes in the dark, shaking at 400 rpm. 100 μL of Stop Solution was added to each well, and the plate was shaken for one minute. Fluorescence was read at 530 nm excitation and 590 nm emission using a BioTek Synergy H1 Plate Reader.

E-selectin secretion was measured using the CD62E Selectin Human ELISA kit from Abcam (Cat #ab100512, Lot #1067449-1, 1074590-1). Reagents and standards were prepared according to the manufacturer's instructions. 100 μL of sample or standard were added to the appropriate wells of a 96-well plate. The wells were covered, and the plate was incubated for 2.5 hours at room temperature gently shaking. The solution was discarded, the wells were washed with Wash Buffer, and 100 μL CD62E Detection Antibody was added to each well. The plate was incubated for 1 hour at room temperature with gentle shaking. The solution was discarded, the wells were washed, and 100 μL HRP-Streptavidin solution was added to each well. The plate was incubated for 45 minutes at room temperature gently shaking, the solution was discarded, and the wash process was repeated. 100 μL of TMB Substrate Reagent was added to each well. The plate was incubated for 30 minutes in the dark at room temperature gently shaking. 50 μL of Stop Solution was added to each well, and the OD was read at 450 nm using a BioTek Synergy H1 Plate Reader.

Interleukin 8 is a chemokine produced by macrophages and other cell types such as epithelial cells, airway smooth muscle cells and endothelial cells.

Secretion of IL-8 was measured using IL-8 (Cat #ab214030, Lot #2101039811) ELISA lit. Reagents and standards were prepared according to the manufacturer's instructions. 50 μL of sample or standard and 50 μL of Antibody Cocktail were added to each well of a 96-well plate. The plate was sealed and incubated for one hour at room temperature, shaking at 400 rpm. Each well was washed with Wash Buffer, then 100 μl of TMB Development Solution was added to each well. The plate was incubated for 10 minutes in the dark, shaking at 400 rpm. 100 μL of Stop Solution was added to each well, and the plate was shaken for one minute. Optical Density (OD) was read at 450 nm using a BioTek Synergy H1 Plate Reader.

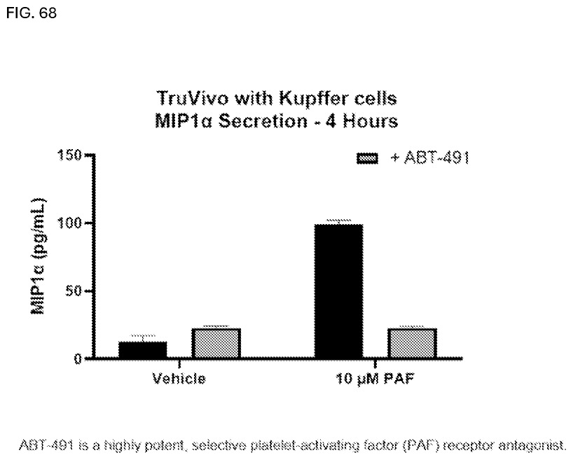

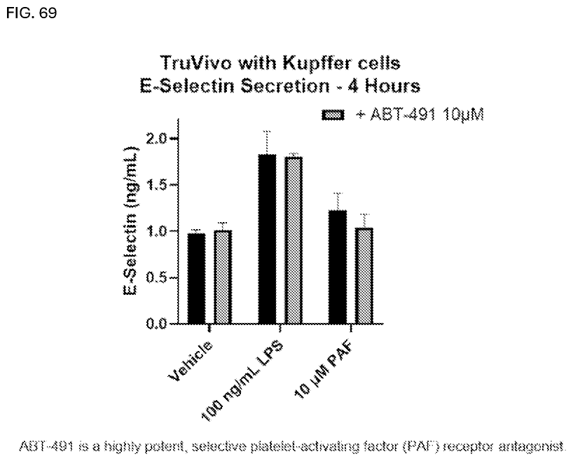

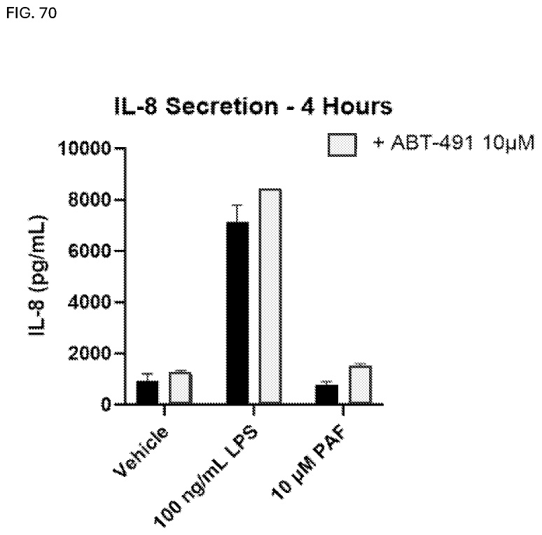

The results are shown in FIGS. 68-70. In FIGS. 68-70 the bar on the left in each pair is the treatment alone and the bar on the right in each pair is the treatment plus the added agent.

Examples 9-13 demonstrate that human hepatic tri-cultured system with KCs model system successfully captured effects on target genes with fibrogenic compounds. Both the presence of KCs and the absence of DEX in the medium had a positive impact on target gene expression. Transient expression of fibrogenic response genes was observed, possibly through a Smad-mediated feedback loop, indicating that exposure time is important for detecting early vs progressive effects. The results may indicate that KC titers may be more important at shorter exposure times. Cytotoxicity based on LDH was low, and hepatocytes and feeder cells appeared healthy; urea production was consistent after extended exposure to TGFβ, but a reduced production may indicate some stress at 96 hr. Overall, the results suggest that the human hepatic tri-cultured system with KCs system demonstrates a successful tool to assess immune-mediated toxicities.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A product comprising plated hepatocytes on a surface, wherein the plated hepatocytes are neonatal or juvenile hepatocytes, at least 70% of the plated hepatocytes are in one or more hepatocyte clusters on feeder cells, the feeder cells are endothelial cells and stromal cells and are attached to the surface, and the plated hepatocytes and the feeder cells are obtained from one or more donors.

2. A product comprising plated hepatocytes on a surface and Kupffer cells, at least 70% of the plated hepatocytes are in one or more hepatocyte clusters on feeder cells, the feeder cells are endothelial cells and stromal cells and are attached to the surface, and the plated hepatocytes and the feeder cells are obtained from one or more donors.

3. The product of claim 2, wherein the ratio of the Kupffer cells to the plated hepatocytes is between about 1:100 and about 4:1, or from about 1:100 to about 2:1 or from about 1:100 to about 1:1, or from about 1:100 to about 1:2.

4. The product of claim 2, wherein the Kupffer cells are from a healthy donor.

5. The product of claim 2, wherein the Kupffer cells are from a donor who has suffered from a liver disease or condition selected from the group consisting of microsteatosis, metabolic dysfunction-associated steatohepatitis (MASH), Metabolic dysfunction-Associated Fatty Liver Disease (MAFLD) and hepatitis.

6. The product of claim 2, wherein the Kupffer cells are from a donor who shows a pathology selected from the group consisting of metabolic dysfunction-associated steatohepatitis (MASH), Metabolic dysfunction-Associated Fatty Liver Disease (MAFLD), alcoholic steatohepatitis (ASH), alpha-1-antitrypsin deficiency, Wilson's disease, Hepatitis B, Hepatitis C, Hemochromatosis, hyperoxaluria, autoimmune disease, cancer, obesity, type 2 diabetes, or an inborn error of metabolism.

7. The product of claim 1, wherein the plated hepatocytes are neonatal hepatocytes.

8. The product of claim 1, wherein the plated hepatocytes are juvenile hepatocytes.

9. The product of claim 2, wherein the plated hepatocytes are adult hepatocytes.

10. The product of claim 1, wherein the plated hepatocytes are obtained from a single donor.

11. The product of claim 1, wherein the plated hepatocytes are obtained from two or more donors.

12. A method of testing a pharmaceutical substance, comprising administering a pharmaceutical substance to the plated hepatocytes in the product of claim 1 in an amount effective to change a property of the plated hepatocytes.

13. The method of claim 12, wherein the pharmaceutical substance is selected from the group consisting of small molecules, lipid nanoparticles, bacteria or derivatives thereof, antibodies, live viruses, viral vectors, oligonucleotides and cells.

14. A method of testing drug metabolism, comprising administering an effective amount of a drug to the plated hepatocytes in the product of claim 1, and determining the amount of the drug in the plated hepatocytes.

15. A method of testing drug transport, comprising administering an effective amount of a drug to the plated hepatocytes in the product of claim 1, and determining the location of the drug in the plated hepatocytes.

16. A method of testing drug toxicity, comprising administering an effective amount of a drug to the plated hepatocytes in the product of claim 1, and detecting viable plated hepatocytes.

17. A kit for plating hepatocytes, comprising
(a) a first cryovial comprising primary hepatocytes, wherein the primary hepatocytes are neonatal or juvenile hepatocytes;
(b) a second cryovial comprising endothelial cells;
(c) a third cryovial comprising stromal cells; and
(d) an instruction for preparing plated hepatocytes with the primary hepatocytes, the endothelial cells and the stromal cells according to a method comprising:
(i) applying the primary hepatocytes to a surface in the presence of feeder cells, wherein the feeder cells are the endothelial cells and the stromal cells,
(ii) co-culturing the applied hepatocytes with the feeder cells after step (i), and
(iii) forming one or more hepatocyte clusters by the co-cultured hepatocytes on the feeder cells, wherein the feeder cells are attached to the surface, wherein at least 85% of the co-cultured hepatocytes are in the one or more hepatocyte clusters.

18. A kit for plating hepatocytes, comprising
(a) a first cryovial comprising primary hepatocytes;
(b) a second cryovial comprising endothelial cells;
(c) a third cryovial comprising stromal cells;
(d) a fourth cryovial comprising Kupffer cells; and
(e) an instruction for preparing plated hepatocytes with the primary hepatocytes, the endothelial cells and the stromal cells according to a method comprising:
(i) applying the primary hepatocytes to a surface in the presence of feeder cells, wherein the feeder cells are the endothelial cells and the stromal cells,
(ii) co-culturing the applied hepatocytes with the feeder cells after step (i), and
(iii) forming one or more hepatocyte clusters by the co-cultured hepatocytes on the feeder cells, wherein the feeder cells are attached to the surface, wherein at least 85% of the co-cultured hepatocytes are in the one or more hepatocyte clusters.

19. The kit of claim 18, wherein the ratio of the Kupffer cells to the plated hepatocytes is between about 1:100 and about 4:1, or from about 1:100 to about 2:1 or from about 1:100 to about 1:1, or from about 1:100 to about 1:2.

20. The kit of claim 18, wherein the Kupffer cells are from a healthy donor.

21. The kit of claim 18, wherein the Kupffer cells are from a donor who has suffered from a liver disease or condition selected from the group consisting of microsteatosis, metabolic dysfunction-associated steatohepatitis (MASH), Metabolic dysfunction-Associated Fatty Liver Disease (MAFLD) and hepatitis.

22. The kit of claim 18, wherein the Kupffer cells are from a donor who shows a pathology selected from the group consisting of metabolic dysfunction-associated steatohepatitis (MASH), Metabolic dysfunction-Associated Fatty Liver Disease (MAFLD), alcoholic steatohepatitis (ASH), alpha-1-antitrypsin deficiency, Wilson's disease, Hepatitis B, Hepatitis C, Hemochromatosis, hyperoxaluria, autoimmune disease, cancer, obesity, type 2 diabetes, or an inborn error of metabolism.

23. The kit of claim 17, wherein the plated hepatocytes are neonatal hepatocytes.

24. The kit of claim 17, wherein the plated hepatocytes are juvenile hepatocytes.

25. The kit of claim 17, wherein the second cryovial and the third cryovial are the same.

26. The product of claim 1, wherein the stromal cells are fibroblasts.

27. The kit of claim 17, wherein the stromal cells are fibroblasts.

28. The kit of claim 17, wherein the second vial and the third vial are combined into a single vial.
